SEQUENCE LISTING (104 sequences)

SEQ ID NO: 1 - protein, 222 aa, synthetic construct
  REGION 1..222: note = Membrane Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 2 - DNA, 669 bp, synthetic construct
  misc_feature 1..669: note = Membrane Protein
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 3 - protein, 75 aa, synthetic construct
  REGION 1..75: note = Envelope Protein
MYSFVSEETGTLIVNSVLLFLAFVVFLLVTLAILTALRLCAYCCNIVNVSLVKPSFYVYSRVKNLNSSRVPDLLV

SEQ ID NO: 4 - DNA, 228 bp, synthetic construct
  misc_feature 1..228: note = Envelope Protein
  source 1..228: mol_type = other DNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 5 - protein, 1273 aa, synthetic construct
  REGION 1..1273: note = Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - DNA, 1118 bp, synthetic construct
  misc_feature 1..1118: note = Spike protein
  source 1..1118: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatc

SEQ ID NO: 7 - protein, 18 aa, synthetic construct
  REGION 1..18: note = Internal Fusion Peptide
SFIEDLLFNKVTLADAGF

SEQ ID NO: 8 - DNA, 54 bp, synthetic construct
  misc_feature 1..54: note = Internal Fusion Peptide
  source 1..54: mol_type = other DNA
tcatttattgaagatctacttttcaacaaagtgacacttgcagatgctggcttc

SEQ ID NO: 9 - protein, 254 aa, synthetic construct
  REGION 1..254: note = receptor-binding domain
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 10 - DNA, 762 bp, synthetic construct
  misc_feature 1..762: note = receptor-binding domain
  source 1..762: mol_type = other DNA
cctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgag

SEQ ID NO: 11 - protein, 192 aa, synthetic construct
  REGION 1..192: note = immunogenic sequence
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 12 - protein, 6 aa, synthetic construct
  REGION 1..6: note = Conserved Amino Acid Sequence
SFIEDL

SEQ ID NO: 13 - protein, 5 aa, synthetic construct
  REGION 1..5: note = Conserved Amino Acid Sequence
GVYYP

SEQ ID NO: 14 - protein, 4 aa, synthetic construct
  REGION 1..4: note = Conserved Amino Acid Sequence
FLPF

SEQ ID NO: 15 - protein, 4 aa, synthetic construct
  REGION 1..4: note = Conserved Amino Acid Sequence
VLPF

SEQ ID NO: 16 - protein, 4 aa, synthetic construct
  REGION 1..4: note = Conserved Amino Acid Sequence
SLLI

SEQ ID NO: 17 - protein, 5 aa, synthetic construct
  REGION 1..5: note = Conserved Amino Acid Sequence
LPIGI

SEQ ID NO: 18 - protein, 5 aa, synthetic construct
  REGION 1..5: note = Conserved Amino Acid Sequence
AAYYV

SEQ ID NO: 19 - protein, 4 aa, synthetic construct
  REGION 1..4: note = Conserved Amino Acid Sequence
TFLL

SEQ ID NO: 20 - protein, 4 aa, synthetic construct
  REGION 1..4: note = Conserved Amino Acid Sequence
AVDC

SEQ ID NO: 21 - protein, 5 aa, synthetic construct
  REGION 1..5: note = Conserved Amino Acid Sequence
IVRFP

SEQ ID NO: 22 - protein, 4 aa, synthetic construct
  REGION 1..4: note = Conserved Amino Acid Sequence
ISNC

SEQ ID NO: 23 - protein, 4 aa, synthetic construct
  REGION 1..4: note = Conserved Amino Acid Sequence
LCFT

SEQ ID NO: 24 - protein, 5 aa, synthetic construct
  REGION 1..5: note = Conserved Amino Acid Sequence
YNYKL

SEQ ID NO: 25 - protein, 4 aa, synthetic construct
  REGION 1..4: note = Conserved Amino Acid Sequence
IAWN

SEQ ID NO: 26 - protein, 6 aa, synthetic construct
  REGION 1..6: note = Conserved Amino Acid Sequence
VVVLSF

SEQ ID NO: 27 - protein, 4 aa, synthetic construct
  REGION 1..4: note = Conserved Amino Acid Sequence
CVNF

SEQ ID NO: 28 - protein, 4 aa, synthetic construct
  REGION 1..4: note = Conserved Amino Acid Sequence
GLTG

SEQ ID NO: 29 - protein, 5 aa, synthetic construct
  REGION 1..5: note = Conserved Amino Acid Sequence
VAVLY

SEQ ID NO: 30 - protein, 4 aa, synthetic construct
  REGION 1..4: note = Conserved Amino Acid Sequence
GCLI

SEQ ID NO: 31 - protein, 4 aa, synthetic construct
  REGION 1..4: note = Conserved Amino Acid Sequence
GICA

SEQ ID NO: 32 - protein, 4 aa, synthetic construct
  REGION 1..4: note = Conserved Amino Acid Sequence
FTIS

SEQ ID NO: 33 - protein, 4 aa, synthetic construct
  REGION 1..4: note = Conserved Amino Acid Sequence
SVDC

SEQ ID NO: 34 - protein, 5 aa, synthetic construct
  REGION 1..5: note = Conserved Amino Acid Sequence
YGSFC

SEQ ID NO: 35 - protein, 4 aa, synthetic construct
  REGION 1..4: note = Conserved Amino Acid Sequence
FNFS

SEQ ID NO: 36 - protein, 7 aa, synthetic construct
  REGION 1..7: note = Conserved Amino Acid Sequence
RDLICAQ

SEQ ID NO: 37 - protein, 6 aa, synthetic construct
  REGION 1..6: note = Conserved Amino Acid Sequence
VLPPLL

SEQ ID NO: 38 - protein, 4 aa, synthetic construct
  REGION 1..4: note = Conserved Amino Acid Sequence
IPFA

SEQ ID NO: 39 - protein, 4 aa, synthetic construct
  REGION 1..4: note = Conserved Amino Acid Sequence
YRFN

SEQ ID NO: 40 - protein, 7 aa, synthetic construct
  REGION 1..7: note = Conserved Amino Acid Sequence
KLQDVVN

SEQ ID NO: 41 - protein, 5 aa, synthetic construct
  REGION 1..5: note = Conserved Amino Acid Sequence
GAISS

SEQ ID NO: 42 - protein, 8 aa, synthetic construct
  REGION 1..8: note = Conserved Amino Acid Sequence
EVQIDRLI

SEQ ID NO: 43 - protein, 6 aa, synthetic construct
  REGION 1..6: note = Conserved Amino Acid Sequence
YVTQQL

SEQ ID NO: 44 - protein, 5 aa, synthetic construct
  REGION 1..5: note = Conserved Amino Acid Sequence
HLMSF

SEQ ID NO: 45 - protein, 6 aa, synthetic construct
  REGION 1..6: note = Conserved Amino Acid Sequence
GVVHLF

SEQ ID NO: 46 - protein, 4 aa, synthetic construct
  REGION 1..4: note = Conserved Amino Acid Sequence
WFVT

SEQ ID NO: 47 - protein, 4 aa, synthetic construct
  REGION 1..4: note = Conserved Amino Acid Sequence
INAS

SEQ ID NO: 48 - protein, 4 aa, synthetic construct
  REGION 1..4: note = Conserved Amino Acid Sequence
LLQF

SEQ ID NO: 49 - protein, 6 aa, synthetic construct
  REGION 1..6: note = Conserved Amino Acid Sequence
LWLLWP

SEQ ID NO: 50 - protein, 4 aa, synthetic construct
  REGION 1..4: note = Conserved Amino Acid Sequence
LMWL

SEQ ID NO: 51 - protein, 5 aa, synthetic construct
  REGION 1..5: note = Conserved Amino Acid Sequence
SFRLF

SEQ ID NO: 52 - protein, 6 aa, synthetic construct
  REGION 1..6: note = Conserved Amino Acid Sequence
FNPETN

SEQ ID NO: 53 - protein, 4 aa, synthetic construct
  REGION 1..4: note = Conserved Amino Acid Sequence
ITVA

SEQ ID NO: 54 - protein, 4 aa, synthetic construct
  REGION 1..4: note = Conserved Amino Acid Sequence
LRLC

SEQ ID NO: 55 - protein, 245 aa, synthetic construct
  REGION 1..245: note = recombinant spike protein
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGGGGGGSFIEDLLFNKVTLADAGFGGGGGGWPWYIWLGFIAGLIAIVMVTIML

SEQ ID NO: 56 - DNA, 738 bp, synthetic construct
  misc_feature 1..738: note = recombinant spike protein
  source 1..738: mol_type = other DNA
ccaaacattaccaacctgtgccccttcggcgaggtgttcaacgccacacggttcgccagcgtgtacgcctggaacagaaagcggatcagcaactgcgtggccgactacagtgtcctgtataactccgccagcttttctacattcaagtgctacggcgtctcccctaccaagctgaacgacctgtgcttcaccaatgtgtacgccgattctttcgtgatcagaggcgacgaggtgcggcagatcgcccctggccagaccggaaagatcgctgattacaactacaagctgcctgatgacttcaccggctgcgtgatcgcctggaactccaacaacctggacagcaaggtggggggcaactacaactacctgtacagactgttcagaaagagcaatctgaagcctttcgagagagatatcagcacagagatctaccaggccggcagcaccccttgtaatggcgttgagggcttcaattgctactttccactgcagagctatggctttcagcctacaaacggcgtgggctaccaaccttacagagtggtggtgctgtctttcgagctgctgcacgcccctggcggaggaggaggcggatctttcatcgaggacctgctgttcaacaaggtgaccctggccgacgccggttttggcggtggcggcggcggctggccttggtacatctggctgggcttcatcgccggactgatcgccatcgtgatggtcaccatcatgctgtga

SEQ ID NO: 57 - protein, 580 aa, synthetic construct
  REGION 1..580: note = single open reading frame for coronavirus VLP
MYSFVSEETGTLIVNSVLLFLAFVVFLLVTLAILTALRLCAYCCNIVNVSLVKPSFYVYSRVKNLNSSRVPDLLVATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQATNFSLLKQAGDVEENPGPPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGGGGGGSFIEDLLFNKVTLADAGFGGGGGGWPWYIWLGFIAGLIAIVMVTIML

SEQ ID NO: 58 - DNA, 1743 bp, synthetic construct
  misc_feature 1..1743: note = single open reading frame for coronavirus VLP
  source 1..1743: mol_type = other DNA
atgtactctttcgtgtctgaggaaaccggcaccctgatcgtgaacagcgtgctgctgtttctggccttcgtggttttcctgctggtcaccctcgccatcctgaccgccctgcggctgtgcgcctactgctgcaacatcgtgaacgtgtctctggtcaaacctagcttctacgtgtatagccgggtgaagaacctgaattctagcagggtgcccgacctgctggtggccaccaacttcagcctgctgaaacaggctggcgatgtggaagagaaccctggacctatggccgatagcaacggcaccattacagtggaggaactcaaaaagctgctggaacagtggaatcttgtgatcggcttcctgttcctgacctggatctgcctgctgcagttcgcctacgccaaccgcaacagattcctgtacatcatcaaactgatcttcctgtggctgctgtggcccgtgaccctggcttgtttcgtgctggctgctgtttatagaatcaactggatcacaggcggcatcgcaatcgccatggcctgtctggtgggcctgatgtggctgagctacttcatcgccagctttagactgttcgctagaacaagaagcatgtggtcctttaaccccgagacaaacatcctcctgaatgtgccactgcatggcaccatcctgacaagacccctgctggaaagcgagctggtcatcggcgccgtgatcctgcggggccacctgagaatcgctggccaccacctgggcagatgtgacatcaaggacctgcccaaggaaatcactgtggccacaagcagaaccctcagctactacaagctgggagcctctcagagagtggccggcgacagcggcttcgccgcctacagccggtaccggattggcaattacaaactgaacaccgaccacagctccagcagcgacaacatcgctctgctagtgcaggccaccaatttcagcctgctgaagcaagctggagatgtggaagaaaaccccggccctccaaacattaccaacctgtgccccttcggcgaggtgttcaacgccacacggttcgccagcgtgtacgcctggaacagaaagcggatcagcaactgcgtggccgactacagtgtcctgtataactccgccagcttttctacattcaagtgctacggcgtctcccctaccaagctgaacgacctgtgcttcaccaatgtgtacgccgattctttcgtgatcagaggcgacgaggtgcggcagatcgcccctggccagaccggaaagatcgctgattacaactacaagctgcctgatgacttcaccggctgcgtgatcgcctggaactccaacaacctggacagcaaggtggggggcaactacaactacctgtacagactgttcagaaagagcaatctgaagcctttcgagagagatatcagcacagagatctaccaggccggcagcaccccttgtaatggcgttgagggcttcaattgctactttccactgcagagctatggctttcagcctacaaacggcgtgggctaccaaccttacagagtggtggtgctgtctttcgagctgctgcacgcccctggcggaggaggaggcggatctttcatcgaggacctgctgttcaacaaggtgaccctggccgacgccggttttggcggtggcggcggcggctggccttggtacatctggctgggcttcatcgccggactgatcgccatcgtgatggtcaccatcatgctgtga

SEQ ID NO: 59 - DNA, 2523 bp, synthetic construct
  misc_feature 1..2523: note = expression cassette for VLP
  source 1..2523: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccaccatgtactctttcgtgtctgaggaaaccggcaccctgatcgtgaacagcgtgctgctgtttctggccttcgtggttttcctgctggtcaccctcgccatcctgaccgccctgcggctgtgcgcctactgctgcaacatcgtgaacgtgtctctggtcaaacctagcttctacgtgtatagccgggtgaagaacctgaattctagcagggtgcccgacctgctggtggccaccaacttcagcctgctgaaacaggctggcgatgtggaagagaaccctggacctatggccgatagcaacggcaccattacagtggaggaactcaaaaagctgctggaacagtggaatcttgtgatcggcttcctgttcctgacctggatctgcctgctgcagttcgcctacgccaaccgcaacagattcctgtacatcatcaaactgatcttcctgtggctgctgtggcccgtgaccctggcttgtttcgtgctggctgctgtttatagaatcaactggatcacaggcggcatcgcaatcgccatggcctgtctggtgggcctgatgtggctgagctacttcatcgccagctttagactgttcgctagaacaagaagcatgtggtcctttaaccccgagacaaacatcctcctgaatgtgccactgcatggcaccatcctgacaagacccctgctggaaagcgagctggtcatcggcgccgtgatcctgcggggccacctgagaatcgctggccaccacctgggcagatgtgacatcaaggacctgcccaaggaaatcactgtggccacaagcagaaccctcagctactacaagctgggagcctctcagagagtggccggcgacagcggcttcgccgcctacagccggtaccggattggcaattacaaactgaacaccgaccacagctccagcagcgacaacatcgctctgctagtgcaggccaccaatttcagcctgctgaagcaagctggagatgtggaagaaaaccccggccctccaaacattaccaacctgtgccccttcggcgaggtgttcaacgccacacggttcgccagcgtgtacgcctggaacagaaagcggatcagcaactgcgtggccgactacagtgtcctgtataactccgccagcttttctacattcaagtgctacggcgtctcccctaccaagctgaacgacctgtgcttcaccaatgtgtacgccgattctttcgtgatcagaggcgacgaggtgcggcagatcgcccctggccagaccggaaagatcgctgattacaactacaagctgcctgatgacttcaccggctgcgtgatcgcctggaactccaacaacctggacagcaaggtggggggcaactacaactacctgtacagactgttcagaaagagcaatctgaagcctttcgagagagatatcagcacagagatctaccaggccggcagcaccccttgtaatggcgttgagggcttcaattgctactttccactgcagagctatggctttcagcctacaaacggcgtgggctaccaaccttacagagtggtggtgctgtctttcgagctgctgcacgcccctggcggaggaggaggcggatctttcatcgaggacctgctgttcaacaaggtgaccctggccgacgccggttttggcggtggcggcggcggctggccttggtacatctggctgggcttcatcgccggactgatcgccatcgtgatggtcaccatcatgctgtgaacggccggctgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 60 - DNA, 2510 bp, synthetic construct
  misc_feature 1..2510: note = expression cassette for VLP
  source 1..2510: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccaccatgtactctttcgtgtctgaggaaaccggcaccctgatcgtgaacagcgtgctgctgtttctggccttcgtggttttcctgctggtcaccctcgccatcctgaccgccctgcggctgtgcgcctactgctgcaacatcgtgaacgtgtctctggtcaaacctagcttctacgtgtatagccgggtgaagaacctgaattctagcagggtgcccgacctgctggtggccaccaacttcagcctgctgaaacaggctggcgatgtggaagagaaccctggacctatggccgatagcaacggcaccattacagtggaggaactcaaaaagctgctggaacagtggaatcttgtgatcggcttcctgttcctgacctggatctgcctgctgcagttcgcctacgccaaccgcaacagattcctgtacatcatcaaactgatcttcctgtggctgctgtggcccgtgaccctggcttgtttcgtgctggctgctgtttatagaatcaactggatcacaggcggcatcgcaatcgccatggcctgtctggtgggcctgatgtggctgagctacttcatcgccagctttagactgttcgctagaacaagaagcatgtggtcctttaaccccgagacaaacatcctcctgaatgtgccactgcatggcaccatcctgacaagacccctgctggaaagcgagctggtcatcggcgccgtgatcctgcggggccacctgagaatcgctggccaccacctgggcagatgtgacatcaaggacctgcccaaggaaatcactgtggccacaagcagaaccctcagctactacaagctgggagcctctcagagagtggccggcgacagcggcttcgccgcctacagccggtaccggattggcaattacaaactgaacaccgaccacagctccagcagcgacaacatcgctctgctagtgcaggccaccaatttcagcctgctgaagcaagctggagatgtggaagaaaaccccggccctccaaacattaccaacctgtgccccttcggcgaggtgttcaacgccacacggttcgccagcgtgtacgcctggaacagaaagcggatcagcaactgcgtggccgactacagtgtcctgtataactccgccagcttttctacattcaagtgctacggcgtctcccctaccaagctgaacgacctgtgcttcaccaatgtgtacgccgattctttcgtgatcagaggcgacgaggtgcggcagatcgcccctggccagaccggaaagatcgctgattacaactacaagctgcctgatgacttcaccggctgcgtgatcgcctggaactccaacaacctggacagcaaggtggggggcaactacaactacctgtacagactgttcagaaagagcaatctgaagcctttcgagagagatatcagcacagagatctaccaggccggcagcaccccttgtaatggcgttgagggcttcaattgctactttccactgcagagctatggctttcagcctacaaacggcgtgggctaccaaccttacagagtggtggtgctgtctttcgagctgctgcacgcccctggcggaggaggaggcggatctttcatcgaggacctgctgttcaacaaggtgaccctggccgacgccggttttggcggtggcggcggcggctggccttggtacatctggctgggcttcatcgccggactgatcgccatcgtgatggtcaccatcatgctgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 61 - DNA, 3273 bp, synthetic construct
  misc_feature 1..3273: note = expression cassette for VLP
  source 1..3273: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccaccatgtactctttcgtgtctgaggaaaccggcaccctgatcgtgaacagcgtgctgctgtttctggccttcgtggttttcctgctggtcaccctcgccatcctgaccgccctgcggctgtgcgcctactgctgcaacatcgtgaacgtgtctctggtcaaacctagcttctacgtgtatagccgggtgaagaacctgaattctagcagggtgcccgacctgctggtggccaccaacttcagcctgctgaaacaggctggcgatgtggaagagaaccctggacctgccgatagcaacggcaccattacagtggaggaactcaaaaagctgctggaacagtggaatcttgtgatcggcttcctgttcctgacctggatctgcctgctgcagttcgcctacgccaaccgcaacagattcctgtacatcatcaaactgatcttcctgtggctgctgtggcccgtgaccctggcttgtttcgtgctggctgctgtttatagaatcaactggatcacaggcggcatcgcaatcgccatggcctgtctggtgggcctgatgtggctgagctacttcatcgccagctttagactgttcgctagaacaagaagcatgtggtcctttaaccccgagacaaacatcctcctgaatgtgccactgcatggcaccatcctgacaagacccctgctggaaagcgagctggtcatcggcgccgtgatcctgcggggccacctgagaatcgctggccaccacctgggcagatgtgacatcaaggacctgcccaaggaaatcactgtggccacaagcagaaccctcagctactacaagctgggagcctctcagagagtggccggcgacagcggcttcgccgcctacagccggtaccggattggcaattacaaactgaacaccgaccacagctccagcagcgacaacatcgctctgctagtgcaggccaccaatttcagcctgctgaagcaagctggagatgtggaagaaaaccccggccctccaaacattaccaacctgtgccccttcggcgaggtgttcaacgccacacggttcgccagcgtgtacgcctggaacagaaagcggatcagcaactgcgtggccgactacagtgtcctgtataactccgccagcttttctacattcaagtgctacggcgtctcccctaccaagctgaacgacctgtgcttcaccaatgtgtacgccgattctttcgtgatcagaggcgacgaggtgcggcagatcgcccctggccagaccggaaagatcgctgattacaactacaagctgcctgatgacttcaccggctgcgtgatcgcctggaactccaacaacctggacagcaaggtggggggcaactacaactacctgtacagactgttcagaaagagcaatctgaagcctttcgagagagatatcagcacagagatctaccaggccggcagcaccccttgtaatggcgttgagggcttcaattgctactttccactgcagagctatggctttcagcctacaaacggcgtgggctaccaaccttacagagtggtggtgctgtctttcgagctgctgcacgcccctggcggaggaggaggcggatctttcatcgaggacctgctgttcaacaaggtgaccctggccgacgccggttttggcggtggcggcggcggctggccttggtacatctggctgggcttcatcgccggactgatcgccatcgtgatggtcaccatcatgctggagggcaggggaagtcttctaacatgcggggacgtggaggaaaatcccggcccagagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaaacggccggctgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 62 - DNA, 1859 bp, synthetic construct
  misc_feature 1..1859: note = expression cassette for VLP
  source 1..1859: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccaccatgtactctttcgtgtctgaggaaaccggcaccctgatcgtgaacagcgtgctgctgtttctggccttcgtggttttcctgctggtcaccctcgccatcctgaccgccctgcggctgtgcgcctactgctgcaacatcgtgaacgtgtctctggtcaaacctagcttctacgtgtatagccgggtgaagaacctgaattctagcagggtgcccgacctgctggtggccaccaacttcagcctgctgaaacaggctggcgatgtggaagagaaccctggacctgccgatagcaacggcaccattacagtggaggaactcaaaaagctgctggaacagtggaatcttgtgatcggcttcctgttcctgacctggatctgcctgctgcagttcgcctacgccaaccgcaacagattcctgtacatcatcaaactgatcttcctgtggctgctgtggcccgtgaccctggcttgtttcgtgctggctgctgtttatagaatcaactggatcacaggcggcatcgcaatcgccatggcctgtctggtgggcctgatgtggctgagctacttcatcgccagctttagactgttcgctagaacaagaagcatgtggtcctttaaccccgagacaaacatcctcctgaatgtgccactgcatggcaccatcctgacaagacccctgctggaaagcgagctggtcatcggcgccgtgatcctgcggggccacctgagaatcgctggccaccacctgggcagatgtgacatcaaggacctgcccaaggaaatcactgtggccacaagcagaaccctcagctactacaagctgggagcctctcagagagtggccggcgacagcggcttcgccgcctacagccggtaccggattggcaattacaaactgaacaccgaccacagctccagcagcgacaacatcgctctgctagtgcaggagggcaggggaagtcttctaacatgcggggacgtggaggaaaatcccggcccaagacccaagctggctagcctcgagtctagagggcccgtttaaacccgctgatcagcctcgaggtaccggatccgcggccgcgatatctctagactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 63 - DNA, 6222 bp, synthetic construct
  misc_feature 1..6222: note = expression vector with expression cassette for VLP
  source 1..6222: mol_type = other DNA
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccaccatgtactctttcgtgtctgaggaaaccggcaccctgatcgtgaacagcgtgctgctgtttctggccttcgtggttttcctgctggtcaccctcgccatcctgaccgccctgcggctgtgcgcctactgctgcaacatcgtgaacgtgtctctggtcaaacctagcttctacgtgtatagccgggtgaagaacctgaattctagcagggtgcccgacctgctggtggccaccaacttcagcctgctgaaacaggctggcgatgtggaagagaaccctggacctatggccgatagcaacggcaccattacagtggaggaactcaaaaagctgctggaacagtggaatcttgtgatcggcttcctgttcctgacctggatctgcctgctgcagttcgcctacgccaaccgcaacagattcctgtacatcatcaaactgatcttcctgtggctgctgtggcccgtgaccctggcttgtttcgtgctggctgctgtttatagaatcaactggatcacaggcggcatcgcaatcgccatggcctgtctggtgggcctgatgtggctgagctacttcatcgccagctttagactgttcgctagaacaagaagcatgtggtcctttaaccccgagacaaacatcctcctgaatgtgccactgcatggcaccatcctgacaagacccctgctggaaagcgagctggtcatcggcgccgtgatcctgcggggccacctgagaatcgctggccaccacctgggcagatgtgacatcaaggacctgcccaaggaaatcactgtggccacaagcagaaccctcagctactacaagctgggagcctctcagagagtggccggcgacagcggcttcgccgcctacagccggtaccggattggcaattacaaactgaacaccgaccacagctccagcagcgacaacatcgctctgctagtgcaggccaccaatttcagcctgctgaagcaagctggagatgtggaagaaaaccccggccctccaaacattaccaacctgtgccccttcggcgaggtgttcaacgccacacggttcgccagcgtgtacgcctggaacagaaagcggatcagcaactgcgtggccgactacagtgtcctgtataactccgccagcttttctacattcaagtgctacggcgtctcccctaccaagctgaacgacctgtgcttcaccaatgtgtacgccgattctttcgtgatcagaggcgacgaggtgcggcagatcgcccctggccagaccggaaagatcgctgattacaactacaagctgcctgatgacttcaccggctgcgtgatcgcctggaactccaacaacctggacagcaaggtggggggcaactacaactacctgtacagactgttcagaaagagcaatctgaagcctttcgagagagatatcagcacagagatctaccaggccggcagcaccccttgtaatggcgttgagggcttcaattgctactttccactgcagagctatggctttcagcctacaaacggcgtgggctaccaaccttacagagtggtggtgctgtctttcgagctgctgcacgcccctggcggaggaggaggcggatctttcatcgaggacctgctgttcaacaaggtgaccctggccgacgccggttttggcggtggcggcggcggctggccttggtacatctggctgggcttcatcgccggactgatcgccatcgtgatggtcaccatcatgctgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttacgcgtggccgctcgagacgcaattcggcttggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 64 - DNA, 20 bp, synthetic construct
  misc_feature 1..20: note = Forward Primer
  source 1..20: mol_type = other DNA
actgctgcaacatcgtgaac

SEQ ID NO: 65 - DNA, 23 bp, synthetic construct
  misc_feature 1..23: note = Reverse Primer
  source 1..23: mol_type = other DNA
tgctagaattcaggttcttcacc

SEQ ID NO: 66 - DNA, 18 bp, synthetic construct
  misc_feature 1..18: note = forward primer
  source 1..18: mol_type = other DNA
ttcctgtggctgctgtgg

SEQ ID NO: 67 - DNA, 20 bp, synthetic construct
  misc_feature 1..20: note = Reverse Primer
  source 1..20: mol_type = other DNA
atgaccagctcgctttccag

SEQ ID NO: 68 - DNA, 22 bp, synthetic construct
  misc_feature 1..22: note = Forward Primer
  source 1..22: mol_type = other DNA
atcagcacagagatctaccagg

SEQ ID NO: 69 - DNA, 20 bp, synthetic construct
  misc_feature 1..20: note = Reverse Primer
  source 1..20: mol_type = other DNA
agcaccaccactctgtaagg

SEQ ID NO: 70 - protein, 65 aa, synthetic construct
  REGION 1..65: note = ACE2 receptor peptide
STIEEQAKTFLDKFNHEAEDLFYQSSLASWNYNTNITEENVQNMNNAGDKWSAFLKEQSTLAQMY

SEQ ID NO: 71 - protein, 15 aa, synthetic construct
  REGION 1..15: note = BAP tag
GLNDIFEAQKIEWHE

SEQ ID NO: 72 - protein, 80 aa, synthetic construct
  REGION 1..80: note = ACE2 receptor peptide with C-terminal BAP tag
STIEEQAKTFLDKFNHEAEDLFYQSSLASWNYNTNITEENVQNMNNAGDKWSAFLKEQSTLAQMYGLNDIFEAQKIEWHE

SEQ ID NO: 73 - DNA, 240 bp, synthetic construct
  misc_feature 1..240: note = ACE2 receptor peptide with C-terminal BAP tag
  source 1..240: mol_type = other DNA
tccactattgaagaacaggcaaagactttcttggacaaattcaaccacgaggccgaagacttgttctatcaaagttcccttgcgagttggaattacaatacgaatatcaccgaagaaaacgttcagaatatgaacaatgcaggcgacaaatggtccgcctttttgaaagaacaaagtaccctggcccagatgtacggtcttaatgacatctttgaagcgcaaaagatcgagtggcacgaa

SEQ ID NO: 74 - protein, 80 aa, synthetic construct
  REGION 1..80: note = ACE2 receptor peptide with N-terminal BAP tag
GLNDIFEAQKIEWHESTIEEQAKTFLDKFNHEAEDLFYQSSLASWNYNTNITEENVQNMNNAGDKWSAFLKEQSTLAQMY

SEQ ID NO: 75 - DNA, 240 bp, synthetic construct
  misc_feature 1..240: note = ACE2 receptor peptide with N-terminal BAP tag
  source 1..240: mol_type = other DNA
ggtcttaatgacatctttgaagcgcaaaagatcgagtggcacgaatccactattgaagaacaggcaaagactttcttggacaaattcaaccacgaggccgaagacttgttctatcaaagttcccttgcgagttggaattacaatacgaatatcaccgaagaaaacgttcagaatatgaacaatgcaggcgacaaatggtccgcctttttgaaagaacaaagtaccctggcccagatgtac

SEQ ID NO: 76 - protein, 9 aa, synthetic construct
  REGION 1..9: note = ACE2 binding peptide
QSYGFQPTN

SEQ ID NO: 77 - protein, 10 aa, synthetic construct
  REGION 1..10: note = ACE2 binding peptide
LQSYGFQPTN

SEQ ID NO: 78 - protein, 13 aa, synthetic construct
  REGION 1..13: note = ACE2 binding peptide
QSYGFQPTNGVGY

SEQ ID NO: 79 - protein, 8 aa, synthetic construct
  REGION 1..8: note = ACE2 binding peptide
QPTNGVGY

SEQ ID NO: 80 - protein, 9 aa, synthetic construct
  REGION 1..9: note = ACE2 binding peptide
FQPTNGVGY

SEQ ID NO: 81 - protein, 4 aa, synthetic construct
  REGION 1..4: note = ACE2 binding peptide
QPTN

SEQ ID NO: 82 - protein, 5 aa, synthetic construct
  REGION 1..5: note = ACE2 binding peptide
FQPTN

SEQ ID NO: 83 - protein, 7 aa, synthetic construct
  REGION 1..7: note = ACE2 binding peptide
FQPTNGV

SEQ ID NO: 84 - protein, 6 aa, synthetic construct
  REGION 1..6: note = ACE2 binding peptide
TNGVGY

SEQ ID NO: 85 - protein, 8 aa, synthetic construct
  REGION 1..8: note = ACE2 binding peptide
FNCYFPLQ

SEQ ID NO: 86 - protein, 9 aa, synthetic construct
  REGION 1..9: note = ACE2 binding peptide
GFNCYFPLQ

SEQ ID NO: 87 - protein, 4 aa, synthetic construct
  REGION 1..4: note = ACE2 binding peptide
EGFN

SEQ ID NO: 88 - protein, 7 aa, synthetic construct
  REGION 1..7: note = ACE2 binding peptide
VEGFNCY

SEQ ID NO: 89 - protein, 10 aa, synthetic construct
  REGION 1..10: note = ACE2 binding peptide
EGFNCYFPLQ

SEQ ID NO: 90 - protein, 5 aa, synthetic construct
  REGION 1..5: note = ACE2 binding peptide
YNYLY

SEQ ID NO: 91 - protein, 7 aa, synthetic construct
  REGION 1..7: note = ACE2 binding peptide
NYNYLYR

SEQ ID NO: 92 - protein, 18 aa, synthetic construct
  REGION 1..18: note = ACE2 binding peptide
SFIEDLLFNKVTLADAGF

SEQ ID NO: 93 - protein, 29 aa, synthetic construct
  REGION 1..29: note = ACE2 binding peptide
SFIEDLLFNKVTLADAGFMKQYGCGKKKK

SEQ ID NO: 94 - protein, 8 aa, synthetic construct
  REGION 1..8: note = ACE2 binding peptide
SFIEDLLF

SEQ ID NO: 95 - protein, 15 aa, synthetic construct
  REGION 1..15: note = ACE2 binding peptide
SFIEDLLFGCGKKKK

SEQ ID NO: 96 - protein, 22 aa, synthetic construct
  REGION 1..22: note = ACE2 binding peptide
SFIEDLLFNKVTLADAGFMKQY

SEQ ID NO: 97 - protein, 15 aa, synthetic construct
  REGION 1..15: note = ACE2 binding peptide
SFIEDAAAGCGKKKK

SEQ ID NO: 98 - protein, 8 aa, synthetic construct
  REGION 1..8: note = ACE2 binding peptide
SFIEDAAA

SEQ ID NO: 99 - protein, 14 aa, synthetic construct
  REGION 1..14: note = ACE2 binding peptide
TRYYYLNYNYTTGY

SEQ ID NO: 100 - protein, 188 aa, synthetic construct
  REGION 1..188: note = ACE2 binding control peptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQ

SEQ ID NO: 101 - DNA, 576 bp, synthetic construct
  misc_feature 1..576: note = immunogenic sequence
  source 1..576: mol_type = other DNA
cctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcacca

SEQ ID NO: 102 - protein, 13 aa, synthetic construct
  REGION 1..13: note = transmembrane domain
WPWYIWLGFIAGL

SEQ ID NO: 103 - DNA, 40 bp, synthetic construct
  misc_feature 1..40: note = transmembrane domain
  source 1..40: mol_type = other DNA
tggccatggtacatttggctaggttttatagctggcttga

SEQ ID NO: 104 - DNA, 3153 bp, synthetic construct
  misc_feature 1..3153: note = bacterial sequence-free vector
  source 1..3153: mol_type = other DNA
cgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccaccatgtactctttcgtgtctgaggaaaccggcaccctgatcgtgaacagcgtgctgctgtttctggccttcgtggttttcctgctggtcaccctcgccatcctgaccgccctgcggctgtgcgcctactgctgcaacatcgtgaacgtgtctctggtcaaacctagcttctacgtgtatagccgggtgaagaacctgaattctagcagggtgcccgacctgctggtggccaccaacttcagcctgctgaaacaggctggcgatgtggaagagaaccctggacctatggccgatagcaacggcaccattacagtggaggaactcaaaaagctgctggaacagtggaatcttgtgatcggcttcctgttcctgacctggatctgcctgctgcagttcgcctacgccaaccgcaacagattcctgtacatcatcaaactgatcttcctgtggctgctgtggcccgtgaccctggcttgtttcgtgctggctgctgtttatagaatcaactggatcacaggcggcatcgcaatcgccatggcctgtctggtgggcctgatgtggctgagctacttcatcgccagctttagactgttcgctagaacaagaagcatgtggtcctttaaccccgagacaaacatcctcctgaatgtgccactgcatggcaccatcctgacaagacccctgctggaaagcgagctggtcatcggcgccgtgatcctgcggggccacctgagaatcgctggccaccacctgggcagatgtgacatcaaggacctgcccaaggaaatcactgtggccacaagcagaaccctcagctactacaagctgggagcctctcagagagtggccggcgacagcggcttcgccgcctacagccggtaccggattggcaattacaaactgaacaccgaccacagctccagcagcgacaacatcgctctgctagtgcaggccaccaatttcagcctgctgaagcaagctggagatgtggaagaaaaccccggccctccaaacattaccaacctgtgccccttcggcgaggtgttcaacgccacacggttcgccagcgtgtacgcctggaacagaaagcggatcagcaactgcgtggccgactacagtgtcctgtataactccgccagcttttctacattcaagtgctacggcgtctcccctaccaagctgaacgacctgtgcttcaccaatgtgtacgccgattctttcgtgatcagaggcgacgaggtgcggcagatcgcccctggccagaccggaaagatcgctgattacaactacaagctgcctgatgacttcaccggctgcgtgatcgcctggaactccaacaacctggacagcaaggtggggggcaactacaactacctgtacagactgttcagaaagagcaatctgaagcctttcgagagagatatcagcacagagatctaccaggccggcagcaccccttgtaatggcgttgagggcttcaattgctactttccactgcagagctatggctttcagcctacaaacggcgtgggctaccaaccttacagagtggtggtgctgtctttcgagctgctgcacgcccctggcggaggaggaggcggatctttcatcgaggacctgctgttcaacaaggtgaccctggccgacgccggttttggcggtggcggcggcggctggccttggtacatctggctgggcttcatcgccggactgatcgccatcgtgatggtcaccatcatgctgtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttacgcgtggccgctcgagacgcaattcggcttggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcg

(19) United States
(12) Patent Application Publication
(10) Pub. No.: US 2023/0140025 A1
(43) Pub. Date: May 4, 2023

(54) Vectors for Producing Virus-Like Particles and Uses Thereof

(71) Applicant: Mediphage Bioceuticals, Inc., Toronto (CA)

(72) Inventors: Roderick SLAVCEV, Waterloo (CA); Nafiseh NAFISSI, Toronto (CA)

(21) Appl. No.: 17/937,234

(22) Filed: Sep. 30, 2022

Related U.S. Application Data
(63) Continuation of application No. PCT/IB2021/052710, filed on Mar. 31, 2021.
(60) Provisional application No. 63/124,397, filed on Dec. 11, 2020.
(60) Provisional application No. 63/003,281, filed on Mar. 31, 2020.

(51) Int. Cl. A61K 39/12; A61P 31/14; A61P 37/04; C07K 14/005; C07K 14/705; C12N 9/48

(52) U.S. Cl. CPC A61K 39/12; A61P 31/14; A61P 37/04; C07K 14/005; C07K 14/705; C12N 9/485; C12Y 304/17023; A61K 2039/5256

(57) ABSTRACT

The present disclosure provides expression vectors and bacterial sequence-free vectors, such as ministring DNA (msDNA), for producing virus-like particles (VLPs) as well as compositions and methods thereof. In some aspects, the methods include treating viral infections in subjects with the vectors, compositions, and VLPs.

### CROSS REFERENCE TO RELATED APPLICATIONS

This application is a continuation of PCT Application No. PCT/IB2021/052710, filed Mar. 31, 2021, which claims the priority benefit of U.S. Provisional Application Nos. 63/124,397, filed Dec. 11, 2020, and 63/003,281, filed Mar. 31, 2020, which are incorporated herein by reference in their entireties.

### REFERENCE TO SEQUENCE LISTING SUBMITTED ELECTRONICALLY

The content of the electronically submitted sequence listing (Name: 4471_0050002_Seqlisting_ST26; Size: 160,205 bytes; and Date of Creation: Sep. 29, 2022) is herein incorporated by reference in its entirety.

### FIELD OF DISCLOSURE

The present disclosure provides vectors for producing virus-like particles (VLPs) and methods of treating subjects with the same.

### BACKGROUND

Despite numerous advances in vaccine technologies, viral infections remain a prevalent health concern that are often under limited control. For example, the COVID-19 coronavirus pandemic became unlike anything the world had seen in over a century, both in terms of global spread and economic impact. It resulted in repeated shutdowns in much of the developed world, with continuously increasing death tolls and new infections.

COVID-19 causes a respiratory infection, along with acute respiratory distress syndrome in severe cases. Pre/asymptomatic airborne transmission and high viral titre early in the course of the disease significantly increase the infectiousness of COVID-19 compared to other coronaviruses such as SARS-CoV, making the development of vaccines critical for management of the pandemic.

VLPs represent potent vaccine candidates that mimic viral physicochemical properties and structure without potentiating viral growth (Cimica, V., & Galarza, J. M., Clin. Immunol. 183: 99-108 (2017)). As such, they confer strong humoral responses, but often limited cell-mediated responses against the ‘whole virus’ as they remain exogenously administered antigens. Furthermore, their production, purification, and storage are costly.

Existing vaccines have often shown limited cross-protection among different viral strains, complicated by the fact that viruses continue to mutate their genomes in response to evolutionary pressures.

There is a need for improved VLPs and methods of treating viral infections.

### BRIEF SUMMARY

The present disclosure is directed to an expression vector comprising: an expression cassette that comprises a nucleic acid sequence encoding a recombinant protein comprising a conserved amino acid sequence from a virus fused to an immunogenic amino acid sequence, a target sequence for a first recombinase flanking each side of the expression cassette, and one or more additional target sequences for one or more additional recombinases integrated within non-binding regions of the target sequence for the first recombinase, wherein protein expressed intracellularly from the expression cassette is capable of forming a virus-like particle (VLP).

In some aspects, the immunogenic amino acid sequence is from the same virus as the conserved amino acid sequence. In some aspects, the conserved amino acid sequence is from a viral glycoprotein. In some aspects, the immunogenic amino acid sequence is from the same viral glycoprotein.

In some aspects, the expression cassette further comprises a nucleic acid sequence encoding a viral envelope protein and/or a nucleic acid sequence encoding a viral matrix protein. In some aspects, the viral envelope protein and/or the viral matrix protein are from the same virus as the conserved amino acid sequence.

In some aspects, the conserved amino acid sequence, the immunogenic amino acid sequence, the viral envelope protein, and/or the viral matrix protein is a consensus sequence.

In some aspects, the recombinant protein is capable of stimulating an immune response against the virus comprising neutralizing antibodies.

In some aspects, the recombinant protein is capable of stimulating a Th1 cell-mediated immune response against the virus.

In some aspects, the immune response is cross-reactive to a related virus or strain.

In some aspects, the recombinant protein excludes amino acid sequences from the virus that stimulate an immune response comprising non-neutralizing antibodies and/or that stimulate a Th2 cell-mediated immune response.

In some aspects, the expression cassette comprises a single open reading frame comprising a nucleic acid sequence encoding a self-cleaving peptide between each nucleic acid sequence encoding a protein.

In some aspects, the virus is a coronavirus, an influenza virus, a human immunodeficiency virus, a human papillomavirus, a hepatitis virus, or an oncolytic virus.

In some aspects, the virus is a coronavirus. In some aspects, the coronavirus is COVID-19.

In some aspects, the expression cassette comprises nucleic acid sequences encoding a coronavirus Membrane (M) protein, a coronavirus Envelope (E) protein, and a recombinant protein comprising a conserved amino acid sequence and an immunogenic amino acid sequence from a coronavirus Spike (S) protein. In some aspects, the conserved amino acid sequence is from the S protein S2′ cleavage site and internal fusion peptide (IFP).

In some aspects, the conserved amino acid sequence comprises SEQ ID NO:12.

In some aspects, the immunogenic amino acid sequence is from the S protein receptor-binding domain (RBD).

In some aspects, the immunogenic amino acid sequence is at least about 90% identical to SEQ ID NO:11.

In some aspects, the recombinant protein further comprises a transmembrane (TM) domain sequence from the S protein.

In some aspects, the recombinant protein excludes amino acid sequences from the S protein that stimulate an immune response comprising non-neutralizing antibodies and/or that stimulate a Th2 cell-mediated immune response.

In some aspects, the amino acid sequence of the recombinant protein is at least about 90% identical to SEQ ID NO:55.

In some aspects, the expression cassette comprises a single open reading frame translated as an amino acid sequence at least about 90% identical to SEQ ID NO:57.

In some aspects, the recombinant protein is capable of stimulating an immune response against COVID-19.

In some aspects, the recombinant protein is capable of stimulating a Th1 cell-mediated immune response against COVID-19.

In some aspects, the immune response is cross-reactive to other coronaviruses. In some aspects, the immune response is cross-reactive to other severe acute respiratory syndrome coronaviruses and/or human betacoronaviruses.

In some aspects, the target sequence for the first recombinase and the one or more additional target sequences for the one or more additional recombinases are selected from the group consisting of the PY54 pal site, the N15 telRL site, the loxP site, φK02 telRL site, the FRT site, the phiC31 attP site, and the λ attP site. In some aspects, the expression vector comprises each of the target sequences. In some aspects, the expression vector comprises the Tel recombinase pal site and the telRL, loxP, and FRT recombinase target binding sequences integrated within the pal site.

In some aspects, the expression vector is for producing a bacterial sequence-free vector. In some aspects, the bacterial sequence-free vector has circular covalently closed ends. In some aspects, the bacterial sequence-free vector has linear covalently closed ends.

In some aspects, the expression vector further comprises at least one enhancer sequence flanking each side of the target sequence for the first recombinase. In some aspects, the at least one enhancer sequence is at least two enhancer sequences. In some aspects, the at least one enhancer sequence is a SV40 enhancer sequence.

The present disclosure is directed to a vector production system comprising recombinant cells designed to encode at least a first recombinase under the control of an inducible promoter, wherein the cells comprise any of the above expression vectors. In some aspects, the inducible promoter is thermally-regulated, chemically-regulated, IPTG regulated, glucose-regulated, arabinose inducible, T7 polymerase regulated, cold-shock inducible, pH inducible, or combinations thereof. In some aspects, the first recombinase is selected from telN and tel, and the expression vector incorporates the target sequence for at least the first recombinase. In some aspects, the recombinant cells have been further designed to encode a nuclease genome editing system, and wherein the expression vector further comprises a backbone sequence containing a cleavage site for the nuclease genome editing system. In some aspects, the nuclease genome editing system is a CRISPR nuclease system comprising a Cas nuclease and gRNA, and the expression vector comprises a target sequence for the gRNA within the backbone sequence.

The present disclosure is directed to a method of producing a bacterial sequence-free vector comprising incubating any of the above vector production systems under suitable conditions for expression of the first recombinase.

The present disclosure is directed to a method of producing a bacterial sequence-free vector comprising incubating any of the above vector production systems that comprise recombinant cells designed to encode a nuclease genome editing system under suitable conditions for expression of the first recombinase and the nuclease genome editing system.

In some aspects, any of the above methods of producing a bacterial sequence-free vector further comprise harvesting the bacterial sequence-free vector.

The present disclosure is directed to a bacterial sequence-free vector produced by any of the above methods of producing a bacterial sequence-free vector.

The present disclosure is directed to a bacterial sequence-free vector comprising an expression cassette that comprises a nucleic acid sequence encoding a recombinant protein comprising a conserved amino acid sequence from a virus fused to an immunogenic amino acid sequence, wherein protein expressed intracellularly from the expression cassette is capable of forming a VLP.

In some aspects, the immunogenic amino acid sequence is from the same virus as the conserved amino acid sequence. In some aspects, the conserved amino acid sequence is from a viral glycoprotein. In some aspects, the immunogenic amino acid sequence is from the same viral glycoprotein.

In some aspects, the expression cassette further comprises a nucleic acid sequence encoding a viral envelope protein and/or a nucleic acid sequence encoding a viral matrix protein. In some aspects, the viral envelope protein and/or the viral matrix protein are from the same virus as the conserved amino acid sequence.

In some aspects, the conserved amino acid sequence, the immunogenic amino acid sequence, the viral envelope protein, and/or the viral matrix protein is a consensus sequence.

In some aspects, the recombinant protein is capable of stimulating an immune response against the virus comprising neutralizing antibodies.

In some aspects, the recombinant protein is capable of stimulating a Th1 cell-mediated immune response against the virus.

In some aspects, the immune response is cross-reactive to a related virus or strain.

In some aspects, the recombinant protein excludes amino acid sequences from the virus that stimulate an immune response comprising non-neutralizing antibodies and/or that stimulate a Th2 cell-mediated immune response.

In some aspects, the expression cassette comprises a single open reading frame comprising a nucleic acid sequence encoding a self-cleaving peptide between each nucleic acid sequence encoding a protein.

In some aspects, the virus is a coronavirus, an influenza virus, a human immunodeficiency virus, a human papillomavirus, a hepatitis virus, or an oncolytic virus.

In some aspects, the virus is a coronavirus. In some aspects, the coronavirus is COVID-19.

In some aspects, the expression cassette comprises nucleic acid sequences encoding a coronavirus M protein, a coronavirus E protein, and a recombinant protein comprising a conserved amino acid sequence and an immunogenic amino acid sequence from a coronavirus S protein. In some aspects, the conserved amino acid sequence is from the S protein S2′ cleavage site and IFP.

In some aspects, the conserved amino acid sequence comprises SEQ ID NO:12.

In some aspects, the immunogenic amino acid sequence is from the S protein RBD.

In some aspects, the immunogenic amino acid sequence is at least about 90% identical to SEQ ID NO:11.

In some aspects, the recombinant protein further comprises a TM domain sequence from the S protein.

In some aspects, the recombinant protein excludes amino acid sequences from the S protein that stimulate an immune response comprising non-neutralizing antibodies and/or that stimulate a Th2 cell-mediated immune response.

In some aspects, the amino acid sequence of the recombinant protein is SEQ ID NO:55.

In some aspects, the expression cassette comprises a single open reading frame translated as an amino acid sequence at least about 90% identical to SEQ ID NO:57.

In some aspects, the recombinant protein is capable of stimulating an immune response against COVID-19.

In some aspects, the recombinant protein is capable of stimulating a Th1 cell-mediated immune response against COVID-19.

In some aspects, the immune response is cross-reactive to other coronaviruses. In some aspects, the immune response is cross-reactive to other severe acute respiratory syndrome coronaviruses and/or human betacoronaviruses.

In some aspects, the bacterial sequence-free vector further comprises at least one enhancer sequence flanking each side of the expression cassette. In some aspects, the at least one enhancer sequence is at least two enhancer sequences. In some aspects, the at least one enhancer sequence is a SV40 enhancer sequence.

In some aspects, the bacterial sequence-free vector comprises circular covalently closed ends.

In some aspects, the bacterial sequence-free vector comprises linear covalently closed ends.

The present disclosure is directed to a polynucleotide encoding an amino acid sequence at least about 90% identical to SEQ ID NO:57.

The present disclosure is directed to a recombinant cell comprising any of the above expression vectors or any of the above bacterial sequence-free vectors.

In some aspects, the present disclosure is directed to a method of producing a VLP, comprising culturing the recombinant cell under suitable conditions for production of the VLP from the expression vector or the bacterial sequence-free vector.

In some aspects, the method of producing a VLP further comprises isolating the VLP. In some aspects, the isolating is by affinity purification. In some aspects, the VLP is produced by any of the above expression vectors or any of the above bacterial sequence-free vectors wherein the virus is a coronavirus. In some aspects, the affinity purification comprises an angiotensin-converting enzyme 2 (ACE2) receptor peptide or an anti-S protein monoclonal antibody. In some aspects, the ACE2 receptor peptide comprises an amino acid sequence that is at least about 90% identical to the amino acid sequence of SEQ ID NO:70. In some aspects, the ACE2 receptor peptide comprises a biotin acceptor peptide (BAP) tag at the C-terminus or N-terminus of the peptide. In some aspects, the BAP tag comprises an amino acid sequence at least about 90% identical to the amino acid sequence of SEQ ID NO:71. In some aspects, the ACE2 receptor peptide or anti-S protein monoclonal antibody is biotinylated and immobilized on a streptavidin-coated bead. In some aspects, the affinity purification comprises microfluidics and/or chromatography. In some aspects, the present disclosure is directed to a VLP produced by any of the methods of producing a VLP.

The present disclosure is directed to a VLP comprising a recombinant protein comprising a conserved amino acid sequence from a virus fused to an immunogenic amino acid sequence. In some aspects, the immunogenic amino acid sequence is from the same virus as the conserved amino acid sequence. In some aspects, the conserved amino acid sequence is from a viral glycoprotein. In some aspects, the immunogenic amino acid sequence is from the same viral glycoprotein.

In some aspects, the VLP further comprises a viral envelope protein and/or a viral matrix protein. In some aspects, the viral envelope protein and/or the viral matrix protein are from the same virus as the conserved amino acid sequence.

In some aspects, the conserved amino acid sequence, the immunogenic amino acid sequence, the viral envelope protein, and/or the viral matrix protein is a consensus sequence.

In some aspects, the recombinant protein is capable of stimulating an immune response against the virus comprising neutralizing antibodies.

In some aspects, the recombinant protein is capable of stimulating a Th1 cell-mediated immune response against the virus.

In some aspects, the immune response is cross-reactive to a related virus or strain.

In some aspects, the recombinant protein excludes amino acid sequences from the virus that stimulate an immune response comprising non-neutralizing antibodies and/or that stimulate a Th2 cell-mediated immune response.

In some aspects, the virus is a coronavirus, an influenza virus, a human immunodeficiency virus, a human papillomavirus, a hepatitis virus, or an oncolytic virus. In some aspects, the virus is a coronavirus.

In some aspects, the coronavirus is COVID-19.

In some aspects, the VLP comprises a coronavirus Membrane (M) protein, a coronavirus Envelope (E) protein, and a recombinant protein comprising a conserved amino acid sequence and an immunogenic amino acid sequence from a coronavirus Spike (S) protein.

In some aspects, the conserved amino acid sequence is from the S protein S2′ cleavage site and internal fusion peptide (IFP).

In some aspects, the conserved amino acid sequence comprises SEQ ID NO:12.

In some aspects, the immunogenic amino acid sequence is from the S protein receptor-binding domain (RBD).

In some aspects, the immunogenic amino acid sequence is at least about 90% identical to SEQ ID NO:11.

In some aspects, the recombinant protein further comprises a transmembrane (TM) domain sequence from the S protein.

In some aspects, the recombinant protein excludes amino acid sequences from the S protein that stimulate an immune response comprising non-neutralizing antibodies and/or that stimulate a Th2 cell-mediated immune response.

In some aspects, the amino acid sequence of the recombinant protein is at least about 90% identical to SEQ ID NO:55.

The present disclosure is directed to a VLP comprising a recombinant protein at least about 90% identical to SEQ ID NO:55, an M protein at least about 90% identical to SEQ ID NO:1, and an E protein at least about 90% identical to SEQ ID NO:3.

In some aspects, the recombinant protein is capable of stimulating an immune response against COVID-19.

In some aspects, the recombinant protein is capable of stimulating a Th1 cell-mediated immune response against COVID-19.

In some aspects, the immune response is cross-reactive to other coronaviruses.

In some aspects, the immune response is cross-reactive to other severe acute respiratory syndrome coronaviruses and/or human betacoronaviruses.

The present disclosure is directed to a composition comprising any of the above expression vectors, any of the above bacterial sequence-free vectors, or any of the above virus-like particles. In some aspects, the composition further comprises a delivery agent. In some aspects, the delivery agent is a nanoparticle. In some aspects, the delivery agent comprises a targeting ligand. In some aspects, the targeting ligand comprises a S protein peptide. In some aspects, the S protein peptide comprises an amino acid sequence at least about 90% identical to any one of SEQ ID NOs:76-99.

The present disclosure is directed to a method of treating a viral infection in a subject, comprising administering to the subject any of the above expression vectors, any of the above bacterial sequence-free vectors, any of the above VLPs, or any of the above compositions, wherein intracellular expression of the expression vector or the bacterial sequence-free vector produces a VLP.

In some aspects, the administering is by parenteral or non-parenteral administration. In some aspects, the administering is by oral, pulmonary, intranasal, intravenous, epidermal, transdermal, subcutaneous, intramuscular, or intraperitoneal administration, or by inhalation.

In some aspects, the VLP stimulates an immune response in the subject comprising neutralizing antibodies against the viral infection.

In some aspects, the VLP stimulates a Th1 cell-mediated immune response in the subject against the viral infection.

In some aspects, the immune response is cross-reactive to a related virus or strain.

In some aspects, the VLP does not stimulate an immune response comprising non-neutralizing antibodies in the subject and/or does not stimulate a Th2 cell-mediated immune response in the subject.

In some aspects, the VLP cross-competes with the infecting virus for binding to a viral receptor.

In some aspects, the VLP cross-competes with a related virus or strain for binding to the viral receptor.

In some aspects, the viral infection is a coronavirus, an influenza virus, a human immunodeficiency virus, a human papillomavirus, a hepatitis virus, or an oncolytic virus.

In some aspects, the viral infection is a coronavirus. In some aspects, the viral infection is COVID-19.

In some aspects, the VLP stimulates an immune response in the subject comprising neutralizing antibodies against COVID-19.

In some aspects, the VLP stimulates a Th1 cell-mediated immune response in the subject against COVID-19.

In some aspects, the immune response is cross-reactive to other coronaviruses.

In some aspects, the immune response is cross-reactive to other severe acute respiratory syndrome coronaviruses and/or human betacoronaviruses.

In some aspects, the VLP does not stimulate an immune response comprising non-neutralizing antibodies in the subject and/or does not stimulate a Th2 cell-mediated immune response in the subject.

In some aspects, the administering is by inhalation.

In some aspects, the VLP cross-competes with COVID-19 for binding to ACE2 receptor, neuropilin-1, or other receptors.

In some aspects, the VLP cross-competes with other coronaviruses for binding to ACE2 receptor, neuropilin-1, and/or other receptors.

In some aspects, the VLP cross-competes with other severe acute respiratory syndrome coronaviruses and/or human betacoronaviruses for binding to ACE2 receptor, neuropilin-1, and/or other receptors.

### BRIEF DESCRIPTION OF THE DRAWINGS

FIG. 1 shows a schematic representation of an exemplary expression cassette for producing a coronavirus VLP containing simian virus 40 enhancers (SV40E); a cytomegalovirus promoter (P_{CMV}); a sequence encoding a coronavirus Envelope (E) protein; a sequence encoding a coronavirus Membrane (M) protein; a sequence encoding a recombinant protein containing sequences from the receptor-binding domain (RBD), the second subunit cleavage domain and internal fusion peptide (S2′IFP), and transmembrane (TM) domain of a coronavirus S protein (referred to herein as a recombinant Spike (S) protein, RBD::S2′IFP::TM); sequences encoding 2A self-cleaving peptides from porcine teschovirus-1 (P2A) to separate the protein-encoding sequences of the expression cassette; and a polyadenylation (pA) signal.

FIG. 2 shows a vector map of an exemplary expression vector (pGL2-SS-CMV-VLP-BGH-SS) containing an expression cassette as described in FIG. 1, in which the pA signal is from bovine growth hormone.

FIG. 3A, FIG. 3B, and FIG. 3C show in vitro expression of genes and protein from the expression vector of FIG. 2. FIG. 3A shows a bar graph depicting relative expression of genes encoding the E protein, M protein, and recombinant S protein (RBD::S2′IFP::TM) as described in FIG. 1 from cells containing the expression vector of FIG. 2 (VLP) as well as control cells without the expression vector (CTL). ***=p<0.001 and ****=p<0.0001. FIG. 3B shows a representative Western blot depicting expression of the recombinant S protein using an antibody that binds to the RBD (α-Spike (RBD)). Detection of beta-actin with the a-beta-actin antibody served as a loading control. Control=protein from cells without the expression vector. VLP=protein from cells containing the expression vector of FIG. 2. FIG. 3C shows the relative mean intensity of recombinant S protein expression from Western blots (n=3) as described for FIG. 3B.

FIG. 4 shows an exemplary msDNA-VLP (msDNA VLP Cov 19-BGH poly) as described herein that is encoded by the expression vector of FIG. 2.

FIG. 5A and FIG. 5B show the concentration (ng/mL) of antibodies that bind to the S1 subunit of the COVID-19 Spike protein (Spike AB) in serum from C57 mice at days 0, 7, 14, 21, 28, 35, 42, and 49 following intramuscular injection with the expression vector of FIG. 2 at day 0 and day 14 (booster). FIG. 5A and FIG. 5B show a line graph and a bar graph of the antibody concentration, respectively.

FIG. 6A and FIG. 6B show a sequence conservation analysis of representative COVID-19 genomes. FIG. 6A shows a bar plot in which the horizontal bars indicate the genomic positions on the x-axis of each of the COVID-19 genes listed on the y-axis as per the Wuhan reference genome (NC_045512.2). FIG. 6B shows a histogram in which bar heights correspond to the percentage of 3928 representative COVID-19 genomes that differed from the Wuhan reference genome at each genomic position.

FIG. 7, FIG. 8A, FIG. 8B, FIG. 8C, and FIG. 8D show histograms in which bar heights correspond to the percentage of analyzed genomes that differed from the Wuhan reference genome at each genomic position, with the analyzed genomes being: (FIG. 7) 3928 representative COVID-19 genomes, 120 severe acute respiratory syndrome coronaviruses (SARS-CoV) genomes, and 257 Middle East respiratory syndrome coronaviruses (MERS-CoV) genomes, (FIG. 8A) 233 COVID-19 genomes of variant strain B.1.1.7, (FIG. 8B) 104 COVID-19 genomes of variant strain B.1.351, (FIG. 8C) 39 COVID-19 genomes of variant strain P.1, and (FIG. 8D) 62 COVID-19 genomes of variant strain B.1.427/429.

FIG. 9 shows an exemplary eukaryotic expression vector (pFastBac™ Dual-VLP) for VLP production in eukaryotic cells as described herein, containing the E, M, and recombinant S proteins as described in FIG. 1.

### DETAILED DESCRIPTION

The present disclosure provides expression vectors and bacterial sequence-free vectors (e.g., ministring DNA (msDNA)) for producing virus-like particles (VLPs), vector production systems, and VLPs, as well as compositions and methods thereof. Some aspects of the present disclosure are directed to treating viral infections in a subject (e.g., coronavirus infections in a human subject, such as COVID-19).

All publications cited herein are hereby incorporated by reference in their entireties, including without limitation all journal articles, books, manuals, patent applications, and patents cited herein, to the same extent as if each individual publication was specifically and individually indicated to be incorporated by reference.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range, or integer range is to be understood to include the value of any integer within the recited range and, when appropriate, fractions thereof (such as one tenth and one hundredth of an integer), unless otherwise indicated. Numeric ranges are inclusive of the numbers defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Systéme International de Unites (SI) accepted form.

Unless otherwise indicated, nucleotide sequences are written left to right in 5′ to 3′ orientation. Amino acid sequences are written left to right in amino to carboxy orientation.

The headings provided herein are not limitations of the various aspects of the disclosure, which can be had by reference to the specification as a whole. Accordingly, the terms defined immediately below are more fully defined by reference to the specification in its entirety.

“Amino acid” is a molecule having the structure wherein a central carbon atom (the alpha-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxyl carbon atom of the carboxylic acid group bonded to the alpha-carbon of one amino acid (or amino acid residue) becomes covalently bound to the amino nitrogen atom of amino group bonded to the non alpha-carbon of an adjacent amino acid. The term “protein” is understood to include the terms “polypeptide” and “peptide” (which, at times may be used interchangeably herein) within its meaning. In addition, proteins comprising multiple polypeptide subunits will also be understood to be included within the meaning of “protein” as used herein. Similarly, fragments of proteins and polypeptides are also within the scope of the disclosure and may be referred to herein as “proteins.” In one aspect of the disclosure, a polypeptide comprises a chimera of two or more parental peptide segments. The term “polypeptide” is also intended to refer to and encompass the products of post-translation modification (“PTM”) of the polypeptide, including without limitation disulfide bond formation, glycosylation, carbamylation, lipidation, acetylation, phosphorylation, amidation, derivatization by known protecting/blocking groups, proteolytic cleavage, modification by non-naturally occurring amino acids, or any other manipulation or modification, such as conjugation with a labeling component. A polypeptide can be derived from a natural biological source or produced by recombinant technology, but is not necessarily translated from a designated nucleic acid sequence. It can be generated in any manner, including by chemical synthesis. An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

“Domain” as used herein can be used interchangeably with the term “peptide segment” and refers to a portion or fragment of a larger polypeptide or protein. A domain need not on its own have functional activity, although in some instances, a domain can have its own biological activity.

“Fused,” “operably linked,” and “operably associated” are used interchangeably herein when referring to two or more domains to broadly refer to any chemical or physical coupling of the two or more domains in the formation of a recombinant polypeptide as disclosed herein. In one embodiment, a recombinant polypeptide as disclosed herein is a chimeric polypeptide comprising a plurality of domains from two or more different polypeptides.

Recombinant polypeptides (i.e., recombinant proteins) comprising two or more domains and/or proteins as disclosed herein can be encoded by a single coding sequence that comprises polynucleotide sequences encoding each domain and/or protein. Unless stated otherwise, the polynucleotide sequences encoding each domain and/or protein are “in frame” such that translation of a single mRNA comprising the polynucleotide sequences results in a single polypeptide comprising each domain and/or protein. Typically, the domains and/or proteins in a recombinant polypeptide as described herein will be fused directly to one another or will be separated by a peptide linker. Various polynucleotide sequences encoding peptide linkers are known in the art and include, for example, self-cleaving peptides.

“Polynucleotide” or “nucleic acid” as used herein refers to a polymeric form of nucleotides. In some instances, a polynucleotide comprises a sequence that is either not immediately contiguous with the coding sequences or is immediately contiguous (on the 5′ end or on the 3′ end) with the coding sequences in the naturally occurring genome of the organism from which it is derived. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA) independent of other sequences. The nucleotides of the disclosure can be ribonucleotides, deoxyribonucleotides, or modified forms of either nucleotide. A polynucleotide as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term polynucleotide encompasses genomic DNA or RNA (depending upon the organism, i.e., RNA genome of viruses), as well as mRNA encoded by the genomic DNA, and cDNA. In certain embodiments, a polynucleotide comprises a conventional phosphodiester bond or a non-conventional bond (e.g., an amide bond, such as found in peptide nucleic acids (PNA)). By “isolated” nucleic acid or polynucleotide is intended a nucleic acid molecule, e.g., DNA or RNA, which has been removed from its native environment. For example, a nucleic acid molecule comprising a polynucleotide encoding a recombinant polypeptide contained in a vector is considered “isolated” for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include polynucleotides and nucleic acids (e.g., nucleic acid molecules) produced synthetically.

As used herein, a “coding region” or “coding sequence” is a portion of a polynucleotide, which consists of codons translatable into amino acids. Although a “stop codon” (TAG, TGA, or TAA) is typically not translated into an amino acid, it may be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino-terminus of the resultant polypeptide, and a translation stop codon at the 3′ terminus, encoding the carboxyl-terminus of the resulting polypeptide.

As used herein, the term “expression control region” refers to a transcription control element that is operably associated with a coding region to direct or control expression of the product encoded by the coding region, including, for example, promoters, enhancers, operators, repressors, ribosome binding sites, translation leader sequences, introns, polyadenylation recognition sequences, RNA processing sites, effector binding sites, stem-loop structures, and transcription termination signals. For example, a coding region and a promoter are “operably associated” (i.e., “operably linked”) if induction of promoter function results in the transcription of mRNA comprising a coding region that encodes the product, and if the nature of the linkage between the promoter and the coding region does not interfere with the ability of the promoter to direct the expression of the product encoded by the coding region or interfere with the ability of the DNA template to be transcribed. Expression control regions include nucleotide sequences located upstream (5′ non-coding sequences), within, or downstream (3′ non-coding sequences) of a coding region, and which influence the transcription, RNA processing, stability, or translation of the associated coding region. If a coding region is intended for expression in a eukaryotic cell, a polyadenylation signal and transcription termination sequence will usually be located 3′ to the coding sequence.

As used herein, the terms “host cell” and “cell” can be used interchangeably and can refer to any type of cell or a population of cells, e.g., a primary cell, a cell in culture, or a cell from a cell line, that harbors or is capable of harboring a nucleic acid molecule (e.g., a recombinant nucleic acid molecule). Host cells can be a prokaryotic cell, or alternatively, the host cells can be eukaryotic, for example, fungal cells, such as yeast cells, and various animal cells, such as insect cells or mammalian cells.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

A “subject” includes any human or nonhuman animal. The term “nonhuman animal” includes, but is not limited to, vertebrates such as mammals, avians, pets, farm animals, nonhuman primates, sheep, cows, goats, pigs, chickens, dogs, cats, and rodents such as mice, rats, and guinea pigs. In preferred aspects, the subject is a human. The terms, “subject” and “patient” are used interchangeably herein.

“Administering” refers to the physical introduction of a therapeutic agent to a subject, using any of the various methods and delivery systems known to those skilled in the art.

The terms “treat,” “treating,” “treatment,” or “therapy” of a subject as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis, such as vaccination).

The term “effective dose” “effective dosage,” or “effective amount” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Various aspects of the disclosure are described in further detail in the following subsections.

### II. Vectors for Producing VLPs

Bacterial sequence-free vectors and their production are described in U.S. Pat. Nos. 9,290,778 and 9,862,954; Nafissi and Slavcev, Microbial Cell Factories 11:154 (2012); and Nafissi et al., Nucleic Acids 3(6):e165 (2014), incorporated by reference herein in their entireties. These bacterial sequence-free vectors are produced from an expression vector (e.g., a plasmid) that contains specialized “Super Sequence” (“SS”) sites comprising target sequences for recombinases. The SS sites flank an expression cassette containing a nucleic acid(s) of interest. When the expression vector is present in a recombinant cell that expresses an appropriate recombinase, bacterial sequence-free vector containing the expression cassette is separated from the backbone DNA of the expression vector. To produce a circular covalently closed (CCC) bacterial sequence-free vector, a production system is used in which the recombinant cell expresses a Cre or Flp recombinase, for example, and the expression vector contains corresponding target sequences for the recombinases. To produce a linear covalently closed (LCC) bacterial sequence-free vector, also referred to herein as a ministring DNA (msDNA), a production system is used in which the recombinant cell expresses a TelN or Tel recombinase, for example, and the expression vector contains corresponding target sequences for the recombinases The bacterial sequence-free vector can then be purified from the cells and used directly as a delivery vector. See U.S. Pat. Nos. 9,290,778 and 9,862,954, Nafissi and Slavcev, and Nafissi et al.

msDNA vectors with LCC ends are torsion-free and not subject to gyrase-directed negative supercoiling during their production in E. coli. Exemplary msDNA vectors carry an expression cassette with a eukaryotic promoter, gene of interest (GOI), intron, and polyA sequence, and nuclear translocation enhancing sequences (Nafissi and Slavcev, and Nafissi et al.). Furthermore, due to its double stranded LCC topology, integration of msDNA into a cell's chromosome causes a chromosomal break, thereby eliminating the cell from the population. Thus, msDNA eliminates any risk of insertional mutagenesis, protecting patients who are administered the msDNA from potential genotoxicity and cancer (Nafissi et al.).

In some aspects, bacterial sequence-free vectors for producing VLPs as disclosed herein include CCC or LCC vectors produced according to any other method known in the art.

### A. Expression Vectors, Expression Cassettes, and Vector Production Systems for Producing Bacterial Sequence-Free Vectors and VLPs

Provided herein is an expression vector comprising: an expression cassette that comprises a nucleic acid sequence encoding a recombinant protein comprising a conserved amino acid sequence from a virus fused to an immunogenic amino acid sequence, wherein protein expressed intracellularly from the expression cassette is capable of forming a VLP.

Provided herein is an expression vector comprising: an expression cassette that comprises a nucleic acid sequence encoding a recombinant protein comprising a conserved amino acid sequence from a virus fused to an immunogenic amino acid sequence, a target sequence for a first recombinase flanking each side of the expression cassette, and one or more additional target sequences for one or more additional recombinases integrated within non-binding regions of the target sequence for the first recombinase, wherein protein expressed intracellularly from the expression cassette is capable of forming a VLP.

Conserved and immunogenic amino acid sequences include those known in the art as well as those determined through known techniques. For example, genome-based reverse vaccinology can be applied towards comparative genomics analysis, a field of biological research that can be used to compare genomic sequences between different pathogenic strains (see, e.g., Sieb et al., Clin. Microbiol. Infect. 18(Suppl. 5):109-116 (2012)). Other sequencing, structural, and computational approaches can also be used (see, e.g., Liljeroos et al., J. Immunol. Res. 2015: 156241; Sette and Rappuoli, Immunity 33(4):530-541 (2010)).

In some aspects, the immunogenic amino acid sequence is from the same virus as the conserved amino acid sequence. In some aspects, the conserved amino acid sequence is from a viral glycoprotein. In some aspects, the immunogenic amino acid sequence is from the same viral glycoprotein.

In some aspects, the expression cassette further comprises a nucleic acid sequence encoding a viral envelope protein and/or a nucleic acid sequence encoding a viral matrix protein. In some aspects, the viral envelope protein and/or the viral matrix protein are from the same virus as the conserved amino acid sequence.

In some aspects, the conserved amino acid sequence, the immunogenic amino acid sequence, the viral envelope protein, and/or the viral matrix protein is a consensus sequence.

In some aspects, the recombinant protein is capable of stimulating an immune response against the virus comprising neutralizing antibodies. Conserved sites, for example, are often recognized by broadly neutralizing antibodies and are susceptible to antibody inactivation (see, e.g., Nabel, N. Engl. J. Med. 368(6): 551-560 (2013)).

In some aspects, the recombinant protein is capable of stimulating a Th1 cell-mediated immune response against the virus. Cell-mediated immunity is the process by which cytotoxic T cells recognize antigen infected cells, to induce cell lysis.

In some aspects, the immune response is cross-reactive to a related virus or strain. For example, conserved sequences among different viral serotypes/strains can be utilized to provide protection against multiple serotypes/strains, including as a universal vaccine.

In some aspects, the recombinant protein excludes amino acid sequences from the virus that stimulate an immune response comprising non-neutralizing antibodies and/or that stimulate a Th2 cell-mediated immune response.

In some aspects, the expression cassette comprises a single open reading frame comprising a nucleic acid sequence encoding a self-cleaving peptide between each nucleic acid sequence encoding a protein such that the translation product of the expression cassette is cleaved intracellularly into two or more proteins. In some aspects, the self-cleaving peptide is a 2A self-cleaving peptide. In some aspects, the 2A self-cleaving peptide is P2A from porcine teschovirus-1. In some aspects, the 2A self-cleaving peptide is T2A from those a asigna virus 2A.

In some aspects, the expression cassette comprises a nucleic acid sequence encoding a self-cleaving peptide between nucleic acid sequences encoding a viral matrix protein and a viral envelope protein, between nucleic acid sequences encoding a viral matrix protein and the recombinant protein, and/or between nucleic acid sequences encoding a viral envelope protein and the recombinant protein. In some aspects, the expression cassette comprises nucleic acid sequences from 5′ to 3′ encoding a viral matrix protein, a self-cleaving peptide, a viral envelope protein, a self-cleaving peptide, and the recombinant protein. In some aspects, the expression cassette comprises nucleic acid sequences from 5′ to 3′ encoding a viral envelope protein, a self-cleaving peptide, a viral matrix protein, a self-cleaving peptide, and the recombinant protein.

In some aspects, the expression cassette further comprises a nucleic acid sequence encoding a marker for gene expression. In some aspects, the marker for gene expression is a fluorescent reporter gene, such as green fluorescent protein (GFP), red fluorescent protein (RFP), yellow fluorescent protein (YFP), or near-infrared fluorescent protein (iRFP); a bioluminescent reporter genes such as luciferase; a selectable antibiotic marker; or LacZ. In some aspects, the expression cassette comprises a nucleic acid sequence encoding a self-cleaving peptide between the nucleic acid sequence encoding a marker for gene expression and any other nucleic acid sequence encoding a protein.

The expression cassette can contain any expression control region known to those of skill in the art operably linked to the protein-encoding nucleic acid sequence(s). In some aspects, the expression control region is a promoter, enhancer, operator, repressor, ribosome binding site, translation leader sequence, intron, polyadenylation recognition sequence, RNA processing site, effector binding site, stem-loop structure, transcription termination signal, or combination thereof.

In some aspects, the target sequence for the first recombinase and the one or more additional target sequences for the one or more additional recombinases are selected from the group consisting of the PY54 pal site, the N15 telRL site, the loxP site, φK02 telRL site, the FRT site, the phiC31 attP site, and the λ attP site. In some aspects, the expression vector comprises each of the target sequences. In some aspects, the expression vector comprises the Tel recombinase pal site and the telRL, loxP, and FRT recombinase target binding sequences integrated within the pal site.

In some aspects, the expression vector is for producing a bacterial sequence-free vector. In some aspects, the bacterial sequence-free vector has circular covalently closed ends. In some aspects, the bacterial sequence-free vector has linear covalently closed ends.

In some aspects, the expression vector further comprises at least one enhancer sequence flanking each side of the target sequence for the first recombinase. In some aspects, the at least one enhancer sequence is at least two enhancer sequences. In some aspects, the at least one enhancer sequence is a SV40 enhancer sequence.

The source of the conserved amino acid sequence, the immunogenic amino acid sequence, and/or a viral protein as disclosed herein can be any virus associated with human or animal infection.

In some aspects, the virus is a coronavirus, an influenza virus, a human immunodeficiency virus, a human papillomavirus, a hepatitis virus, or an oncolytic virus.

In some aspects, the influenza virus is an influenza A virus. In some aspects, the influenza A virus is H1N1, H5N1, or H3N2.

In some aspects, the influenza virus is an influenza B virus.

In some aspects, the coronavirus is a human coronavirus such as, but not limited to, HCoV-229E, HCoV-NL63, HCoV-OC43, HCoV-HKU1, SARS-CoV-1, SARS-CoV-2 (i.e., COVID-19)), and/or MERS-CoV.

In some aspects, the coronavirus is COVID-19 (i.e., Wuhan-Hu-1 or a variant thereof such as, but not limited to, U.K. variant B.1.1.7, South African variant B.1.351, Brazilian variant P.1, or Californian variant B.1.427/429).

Provided herein is a vector production system comprising recombinant cells designed to encode at least a first recombinase under the control of an inducible promoter, wherein the cells comprise an expression vector as disclosed herein comprising a target for the at least first recombinase. In some aspects, the inducible promoter is thermally-regulated, chemically-regulated, IPTG regulated, glucose-regulated, arabinose inducible, T7 polymerase regulated, cold-shock inducible, pH inducible, or combinations thereof. In some aspects, the at least first recombinase is selected from telN and tel, and the expression vector incorporates the target sequence for the at least first recombinase. In some aspects, the at least first recombinase is selected from Cre or Flp, and the expression vector incorporates the target sequence for the at least first recombinase. In some aspects, the recombinant cells have been further designed to encode a nuclease genome editing system, and the expression vector further comprises a backbone sequence containing a cleavage site for the nuclease genome editing system. In some aspects, the nuclease genome editing system is a CRISPR nuclease system comprising a Cas nuclease and gRNA, and the expression vector comprises a target sequence for the gRNA within the backbone sequence.

Provided herein is a method of producing a bacterial sequence-free vector comprising incubating a vector production system as described herein under suitable conditions for expression of the at least first recombinase or the first recombinase and the nuclease genome editing system. In some aspects, the method further comprises harvesting the bacterial sequence-free vector. The present disclosure is also directed to a bacterial sequence-free vector produced by the method.

A.1 Expression Cassettes comprising Coronavirus Sequences

Coronaviruses include any virus of the family Coronaviridae, including the subfamily Coronovirinae, and including the genuses Alphacoronavirus, Betacoronavirus, Gammacoronavirus, and Deltacoronavirus. See, e.g., Fung and Liu (2019). Coronaviruses include human coronaviruses (HCoVs), such as HCoV-229E, HCoV-NL63, HCoV-OC43, HCoV-HKU1, severe acute respiratory syndrome coronaviruses (SARS-CoV, e.g., SARS-CoV-1 and SARS-CoV-2 (i.e., COVID-19)), Middle East respiratory syndrome coronaviruses (MERS-CoV), zoonotic coronaviruses (e.g., SARS-CoVs and MERS-CoVs), bat coronaviruses (BtCoVs), Avian coronavirus, Murine coronavirus, and bulbol coronavirus (BuCoV).

Coronavirus genomes are positive-sense, nonsegmented, single-stranded RNA ranging from about 27 to 32 kilobases (see, e.g., Fung and Liu, Annu. Rev. Microbiol. 73:529-557 (2019)). For example, the complete genome of COVID-19 (also termed Wuhan-Hu-1 coronavirus (WHCV), SARS-CoV-2, and 2019-nCoV) has a size of 29.9 kb, compared to SARS-CoV and MERS-CoV with genomes of 27.9 kb and 30.1 kb, respectively (Zhou et al., Nature 579: 270-273 (2020)). The COVID-19 genome has been found to be 96.2% identical to the Bat CoV RaTG13 genome, which is a type of SARS-CoV-2 found in bats and is likely the source of the virus transmitted to humans via unknown intermediate hosts.

Coronaviruses have a membrane (M) protein, which is the most abundant structural protein that supports the viral envelope and embeds in the envelope with three transmembrane domains. The M protein is essential for virus assembly and budding.

Envelope (E) protein is a small transmembrane protein in coronaviruses that is also present in the envelope at a lower amount than M protein. E protein is also engaged in virus assembly and egress.

The nucleocapsid (N) protein in coronaviruses binds to the RNA genome like beads-on-a-string, forming the helically symmetric nucleocapsid.

The virion surface of coronaviruses is decorated with the trimeric Spike (S) protein. Some betacoronaviruses also have dimeric hemagglutinin-esterase (HE) protein that make up shorter projections on the virion surface. S and HE protein each are type I transmembrane proteins with a large ectodomain and a short endodomain.

The S protein contains two subunits, S1 and S2, and is anchored in the viral envelope at its C-terminus. The S1 subunit of COVID-19, for example, contains the N-terminal domain (NTD) and receptor-binding domain (RBD), while the S2 subunit contains the fusion peptide (FP), internal fusion peptide (IFP), heptad repeat 1/2 (HR1/2), and the transmembrane domain (TM). The S protein's large ectodomain trimerizes and forms the characteristic coronavirus spikes at the virion's surface. The S protein is responsible for receptor binding and virion entry to host cells (Fehr and Perlman, Coronaviruses: An Overview of Their Replication and Pathogenesis. In: Maier H., Bickerton E., Britton P. (eds) Coronaviruses. Methods in Molecular Biology, vol 1282. Humana Press, New York, N.Y.; Wall et al., Cell 180: 1-12 (2020)).

Fusion proteins from many viruses require a proteolytic event near a fusion peptide to enable the pathogen's entry into the target cell. For example, the S protein from COVID-19 possesses two cleavage sites, the first of which sits at the S1/S2 boundary but is not closely linked to the fusion peptide. A second cleavage site (S2′) exposes the internal fusion peptide (IFP), a motif just downstream of S2′ that is highly conserved across all sequenced coronaviruses. The sequence of IFP is SFIEDLLFNKVTLADAGF (SEQ ID NO:7), within which the bolded LLF residues are critical for membrane fusion and infectivity (Madu et al., J. Virol. 83(15): 7411-7421 (2009)). COVID-19 demonstrates the presence of a canonical furin-like cleavage motif at the S1/S2 site not found in other coronaviruses in the same clade, but similarly found in particularly virulent forms of influenza (H5N1). Cleavage via other proteases such as furin at the S1/S2 interface likely widens the tropism of the virus, making animal to human transmission more likely (Coutard et al., Antiviral Res. 176:104742 (2020)).

In some aspects, the expression cassette comprises nucleic acid sequences encoding a coronavirus Membrane (M) protein, a coronavirus Envelope (E) protein, and a recombinant protein comprising a conserved amino acid sequence and an immunogenic amino acid sequence from a coronavirus Spike (S) protein. The M, E, and S proteins can be interchangeably referred to herein as M, E, and S glycoproteins.

In some aspects, the M protein comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:1. In some aspects, the M protein comprises SEQ ID NO:1. In some aspects, the M protein is SEQ ID NO:1.

In some aspects, the nucleic acid sequence encoding the M protein is at least about 90%, at least about 91%, at least about 92%, at least about 93%, at least about 94%, at least about 95%, at least about 96%, at least about 97%, at least about 98%, or at least about 99% identical to SEQ ID NO:2. In some aspects, the nucleic acid sequence encoding the M protein comprises SEQ ID NO:2. In some aspects, the nucleic acid sequence encoding the M protein is SEQ ID NO:2.

In some aspects, the E protein comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:3. In some aspects, the E protein comprises SEQ ID NO:3. In some aspects, the E protein is SEQ ID NO:3. In some aspects, the E protein comprises a replacement of the proline located at amino acid number 71 in SEQ ID NO:3 (i.e., at P71 in SEQ ID NO:3) with another amino acid. In some aspects, the replacement at P71 in SEQ ID NO:3 is a change from proline to leucine (i.e., P71L).

In some aspects, the nucleic acid sequence encoding the E protein is at least about 90%, at least about 91%, at least about 92%, at least about 93%, at least about 94%, at least about 95%, at least about 96%, at least about 97%, at least about 98%, or at least about 99% identical to SEQ ID NO:4. In some aspects, the nucleic acid sequence encoding the E protein comprises SEQ ID NO:4. In some aspects, the nucleic acid sequence encoding the E protein is SEQ ID NO:4. In some aspects, the nucleic acid sequence encoding the E protein comprises a replacement of the codon for proline at nucleotide numbers 211-213 in SEQ ID NO:4 with a codon for another amino acid. In some aspects, the codon for proline at nucleotide numbers 211-213 in SEQ ID NO:4 is replaced with a codon for leucine.

In some aspects, the conserved amino acid sequence is from the S1 subunit or the S2 subunit of the S protein, the RBD of the S protein, the S protein S2′ cleavage site and internal fusion peptide (IFP) of the S protein (referred to herein as STIFP), the M protein, or the E protein.

In some aspects, the conserved amino acid sequence is at least about 90%, at least about 91%, at least about 92%, at least about 93%, at least about 94%, at least about 95%, at least about 96%, at least about 97%, at least about 98%, or at least about 99% identical to any one of SEQ ID NOs:12-54. In some aspects, the conserved amino acid sequence comprises any one of SEQ ID NOs:12-54. In some aspects, the conserved amino acid sequence is any one of SEQ ID NOs:12-54.

In some aspects, the conserved amino acid sequence is at least about 90%, at least about 91%, at least about 92%, at least about 93%, at least about 94%, at least about 95%, at least about 96%, at least about 97%, at least about 98%, or at least about 99% identical to SEQ ID NO:7. In some aspects, the conserved amino acid sequence comprises SEQ ID NO:7. In some aspects, the conserved amino acid sequence is SEQ ID NO:7.

In some aspects, the nucleic acid sequence encoding the conserved amino acid sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:8. In some aspects, the nucleic acid sequence encoding the conserved amino acid sequence of the recombinant protein comprises SEQ ID NO:8. In some aspects, the nucleic acid sequence encoding the conserved amino acid sequence of the recombinant protein is SEQ ID NO:8.

In some aspects, the immunogenic amino acid sequence is from the S protein receptor-binding domain (RBD).

In some aspects, the immunogenic amino acid sequence is at least about 90%, at least about 91%, at least about 92%, at least about 93%, at least about 94%, at least about 95%, at least about 96%, at least about 97%, at least about 98%, or at least about 99% identical to SEQ ID NO:11. In some aspects, the immunogenic amino acid sequence comprises SEQ ID NO:11. In some aspects, the immunogenic amino acid sequence is SEQ ID NO:11. In some aspects, the immunogenic protein comprises a replacement of one or more of: lysine located at amino acid number 88 (i.e., K88), leucine located at amino acid number 123 (i.e., L123), glutamate located at amino acid number 155 (i.e., E155), or asparagine located at amino acid number 172 (i.e., N172) in SEQ ID NO:11 (corresponding to K417, L452, E484, and N501 in SEQ ID NO:5, respectively) with another amino acid. In some aspects, the replacement at K88 is K88N (i.e., a change from lysine to asparagine). In some aspects, the replacement at K88 is K88T (i.e., a change from lysine to threonine). In some aspects, the replacement at L123 is L123R (i.e., a change from leucine to arginine). In some aspects, the replacement at E155 is E155K (i.e., a change from glutamate to lysine). In some aspects, the replacement at N172 is N172Y (i.e., a change from asparagine to tyrosine).

In some aspects, the nucleic acid sequence encoding the immunogenic amino acid sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:101. In some aspects, the nucleic acid sequence encoding the immunogenic amino acid sequence of the recombinant protein comprises SEQ ID NO:101. In some aspects, the nucleic acid sequence encoding the immunogenic amino acid sequence of the recombinant protein is SEQ ID NO:101. In some aspects, the nucleic acid sequence encoding the immunogenic protein comprises a replacement of one or more of: the codon for lysine at nucleotide numbers 262-264 of SEQ ID NO:101 with a codon for another amino acid, the codon for leucine at nucleotide numbers 367-369 of SEQ ID NO:101 with a codon for another amino acid, the codon for glutamate at nucleotide numbers 463-465 of SEQ ID NO:101 with a codon for another amino acid, or the codon for asparagine at nucleotide numbers 514-516 of SEQ ID NO:101 with a codon for another amino acid. In some aspects, the codon for lysine at nucleotide numbers 262-264 is replaced with a codon for asparagine or threonine. In some aspects, the codon for leucine at nucleotide numbers 367-369 is replaced with a codon for arginine. In some aspects, the codon for glutamate at nucleotide numbers 463-465 is replaced with a codon for lysine. In some aspects, the codon for asparagine at nucleotide numbers 514-516 is replaced with a codon for tyrosine.

In some aspects, the recombinant protein further comprises a transmembrane (TM) domain sequence from the S protein.

In some aspects, the TM domain sequence comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:102. In some aspects, the TM domain sequence comprises SEQ ID NO:102. In some aspects, the TM domain sequence is SEQ ID NO:102.

In some aspects, the nucleic acid sequence encoding the TM domain sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:103. In some aspects, the nucleic acid sequence encoding the TM domain sequence of the recombinant protein comprises SEQ ID NO:103. In some aspects, the nucleic acid sequence encoding the TM domain sequence of the recombinant protein is SEQ ID NO:103.

In some aspects, the recombinant protein comprises a conserved amino acid sequence from S2′IFP, an immunogenic amino acid sequence from the RBD, and a TM domain sequence of the S protein.

In some aspects, the recombinant protein excludes amino acid sequences from the S protein that stimulate an immune response comprising non-neutralizing antibodies and/or that stimulate a Th2 cell-mediated immune response.

In some aspects, the amino acid sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:55. In some aspects, the amino acid sequence of the recombinant protein comprises SEQ ID NO:55. In some aspects, the amino acid sequence of the recombinant protein is SEQ ID NO:55. In some aspects, the recombinant protein comprises a replacement of one or more of K88, L123, E155, or N172 in SEQ ID NO:55 with another amino acid. In some aspects, the replacement at K88 is K88N. In some aspects, the replacement at K88 is K88T. In some aspects, the replacement at L123 is L123R. In some aspects, the replacement at E155 is E155K. In some aspects, the replacement at N172 is N172Y.

In some aspects, the nucleic acid sequence encoding the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:56. In some aspects, the nucleic acid sequence encoding the recombinant protein comprises SEQ ID NO:56. In some aspects, the nucleic acid sequence encoding the recombinant protein is SEQ ID NO:56. In some aspects, the nucleic acid sequence encoding the recombinant protein comprises a replacement of one or more of: the codon for lysine at nucleotide numbers 262-264 of SEQ ID NO:56 with a codon for another amino acid, the codon for leucine at nucleotide numbers 367-369 of SEQ ID NO:56 with a codon for another amino acid, the codon for glutamate at nucleotide numbers 463-465 of SEQ ID NO:56 with a codon for another amino acid, or the codon for asparagine at nucleotide numbers 514-516 of SEQ ID NO:56 with a codon for another amino acid. In some aspects, the codon for lysine at nucleotide numbers 262-264 is replaced with a codon for asparagine or threonine. In some aspects, the codon for leucine at nucleotide numbers 367-369 is replaced with a codon for arginine. In some aspects, the codon for glutamate at nucleotide numbers 463-465 is replaced with a codon for lysine. In some aspects, the codon for asparagine at nucleotide numbers 514-516 is replaced with a codon for tyrosine.

In some aspects, the expression cassette comprises a single open reading frame translated as an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:57. In some aspects, the expression cassette comprises a single open reading frame translated as an amino acid sequence comprising SEQ ID NO:57. In some aspects, the expression cassette comprises a single open reading frame translated as an amino acid sequence that is SEQ ID NO:57. In some aspects, the expression cassette comprises a single open reading frame translated as an amino acid sequence that comprises a replacement of one or more of P71, K423, L458, E490, or N507 in SEQ ID NO:57 with another amino acid. In some aspects, the replacement at P71 is P71L. In some aspects, the replacement at K423 is K423N. In some aspects, the replacement at K423 is K423T. In some aspects, the replacement at L458 is L458R. In some aspects, the replacement at E490 is E490K. In some aspects, the replacement at N507 is N507Y.

In some aspects, the expression cassette comprises a single open reading frame that is at least about 90%, at least about 91%, at least about 92%, at least about 93%, at least about 94%, at least about 95%, at least about 96%, at least about 97%, at least about 98%, or at least about 99% identical to SEQ ID NO:58. In some aspects, the expression cassette comprises a single open reading frame that comprises SEQ ID NO:58. In some aspects, the expression cassette comprises a single open reading frame that is SEQ ID NO:58. In some aspects, the expression cassette comprises a single open reading frame that comprises a replacement of one or more of: the codon for proline at nucleotide numbers 211-213 in SEQ ID NO:58 with a codon for another amino acid, the codon for lysine at nucleotide numbers 1267-1269 of SEQ ID NO:58 with a codon for another amino acid, the codon for leucine at nucleotide numbers 1372-1374 of SEQ ID NO:58 with a codon for another amino acid, the codon for glutamate at nucleotide numbers 1468-1470 of SEQ ID NO:58 with a codon for another amino acid, or the codon for asparagine at nucleotide numbers 1519-1521 of SEQ ID NO:58 with a codon for another amino acid. In some aspects, the codon for proline at nucleotide numbers 211-213 in SEQ ID NO:58 is replaced with a codon for leucine. In some aspects, the codon for lysine at nucleotide numbers 1267-1269 is replaced with a codon for asparagine or threonine. In some aspects, the codon for leucine at nucleotide numbers 1372-1374 is replaced with a codon for arginine. In some aspects, the codon for glutamate at nucleotide numbers 1468-1470 is replaced with a codon for lysine. In some aspects, the codon for asparagine at nucleotide numbers 1519-1521 is replaced with a codon for tyrosine.

In some aspects, the expression cassette is at least about 90%, at least about 91%, at least about 92%, at least about 93%, at least about 94%, at least about 95%, at least about 96%, at least about 97%, at least about 98%, or at least about 99% identical to the nucleic acid sequence of any one of SEQ ID NOs:59-62. In some aspects, the expression cassette comprises the nucleic acid sequence of any one of SEQ ID NOs:59-62. In some aspects, the expression cassette is the nucleic acid sequence of any one of SEQ ID NOs:59-62.

In some aspects, the recombinant protein is capable of stimulating an immune response against COVID-19.

In some aspects, the recombinant protein is capable of stimulating a Th1 cell-mediated immune response against COVID-19.

In some aspects, the immune response against COVID-19 is against Wuhan-Hu-1 and/or one or more variants such as, but not limited to, the U.K. variant B.1.1.7, the South African variant B.1.351, the Brazilian variant P.1, or the Californian variant B.1.427/429.

In some aspects, the immune response is cross-reactive to other coronaviruses. In some aspects, the immune response is cross-reactive to other severe acute respiratory syndrome coronaviruses and/or human betacoronaviruses.

Provided herein is a polynucleotide encoding an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:57. In some aspects, the polynucleotide encodes an amino acid sequence comprising SEQ ID NO:57. In some aspects, the polynucleotide encodes an amino acid sequence that is SEQ ID NO:57. In some aspects, the polynucleotide encodes an amino acid sequence that comprises a replacement of one or more of P71, K423, L458, E490, or N507 in SEQ ID NO:57 with another amino acid. In some aspects, the replacement at P71 is P71L. In some aspects, the replacement at K423 is K423N. In some aspects, the replacement at K423 is K423T. In some aspects, the replacement at L458 is L458R. In some aspects, the replacement at E490 is E490K. In some aspects, the replacement at N507 is N507Y.

Provided herein is a polynucleotide comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:58. In some aspects, the polynucleotide comprises SEQ ID NO:58. In some aspects, the polynucleotide is SEQ ID NO:58. In some aspects, the polynucleotide comprising a nucleic acid sequence that comprises a replacement of one or more of: the codon for proline at nucleotide numbers 211-213 in SEQ ID NO:58 with a codon for another amino acid, the codon for lysine at nucleotide numbers 1267-1269 of SEQ ID NO:58 with a codon for another amino acid, the codon for leucine at nucleotide numbers 1372-1374 of SEQ ID NO:58 with a codon for another amino acid, the codon for glutamate at nucleotide numbers 1468-1470 of SEQ ID NO:58 with a codon for another amino acid, or the codon for asparagine at nucleotide numbers 1519-1521 of SEQ ID NO:58 with a codon for another amino acid. In some aspects, the codon for proline at nucleotide numbers 211-213 in SEQ ID NO:58 is replaced with a codon for leucine. In some aspects, the codon for lysine at nucleotide numbers 1267-1269 is replaced with a codon for asparagine or threonine. In some aspects, the codon for leucine at nucleotide numbers 1372-1374 is replaced with a codon for arginine. In some aspects, the codon for glutamate at nucleotide numbers 1468-1470 is replaced with a codon for lysine. In some aspects, the codon for asparagine at nucleotide numbers 1519-1521 is replaced with a codon for tyrosine.

### B. Bacterial Sequence-Free Vectors

A bacterial sequence-free vector of the present disclosure can include any expression cassette of the present disclosure.

Provided herein is a bacterial sequence-free vector comprising an expression cassette that comprises a nucleic acid sequence encoding a recombinant protein comprising a conserved amino acid sequence from a virus fused to an immunogenic amino acid sequence, wherein protein expressed intracellularly from the expression cassette is capable of forming a VLP.

In some aspects, the immunogenic amino acid sequence is from the same virus as the conserved amino acid sequence. In some aspects, the conserved amino acid sequence is from a viral glycoprotein. In some aspects, the immunogenic amino acid sequence is from the same viral glycoprotein.

In some aspects, the expression cassette further comprises a nucleic acid sequence encoding a viral envelope protein and/or a nucleic acid sequence encoding a viral matrix protein. In some aspects, the viral envelope protein and/or the viral matrix protein are from the same virus as the conserved amino acid sequence.

In some aspects, the conserved amino acid sequence, the immunogenic amino acid sequence, the viral envelope protein, and/or the viral matrix protein is a consensus sequence.

In some aspects, the recombinant protein is capable of stimulating an immune response against the virus comprising neutralizing antibodies.

In some aspects, the recombinant protein is capable of stimulating a Th1 cell-mediated immune response against the virus.

In some aspects, the immune response is cross-reactive to a related virus or strain.

In some aspects, the recombinant protein excludes amino acid sequences from the virus that stimulate an immune response comprising non-neutralizing antibodies and/or that stimulate a Th2 cell-mediated immune response.

In some aspects, the expression cassette comprises a single open reading frame comprising a nucleic acid sequence encoding a self-cleaving peptide between each nucleic acid sequence encoding a protein. Expression cassettes and self-cleaving peptides include those discussed above with respect to expression vectors.

In some aspects, the virus is a coronavirus, an influenza virus, a human immunodeficiency virus, a human papillomavirus, a hepatitis virus, or an oncolytic virus.

In some aspects, the influenza virus is an influenza A virus. In some aspects, the influenza A virus is H1N1, H5N1, or H3N2.

In some aspects, the influenza virus is an influenza B virus.

In some aspects, the coronavirus is a human coronavirus such as, but not limited to, HCoV-229E, HCoV-NL63, HCoV-OC43, HCoV-HKU1, SARS-CoV-1, SARS-CoV-2 (i.e., COVID-19)), and/or MERS-CoV.

In some aspects, the coronavirus is COVID-19 (i.e., Wuhan-Hu-1 or a variant thereof such as, but not limited to, U.K. variant B.1.1.7, South African variant B.1.351, Brazilian variant P.1, or Californian variant B.1.427/429).

In some aspects, the expression cassette comprises nucleic acid sequences encoding a coronavirus M protein, a coronavirus E protein, and a recombinant protein comprising a conserved amino acid sequence and an immunogenic amino acid sequence from a coronavirus S protein.

In some aspects, the M protein comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:1. In some aspects, the M protein comprises SEQ ID NO:1. In some aspects, the M protein is SEQ ID NO:1.

In some aspects, the nucleic acid sequence encoding the M protein is at least about 90%, at least about 91%, at least about 92%, at least about 93%, at least about 94%, at least about 95%, at least about 96%, at least about 97%, at least about 98%, or at least about 99% identical to SEQ ID NO:2. In some aspects, the nucleic acid sequence encoding the M protein comprises SEQ ID NO:2. In some aspects, the nucleic acid sequence encoding the M protein is SEQ ID NO:2.

In some aspects, the E protein comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:3. In some aspects, the E protein comprises SEQ ID NO:3. In some aspects, the E protein is SEQ ID NO:3. In some aspects, the E protein comprises a replacement of P71 in SEQ ID NO:3 with another amino acid. In some aspects, the replacement at P71 in SEQ ID NO:3 is P71L.

In some aspects, the nucleic acid sequence encoding the E protein is at least about 90%, at least about 91%, at least about 92%, at least about 93%, at least about 94%, at least about 95%, at least about 96%, at least about 97%, at least about 98%, or at least about 99% identical to SEQ ID NO:4. In some aspects, the nucleic acid sequence encoding the E protein comprises SEQ ID NO:4. In some aspects, the nucleic acid sequence encoding the E protein is SEQ ID NO:4. In some aspects, the nucleic acid sequence encoding the E protein comprises a replacement of the codon for proline at nucleotide numbers 211-213 in SEQ ID NO:4 with a codon for another amino acid. In some aspects, the codon for proline at nucleotide numbers 211-213 in SEQ ID NO:4 is replaced with a codon for leucine.

In some aspects, the conserved amino acid sequence is from the S1 subunit or the S2 subunit of the S protein, the RBD of the S protein, the S protein S2′ cleavage site and internal fusion peptide (IFP) of the S protein (referred to herein as STIFP), the M protein, or the E protein.

In some aspects, the conserved amino acid sequence is at least about 90%, at least about 91%, at least about 92%, at least about 93%, at least about 94%, at least about 95%, at least about 96%, at least about 97%, at least about 98%, or at least about 99% identical to any one of SEQ ID NOs:12-54. In some aspects, the conserved amino acid sequence comprises any one of SEQ ID NOs:12-54. In some aspects, the conserved amino acid sequence is any one of SEQ ID NOs:12-54.

In some aspects, the conserved amino acid sequence is at least about 90%, at least about 91%, at least about 92%, at least about 93%, at least about 94%, at least about 95%, at least about 96%, at least about 97%, at least about 98%, or at least about 99% identical to SEQ ID NO:7. In some aspects, the conserved amino acid sequence comprises SEQ ID NO:7. In some aspects, the conserved amino acid sequence is SEQ ID NO:7.

In some aspects, the nucleic acid sequence encoding the conserved amino acid sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:8. In some aspects, the nucleic acid sequence encoding the conserved amino acid sequence of the recombinant protein comprises SEQ ID NO:8. In some aspects, the nucleic acid sequence encoding the conserved amino acid sequence of the recombinant protein is SEQ ID NO:8.

In some aspects, the immunogenic amino acid sequence is from the S protein receptor-binding domain (RBD).

In some aspects, the immunogenic amino acid sequence is at least about 90%, at least about 91%, at least about 92%, at least about 93%, at least about 94%, at least about 95%, at least about 96%, at least about 97%, at least about 98%, or at least about 99% identical to SEQ ID NO:11. In some aspects, the immunogenic amino acid sequence comprises SEQ ID NO:11. In some aspects, the immunogenic amino acid sequence is SEQ ID NO:11. In some aspects, the immunogenic amino acid sequence comprises a replacement of one or more of: K88, L123, E155, or N172 in SEQ ID NO:11 with another amino acid. In some aspects, the replacement at K88 is K88N . In some aspects, the replacement at K88 is K88T. In some aspects, the replacement at L123 is L123R. In some aspects, the replacement at E155 is E155K. In some aspects, the replacement at N172 is N172Y.

In some aspects, the nucleic acid sequence encoding the immunogenic amino acid sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:101. In some aspects, the nucleic acid sequence encoding the immunogenic amino acid sequence of the recombinant protein comprises SEQ ID NO:101. In some aspects, the nucleic acid sequence encoding the immunogenic amino acid sequence of the recombinant protein is SEQ ID NO:101. In some aspects, the nucleic acid sequence encoding the immunogenic amino acid sequence comprises a replacement of one or more of: the codon for lysine at nucleotide numbers 262-264 of SEQ ID NO:101 with a codon for another amino acid, the codon for leucine at nucleotide numbers 367-369 of SEQ ID NO:101 with a codon for another amino acid, the codon for glutamate at nucleotide numbers 463-465 of SEQ ID NO:101 with a codon for another amino acid, or the codon for asparagine at nucleotide numbers 514-516 of SEQ ID NO:101 with a codon for another amino acid. In some aspects, the codon for lysine at nucleotide numbers 262-264 is replaced with a codon for asparagine or threonine. In some aspects, the codon for leucine at nucleotide numbers 367-369 is replaced with a codon for arginine. In some aspects, the codon for glutamate at nucleotide numbers 463-465 is replaced with a codon for lysine. In some aspects, the codon for asparagine at nucleotide numbers 514-516 is replaced with a codon for tyrosine.

In some aspects, the recombinant protein further comprises a transmembrane (TM) domain sequence from the S protein.

In some aspects, the TM domain sequence comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:102. In some aspects, the TM domain sequence comprises SEQ ID NO:102. In some aspects, the TM domain sequence is SEQ ID NO:102.

In some aspects, the nucleic acid sequence encoding the TM domain sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:103. In some aspects, the nucleic acid sequence encoding the TM domain sequence of the recombinant protein comprises SEQ ID NO:103. In some aspects, the nucleic acid sequence encoding the TM domain sequence of the recombinant protein is SEQ ID NO:103.

In some aspects, the recombinant protein comprises a conserved amino acid sequence from S2′IFP, an immunogenic amino acid sequence from the RBD, and a TM domain sequence of the S protein.

In some aspects, the recombinant protein excludes amino acid sequences from the S protein that stimulate an immune response comprising non-neutralizing antibodies and/or that stimulate a Th2 cell-mediated immune response.

In some aspects, the amino acid sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:55. In some aspects, the amino acid sequence of the recombinant protein comprises SEQ ID NO:55. In some aspects, the amino acid sequence of the recombinant protein is SEQ ID NO:55. In some aspects, the amino acid sequence of the recombinant protein comprises a replacement of one or more of K88, L123, E155, or N172 in SEQ ID NO:55 with another amino acid. In some aspects, the replacement at K88 is K88N. In some aspects, the replacement at K88 is K88T. In some aspects, the replacement at L123 is L123R. In some aspects, the replacement at E155 is E155K. In some aspects, the replacement at N172 is N172Y.

In some aspects, the nucleic acid sequence encoding the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:56. In some aspects, the nucleic acid sequence encoding the recombinant protein comprises SEQ ID NO:56. In some aspects, the nucleic acid sequence encoding the recombinant protein is SEQ ID NO:56. In some aspects, the nucleic acid sequence encoding the recombinant protein comprises a replacement of one or more of: the codon for lysine at nucleotide numbers 262-264 of SEQ ID NO:56 with a codon for another amino acid, the codon for leucine at nucleotide numbers 367-369 of SEQ ID NO:56 with a codon for another amino acid, the codon for glutamate at nucleotide numbers 463-465 of SEQ ID NO:56 with a codon for another amino acid, or the codon for asparagine at nucleotide numbers 514-516 of SEQ ID NO:56 with a codon for another amino acid. In some aspects, the codon for lysine at nucleotide numbers 262-264 is replaced with a codon for asparagine or threonine. In some aspects, the codon for leucine at nucleotide numbers 367-369 is replaced with a codon for arginine. In some aspects, the codon for glutamate at nucleotide numbers 463-465 is replaced with a codon for lysine. In some aspects, the codon for asparagine at nucleotide numbers 514-516 is replaced with a codon for tyrosine.

In some aspects, the expression cassette comprises a single open reading frame translated as an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:57. In some aspects, the expression cassette comprises a single open reading frame translated as an amino acid sequence comprising SEQ ID NO:57. In some aspects, the expression cassette comprises a single open reading frame translated as an amino acid sequence that is SEQ ID NO:57. In some aspects, the expression cassette comprises a single open reading frame translated as an amino acid sequence that comprises a replacement of one or more of P71, K423, L458, E490, or N507 in SEQ ID NO:57 with another amino acid. In some aspects, the replacement at P71 is P71L. In some aspects, the replacement at K423 is K423N. In some aspects, the replacement at K423 is K423T. In some aspects, the replacement at L458 is L458R. In some aspects, the replacement at E490 is E490K. In some aspects, the replacement at N507 is N507Y.

In some aspects, the expression cassette comprises a single open reading frame that is at least about 90%, at least about 91%, at least about 92%, at least about 93%, at least about 94%, at least about 95%, at least about 96%, at least about 97%, at least about 98%, or at least about 99% identical to SEQ ID NO:58. In some aspects, the expression cassette comprises a single open reading frame that comprises SEQ ID NO:58. In some aspects, the expression cassette comprises a single open reading frame that is SEQ ID NO:58. In some aspects, the expression cassette comprises a single open reading frame that comprises a replacement of one or more of: the codon for proline at nucleotide numbers 211-213 in SEQ ID NO:58 with a codon for another amino acid, the codon for lysine at nucleotide numbers 1267-1269 of SEQ ID NO:58 with a codon for another amino acid, the codon for leucine at nucleotide numbers 1372-1374 of SEQ ID NO:58 with a codon for another amino acid, the codon for glutamate at nucleotide numbers 1468-1470 of SEQ ID NO:58 with a codon for another amino acid, or the codon for asparagine at nucleotide numbers 1519-1521 of SEQ ID NO:58 with a codon for another amino acid. In some aspects, the codon for proline at nucleotide numbers 211-213 in SEQ ID NO:58 is replaced with a codon for leucine. In some aspects, the codon for lysine at nucleotide numbers 1267-1269 is replaced with a codon for asparagine or threonine. In some aspects, the codon for leucine at nucleotide numbers 1372-1374 is replaced with a codon for arginine. In some aspects, the codon for glutamate at nucleotide numbers 1468-1470 is replaced with a codon for lysine. In some aspects, the codon for asparagine at nucleotide numbers 1519-1521 is replaced with a codon for tyrosine.

In some aspects, the expression cassette is at least about 90%, at least about 91%, at least about 92%, at least about 93%, at least about 94%, at least about 95%, at least about 96%, at least about 97%, at least about 98%, or at least about 99% identical to any one of SEQ ID NOs:59-62. In some aspects, the expression cassette comprises any one of SEQ ID NOs:59-62. In some aspects, the expression cassette is any one of SEQ ID NOs:59-62.

In some aspects, the recombinant protein is capable of stimulating an immune response against COVID-19.

In some aspects, the recombinant protein is capable of stimulating a Th1 cell-mediated immune response against COVID-19.

In some aspects, the immune response against COVID-19 is against Wuhan-Hu-1 and/or one or more variants such as, but not limited to, the U.K. variant B.1.1.7, the South African variant B.1.351, the Brazilian variant P.1, or the Californian variant B.1.427/429.

In some aspects, the immune response is cross-reactive to other coronaviruses. In some aspects, the immune response is cross-reactive to other severe acute respiratory syndrome coronaviruses and/or human betacoronaviruses.

In some aspects, the bacterial sequence-free vector further comprises at least one enhancer sequence flanking each side of the expression cassette. In some aspects, the at least one enhancer sequence is at least two enhancer sequences. In some aspects, the at least one enhancer sequence is a SV40 enhancer sequence.

In some aspects, the bacterial sequence-free vector comprises circular covalently closed ends.

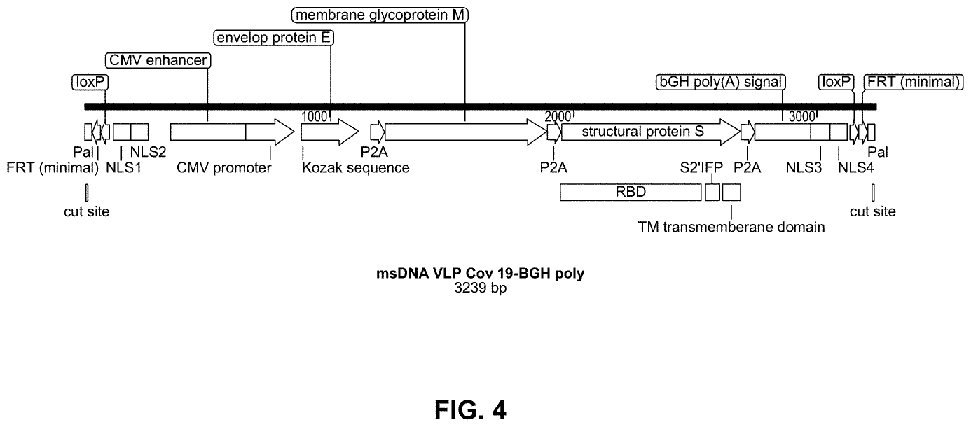

In some aspects, the bacterial sequence-free vector comprises linear covalently closed ends. In some aspects, the bacterial sequence-free vector is a msDNA as disclosed herein. A vector map for an exemplary msDNA is shown in FIG. 4.

In some aspects, the bacterial sequence-free vector is at least about 90%, at least about 91%, at least about 92%, at least about 93%, at least about 94%, at least about 95%, at least about 96%, at least about 97%, at least about 98%, or at least about 99% identical to SEQ ID NO:104. In some aspects, the bacterial sequence-free vector comprises SEQ ID NO:104. In some aspects, the bacterial sequence-free vector is SEQ ID NO:104.

### III. VLPs

In some aspects, a VLP as disclosed herein is produced from the expression cassette of an expression vector and/or the expression cassette of a bacterial sequence-free vector as described herein.

Provided herein is a recombinant cell comprising an expression vector or a bacterial sequence-free vector as described herein.

In some aspects, the recombinant cell is a yeast, bacteria, archaebacteria, fungi, insect, or animal cell, including a mammalian cell. In some aspects, recombinant cells include Drosophila melanogaster cells, Saccharomyces cerevisiae or other yeasts, E. coli, Bacillus subtilis, Sf9 cells, C129 cells, HEK293 cells, Neurospora, BHK, CHO, COS, HeLa cells, Hep G2 cells, and human cells and cell lines.

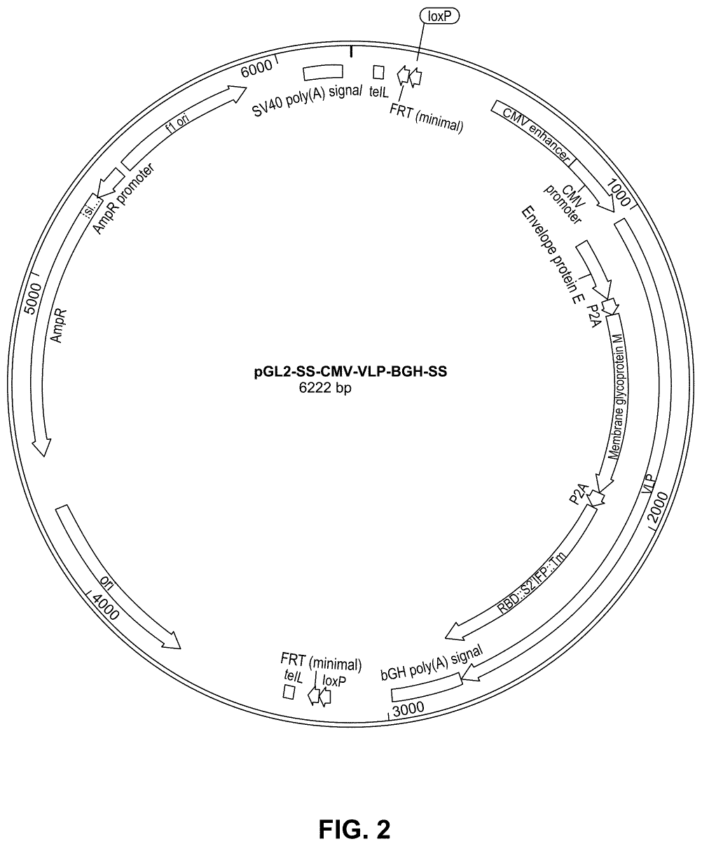

In some aspects, the expression vector is for expression in a human cell or cell line such as the exemplary vector shown in FIG. 2.

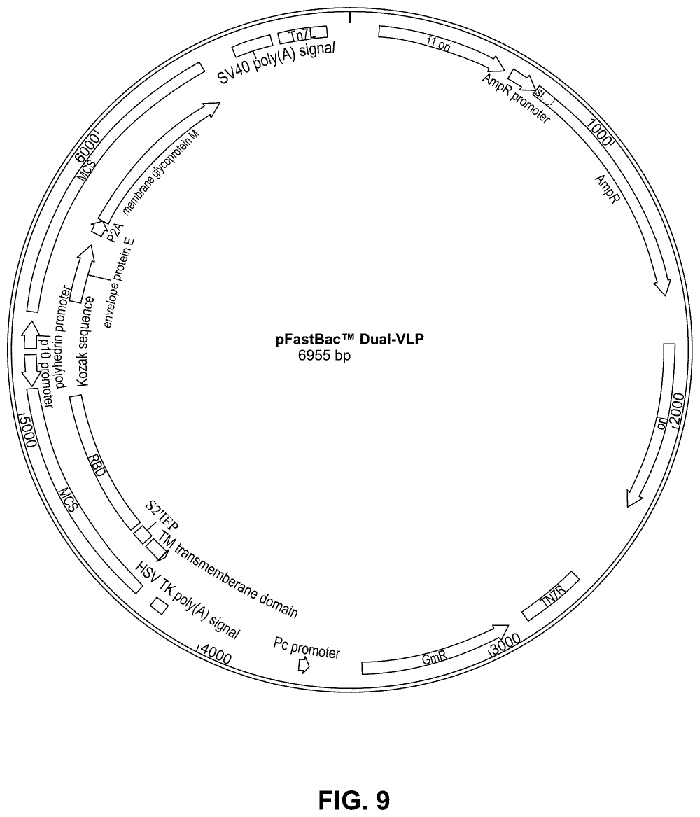

In some aspects, the expression vector is a baculovirus vector such as the exemplary vector shown in FIG. 9 and the cell type is an insect cell (e.g., Sf9 cells).

In some aspects, the present disclosure is directed to a method of producing a VLP, comprising culturing the recombinant cell comprising the expression vector or the bacterial sequence-free vector under suitable conditions for production of the VLP from the expression vector or the bacterial sequence-free vector.

In some aspects, the method of producing a VLP further comprises isolating the VLP. In some aspects, the VLP produced by any of the above expression vectors or any of the above bacterial sequence-free vectors wherein the virus is a coronavirus.

In some aspects, the VLP is isolated from a cell lysate.

In some aspects, the isolating is by affinity purification. In some aspects, the affinity purification comprises microfluidics and/or chromatography.

In some aspects, the affinity purification comprises an angiotensin-converting enzyme 2 (ACE2) receptor peptide or an anti-S protein monoclonal antibody.

In some aspects, the ACE2 receptor peptide comprises an amino acid sequence that is at least about 90%, at least about 91%, at least about 92%, at least about 93%, at least about 94%, at least about 95%, at least about 96%, at least about 97%, at least about 98%, or at least about 99% identical to SEQ ID NO:70. In some aspects, the ACE2 receptor peptide comprises SEQ ID NO:70. In some aspects, the ACE2 receptor peptide is SEQ ID NO:70.

In some aspects, the ACE2 receptor peptide comprises a biotin acceptor peptide (BAP) tag at the C-terminus or N-terminus of the peptide. In some aspects, the BAP tag comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:71. In some aspects, the BAP tag comprises SEQ ID NO:71. In some aspects, the BAP tag is SEQ ID NO:71.

In some aspects, the ACE2 receptor peptide or anti-S protein monoclonal antibody is biotinylated and immobilized on a streptavidin-coated bead. In some aspects, the affinity purification comprises microfluidics and/or chromatography.

In some aspects, the present disclosure is directed to a VLP produced by the method.

Provided herein is a VLP comprising a recombinant protein comprising a conserved amino acid sequence from a virus fused to an immunogenic amino acid sequence.

In some aspects, the immunogenic amino acid sequence is from the same virus as the conserved amino acid sequence.

In some aspects, the conserved amino acid sequence is from a viral glycoprotein. In some aspects, the immunogenic amino acid sequence is from the same viral glycoprotein.

In some aspects, the VLP further comprises a viral envelope protein and/or a viral matrix protein. In some aspects, the viral envelope protein and/or the viral matrix protein are from the same virus as the conserved amino acid sequence.

In some aspects, the conserved amino acid sequence, the immunogenic amino acid sequence, the viral envelope protein, and/or the viral matrix protein is a consensus sequence.

In some aspects, the recombinant protein is capable of stimulating an immune response against the virus comprising neutralizing antibodies.

In some aspects, the recombinant protein is capable of stimulating a Th1 cell-mediated immune response against the virus.

In some aspects, the immune response is cross-reactive to a related virus or strain.

In some aspects, the recombinant protein excludes amino acid sequences from the virus that stimulate an immune response comprising non-neutralizing antibodies and/or that stimulate a Th2 cell-mediated immune response.

In some aspects, the virus is a coronavirus, an influenza virus, a human immunodeficiency virus, a human papillomavirus, a hepatitis virus, or an oncolytic virus.

In some aspects, the influenza virus is an influenza A virus. In some aspects, the influenza A virus is H1N1, H5N1, or H3N2.

In some aspects, the influenza virus is an influenza B virus.

In some aspects, the coronavirus is a human coronavirus such as, but not limited to, HCoV-229E, HCoV-NL63, HCoV-OC43, HCoV-HKU1, SARS-CoV-1, SARS-CoV-2 (i.e., COVID-19)), and/or MERS-CoV.

In some aspects, the coronavirus is COVID-19 (i.e., Wuhan-Hu-1 or a variant thereof such as, but not limited to, U.K. variant B.1.1.7, South African variant B.1.351, Brazilian variant P.1, or Californian variant B.1.427/429).

In some aspects, the VLP comprises a coronavirus M protein, a coronavirus E protein, and a recombinant protein comprising a conserved amino acid sequence and an immunogenic amino acid sequence from a coronavirus S protein.

In some aspects, the M protein comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:1. In some aspects, the M protein comprises SEQ ID NO:1. In some aspects, the M protein is SEQ ID NO:1.

In some aspects, the E protein comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:3. In some aspects, the E protein comprises SEQ ID NO:3. In some aspects, the E protein is SEQ ID NO:3. In some aspects, the E protein comprises a replacement of P71 in SEQ ID NO:3 with another amino acid. In some aspects, the replacement at P71 in SEQ ID NO:3 is P71L.

In some aspects, the conserved amino acid sequence is from the S1 subunit or the S2 subunit of the S protein, the RBD of the S protein, the S protein ST cleavage site and internal fusion peptide (IFP) of the S protein, the M protein, or the E protein.

In some aspects, the conserved amino acid sequence is at least about 90%, at least about 91%, at least about 92%, at least about 93%, at least about 94%, at least about 95%, at least about 96%, at least about 97%, at least about 98%, or at least about 99% identical to any one of SEQ ID NOs:12-54. In some aspects, the conserved amino acid sequence comprises any one of SEQ ID NOs:12-54. In some aspects, the conserved amino acid sequence is any one of SEQ ID NOs:12-54.

In some aspects, the conserved amino acid sequence is at least about 90%, at least about 91%, at least about 92%, at least about 93%, at least about 94%, at least about 95%, at least about 96%, at least about 97%, at least about 98%, or at least about 99% identical to SEQ ID NO:7. In some aspects, the conserved amino acid sequence comprises SEQ ID NO:7. In some aspects, the conserved amino acid sequence is SEQ ID NO:7.

In some aspects, the immunogenic amino acid sequence is from the S protein receptor-binding domain (RBD).

In some aspects, the immunogenic amino acid sequence is at least about 90%, at least about 91%, at least about 92%, at least about 93%, at least about 94%, at least about 95%, at least about 96%, at least about 97%, at least about 98%, or at least about 99% identical to SEQ ID NO:11. In some aspects, the immunogenic amino acid sequence comprises SEQ ID NO:11. In some aspects, the immunogenic amino acid sequence is SEQ ID NO:11. In some aspects, the immunogenic amino acid sequence comprises a replacement of one or more of: K88, L123, E155, or N172 in SEQ ID NO:11 with another amino acid. In some aspects, the replacement at K88 is K88N . In some aspects, the replacement at K88 is K88T. In some aspects, the replacement at L123 is L123R. In some aspects, the replacement at E155 is E155K. In some aspects, the replacement at N172 is N172Y.

In some aspects, the recombinant protein further comprises a transmembrane (TM) domain sequence from the S protein.

In some aspects, the TM domain sequence comprises an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:102. In some aspects, the TM domain sequence comprises SEQ ID NO:102. In some aspects, the TM domain sequence is SEQ ID NO:102.

In some aspects, the recombinant protein comprises a conserved amino acid sequence from S2′IFP, an immunogenic amino acid sequence from the RBD, and a TM domain sequence of the S protein.

In some aspects, the recombinant protein excludes amino acid sequences from the S protein that stimulate an immune response comprising non-neutralizing antibodies and/or that stimulate a Th2 cell-mediated immune response.

In some aspects, the amino acid sequence of the recombinant protein is at least about 90%, at least about 91%, at least about 92%, at least about 93%, at least about 94%, at least about 95%, at least about 96%, at least about 97%, at least about 98%, or at least about 99% identical to SEQ ID NO:55. In some aspects, the amino acid sequence of the recombinant protein comprises SEQ ID NO:55. In some aspects, the amino acid sequence of the recombinant protein is SEQ ID NO:55. In some aspects, the amino acid sequence of the recombinant protein comprises a replacement of one or more of K88, L123, E155, or N172 in SEQ ID NO:55 with another amino acid. In some aspects, the replacement at K88 is K88N. In some aspects, the replacement at K88 is K88T. In some aspects, the replacement at L123 is L123R. In some aspects, the replacement at E155 is E155K. In some aspects, the replacement at N172 is N172Y.

Provided herein is a VLP comprising a recombinant protein at least about 90%, at least about 91%, at least about 92%, at least about 93%, at least about 94%, at least about 95%, at least about 96%, at least about 97%, at least about 98%, or at least about 99% identical to SEQ ID NO:55, an M protein at least about 90%, at least about 91%, at least about 92%, at least about 93%, at least about 94%, at least about 95%, at least about 96%, at least about 97%, at least about 98%, or at least about 99% identical to SEQ ID NO:1, and an E protein at least about 90%, at least about 91%, at least about 92%, at least about 93%, at least about 94%, at least about 95%, at least about 96%, at least about 97%, at least about 98%, or at least about 99% identical to SEQ ID NO:3.

Provided herein is a VLP comprising a recombinant protein that comprises SEQ ID NO:55, an M protein that comprises SEQ ID NO:1, and an E protein that comprises SEQ ID NO:3.

Provided herein is a VLP comprising the recombinant protein of SEQ ID NO:55, the M protein of SEQ ID NO:1, and the E protein of SEQ ID NO:3.

In some aspects, the recombinant protein is capable of stimulating an immune response against COVID-19.

In some aspects, the recombinant protein is capable of stimulating a Th1 cell-mediated immune response against COVID-19.

In some aspects, the immune response against COVID-19 is against Wuhan-Hu-1 and/or one or more variants such as, but not limited to, the U.K. variant B.1.1.7, the South African variant B.1.351, the Brazilian variant P.1, or the Californian variant B.1.427/429

In some aspects, the immune response is cross-reactive to other coronaviruses.

In some aspects, the immune response is cross-reactive to other severe acute respiratory syndrome coronaviruses and/or human betacoronaviruses.

### IV. Compositions

Provided herein is a composition comprising any of the expression vectors, bacterial sequence-free vectors, or VLPs as described herein.

In some aspects, the composition further comprises a physiologically acceptable carrier, excipient, or stabilizer. See, e.g., Remington: The Science and Practice of Pharmacy, 22^{nd} ed. (2013). Acceptable carriers, excipients, or stabilizers can include those that are nontoxic to a subject. In some aspects, the composition or one or more components of the composition are sterile. A sterile component can be prepared, for example, by filtration (e.g., by a sterile filtration membrane) or by irradiation (e.g., by gamma irradiation).

An excipient of the present invention can be described as a “pharmaceutically acceptable” excipient when added to a pharmaceutical composition, meaning that the excipient is a compound, material, composition, salt, and/or dosage form which is, within the scope of sound medical judgment, suitable for contact with the tissues of human beings and animals without excessive toxicity, irritation, allergic response, or other problematic complications over the desired duration of contact commensurate with a reasonable benefit/risk ratio. In some aspects, the term “pharmaceutically acceptable” means approved by a regulatory agency of the Federal or a state government or listed in the U.S. Pharmacopeia or other generally recognized international pharmacopeia for use in animals, and more particularly in humans. Various excipients can be used. In some aspects, the excipient can be, but is not limited to, an alkaline agent, a stabilizer, an antioxidant, an adhesion agent, a separating agent, a coating agent, an exterior phase component, a controlled-release component, a solvent, a surfactant, a humectant, a buffering agent, a filler, an emollient, or combinations thereof. Excipients in addition to those discussed herein can include excipients listed in, though not limited to, Remington: The Science and Practice of Pharmacy, 22^{nd} ed. (2013). Inclusion of an excipient in a particular classification herein (e.g., “solvent”) is intended to illustrate rather than limit the role of the excipient. A particular excipient can fall within multiple classifications.

A pharmaceutical composition of the disclosure is formulated to be compatible with its intended route of administration. Exemplary routes of administration include enteral, topical, parenteral, oral, pulmonary, intranasal, intravenous, epidermal, transdermal, subcutaneous, intramuscular, or intraperitoneal administration, or inhalation. “Parenteral administration” as used herein means modes of administration other than enteral and topical administration, usually by injection or infusion, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrapleural, and intrasternal injection and infusion, as well as in vivo electroporation. In some aspects, the formulation is administered via a non-parenteral route, in some aspects, orally. Other non-parenteral routes include a topical, epidermal, or mucosal route of administration, for example, intranasally, vaginally, rectally, sublingually or topically.

In some aspects, the pharmaceutical composition is lyophilized.

A variety of methods are known in the art and are suitable for introduction of nucleic acids into a cell. Examples include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG), and the like), or cell fusion.

Nanoparticle carriers such as liposomes, micelles, and polymeric nanoparticles have been investigated for improving bioavailability and pharmacokinetic properties of therapeutics via various mechanisms, for example, the enhanced permeability and retention (EPR) effect.

Further improvement can be achieved by conjugation of targeting ligands onto nanoparticles to achieve selective delivery to a target cell. For example, receptor-targeted nanoparticle delivery has been shown to improve therapeutic responses both in vitro and in vivo. Targeting ligands that have been investigated include folate, transferrin, antibodies, peptides, and aptamers. Additionally, multiple functionalities can be incorporated into the design of nanoparticles, e.g., to enable imaging and to trigger intracellular drug release.

In some aspects, the composition further comprises a delivery agent. In some aspects, the delivery agent is a nanoparticle. In some aspects, the delivery agent is selected from the group consisting of liposomes, non-lipid polymeric molecules, endosomes, and any combination thereof.

In some aspects, the delivery agent (e.g., a nanoparticle) comprises a targeting ligand.

In some aspects, the targeting ligand comprises a S protein peptide with binding affinity to the ACE2 receptor (e.g., for delivery of an expression vector, bacterial sequence-free vector, or VLP comprising coronavirus sequences).

In some aspects, the S protein peptide is from a conserved region of the S protein. In some aspects, the length of the S protein peptide is from 3 amino acids to 100 amino acids, including any length or range of lengths therein, such as 3 amino acids to 90, 80, 70, 60, 50, 40, 30, 20, or 10 amino acids.

In some aspects, the S protein peptide comprises an amino acid sequence at least about 90%, at least about 91%, at least about 92%, at least about 93%, at least about 94%, at least about 95%, at least about 96%, at least about 97%, at least about 98%, or at least about 99% identical to any one of SEQ ID NOs:76-99. In some aspects, the S protein peptide comprises any one of SEQ ID NOs:76-99. In some aspects, the S protein peptide is any one of SEQ ID NOs:76-99.

### V. Therapeutic Uses and Methods

The expression vectors, bacterial sequence-free vectors (e.g., msDNA), VLPs, and compositions as described herein can be utilized for prophylactic or therapeutic treatment of a subject in need thereof, including as a vaccine against a viral infection (e.g., a coronavirus infection such as COVID-19) infection or as a treatment for individuals infected with a virus.

Provided herein is a vaccine for a viral infection comprising an expression vector, bacterial sequence-free vector, VLP, or composition as described herein.

Provided herein is a method of treating a viral infection in a subject, comprising administering to the subject an expression vector, bacterial sequence-free vector, VLP, or composition as described herein, wherein intracellular expression of the expression vector or the bacterial sequence-free vector in the subject produces a VLP.

Provided herein is an expression vector, bacterial sequence-free vector, VLP, or composition as described herein for use in treating a viral infection in a subject, wherein intracellular expression of the expression vector or the bacterial sequence-free vector in the subject produces a VLP.

Provided herein is use of an expression vector, bacterial sequence-free vector, VLP, or composition for treating a viral infection in a subject, wherein intracellular expression of the expression vector or the bacterial sequence-free vector in the subject produces a VLP.

Provided herein is use of an expression vector, bacterial sequence-free vector, VLP, or composition for the preparation of a medicament for treating a viral infection in a subject, wherein intracellular expression of the expression vector or the bacterial sequence-free vector in the subject produces a VLP.

The expression vector, bacterial sequence-free vector, or composition can be administered to a subject by any route of administration that is effective in treating the viral infection.

In some aspects, the administering is by enteral, topical, parenteral, oral, pulmonary, intranasal, intravenous, epidermal, transdermal, subcutaneous, intramuscular, or intraperitoneal administration, or inhalation.

In some aspects, the administering is by parenteral or non-parenteral administration.

In some aspects, the parenteral administration is by injection or infusion.

In some aspects, the parenteral administration is by intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrapleural, or intrasternal injection or infusion, or by in vivo electroporation.

In some aspects, the non-parenteral administration is oral, topical, epidermal, mucosal, intranasal, vaginal, rectal, or sublingual.

In some aspects, the administering is by oral, pulmonary, intranasal, intravenous, epidermal, transdermal, subcutaneous, intramuscular, or intraperitoneal administration, or by inhalation.

In some aspects, the administering is by the route of viral infection and transmission.

In some aspects, the route of viral infection and transmission is mucosal.

In some aspects, the administering is by oral, nasal, or pulmonary administration for a respiratory tract infection. In some aspects, the administering is by nasal administration.

Applying the inhalation and intranasal routes of administration provide a powerful opportunity to generate supporting immune responses via lungs and nasopharyngeal-associated lymphoid tissues (NALT) in addition to efficient, targeted, and non-invasive delivery of a VLP as described herein to lower respiratory tract tissue.

In some aspects, the administering is vaginal administration for a sexually transmitted infection.

In some aspects, the administering is by intramuscular, subcutaneous, or intradermal administration where both the site and depth of injection effect the immune response. Intramuscular injection offers a powerful alternative and commonly used technique for vaccine administration, particularly as it is validated and readily re-administered.

Administering can be performed, for example, once, a plurality of times, and/or over one or more extended periods. In some aspects, the administering is one time, two times (e.g., a first administration followed by a second administration about 1, about 2, about 3, about 4 or more weeks later), once about every week, once about every month, once about every 2 months, once about every 3 months, once about every 4 months, once about every 6 months, once about every year, or once about every decade.

The expression cassette as described herein provides a VLP conferring a robust humoral immune response with the benefits of a DNA vaccine for internal processing of intracellular pathogen epitopes for T-cell presentation and cell-mediated immunity. In some aspects, immunodominance is successfully conferred to the conserved amino acid sequence of the recombinant protein, and the vaccine generates universal coronavirus immunity.

In some aspects, VLPs that self-assemble intracellularly from translation products of the expression cassette (whether from the expression vector or a bacterial sequence-free vector as described herein) generate a Th1 cell-mediated response as presented in: 1) an MHC-I context to prime specific cytotoxic T-cell activity against virally infected cells; 2) an MHC-II context in phagocytic antigen presenting cells (APCs) for complementary humoral and cell-mediated support.

In some aspects, intracellular assembly of VLP from the expression cassettes as described herein eliminates potential vaccine-mediated TH2 immunopathology and any associated requirement for adjuvant therapy.

In some aspects, the VLP stimulates an immune response in the subject comprising neutralizing antibodies against the viral infection.

In some aspects, the VLP stimulates a Th1 cell-mediated immune response in the subject against the viral infection.

In some aspects, the immune response is cross-reactive to a related virus or strain.

In some aspects, the VLP does not stimulate an immune response comprising non-neutralizing antibodies in the subject and/or does not stimulate a Th2 cell-mediated immune response in the subject.

In some aspects, the VLP induces antibodies that block viral receptor binding, viral genome uncoating, and/or genome injection.

In some aspects, the VLP cross-competes with the infecting virus for binding to a viral receptor.

In some aspects, the VLP cross-competes with a related virus or strain for binding to the viral receptor.

In some aspects, the viral infection is a coronavirus, an influenza virus, a human immunodeficiency virus, a human papillomavirus, a hepatitis virus, or an oncolytic virus.

In some aspects, the influenza virus is an influenza A virus. In some aspects, the influenza A virus is H1N1, H5N1, or H3N2.

In some aspects, the influenza virus is an influenza B virus.

In some aspects, the coronavirus is a human coronavirus such as, but not limited to, HCoV-229E, HCoV-NL63, HCoV-OC43, HCoV-HKU1, SARS-CoV-1, SARS-CoV-2 (i.e., COVID-19)), and/or MERS-CoV.

In some aspects, the coronavirus is COVID-19 (i.e., Wuhan-Hu-1 or a variant thereof such as, but not limited to, U.K. variant B.1.1.7, South African variant B.1.351, Brazilian variant P.1, or Californian variant B.1.427/429).

In some aspects, the VLP stimulates an immune response in the subject comprising neutralizing antibodies against COVID-19.

In some aspects, the VLP stimulates a Th1 cell-mediated immune response in the subject against COVID-19.

In some aspects, the immune response against COVID-19 is against Wuhan-Hu-1 and/or one or more variants such as, but not limited to, the U.K. variant B.1.1.7, the South African variant B.1.351, the Brazilian variant P.1, or the Californian variant B.1.427/429.

In some aspects, the immune response is cross-reactive to other coronaviruses.

In some aspects, the immune response is cross-reactive to other severe acute respiratory syndrome coronaviruses and/or human betacoronaviruses.

In some aspects, the VLP does not stimulate an immune response comprising non-neutralizing antibodies in the subject and/or does not stimulate a Th2 cell-mediated immune response in the subject.

In some aspects, the administering is by inhalation.

The cellular ligand for COVID-19 and many other coronaviruses is the ACE2 receptor found in the lower respiratory tract of humans, which regulates both cross-species and human-to-human transmission. The ACE2 receptor is bound by the S glycoprotein on the surface of coronavirus that, upon fusion, forms a replication-transcription complex in a double membrane vesicle (Letko et al., Nat. Microbiol. 5(4): 562-569 (2020); Wan et al., J. Virol. 4(7) e00127-20 (2020)). The continuous replication and synthesis of nested sets of subgenomic RNAs encode accessory proteins and structural proteins for the viral particles to bud. This causes the virion-containing vesicles to fuse with plasma membrane ultimately releasing the virus into the host (Fehr and Perlman). Hypertensive patients on adrenergic blocking agents (beta-blockers) to control blood pressure are particularly susceptible to infection as beta blockers stimulate ACE2 receptor over-expression in the respiratory tract facilitating viral binding and infection. Susceptibility has also been noted in patients underlying medical conditions such as COPD, diabetes, and cardiovascular disease (Guan et al., Eur. Resp. Journal, 2000547; DOI: 10.1183/13993003.00547-2020 (2020)).

In some aspects, a VLP against coronavirus (e.g., COVID-19) as described herein not only delivers a therapeutic DNA vaccine, but also competes for available coronavirus receptor sites in respiratory tissue, attenuating further infection.

In some aspects, the extrusion of functional VLPs (expressing surface RBD) from cells further promotes competitive interference for available ACE2 receptors on target cells and promotes interaction with B-cells to ensure a robust neutralizing humoral response.

In some aspects, the S2′IFP domain for presentation exposes the highly conserved site and confers immuno-dominance to the determinant via hapten-carrier response.

In some aspects, the VLP cross-competes with COVID-19 for binding to ACE2 receptor, neuropilin-1, and/or other receptors.

In some aspects, the VLP cross-competes with other coronaviruses for binding to ACE2 receptor, neuropilin-1, and/or other receptors.

In some aspects, the VLP cross-competes with other severe acute respiratory syndrome coronaviruses and/or human betacoronaviruses for binding to ACE2 receptor, neuropilin-1, and/or other receptors.

The following examples are offered by way of illustration and not by way of limitation.

### EXAMPLES

### Example 1

### A. Generation of Expression Vectors for Producing Bacterial Sequence-Free Vectors and VLPs

Four expression vectors were produced by cloning sequences derived from COVID-19 into the multicloning site between two specialized supersequence (SS) sites in a ministring expression vector (Mediphage Bioceuticals, Inc., Toronto, Calif.) as described in U.S. Pat. Nos. 9,290,778 and 9,862,954, incorporated by reference herein in their entireties.

The sequences derived from COVID-19 included sequences encoding Envelope (E) protein (GenBank Accession No. QHD43418.1; SEQ ID NO:3) and Membrane (M) protein (GenBank Accession No. QHD43419.1; SEQ ID NO:1). Additionally, a sequence encoding a recombinant Spike (S) protein was produced that contained a fusion of sequences associated with the receptor-binding domain (RBD), the ST cleavage site and internal fusion peptide (STIFP), and the transmembrane (TM) domain (RBD::S2′IFP::TM; SEQ ID NO:55) of the COVID-19 S protein (GenBank Accession No. QHD43416.1; SEQ ID NO:5). The recombinant S protein was engineered to exclude amino acid sequences from the S protein that stimulate an immune response comprising non-neutralizing antibodies and to exclude amino acid sequences that stimulate a Th2 cell-mediated immune response.

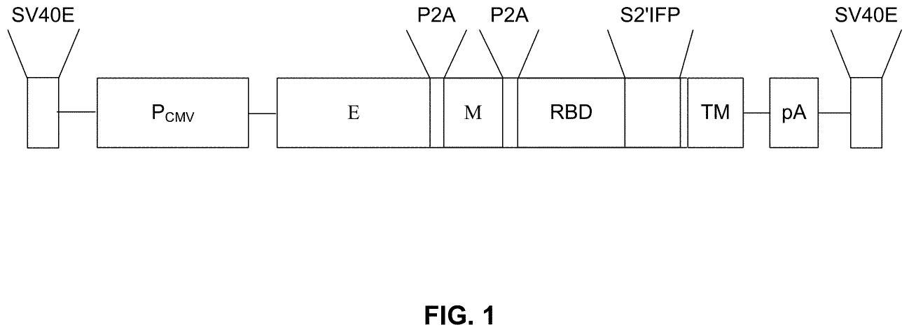

The expression cassettes of three of the expression vectors contained the E protein, the M protein, and the recombinant S protein fused into a single polynucleotide (SEQ ID NO:58) via sequences encoding the self-cleaving peptide P2A from porcine teschovirus-1 2A under the control of a cytomegalovirus (CMV) promoter. FIG. 1 illustrates an exemplary expression cassette.

One of the three expression vectors contained the expression cassette “CMV-E-P2A-M-P2A-RBD::S2′IFP::TM-bGHpolyA” (SEQ ID NO:60), which contained a bovine growth hormone (bGH) polyadenylation (polyA) signal. A map of the expression vector containing the expression cassette is shown in FIG. 2 (pGL2-SS-CMV-VLP-BGH-SS, SEQ ID NO:63).

Another of the three expression vectors contained the expression cassette “CMV-E-P2A-M-P2A-RBD::S2′IFP::TM-SV40polyA” (SEQ ID NO:59), which contained a simian virus 40 (SV40) polyA.

Another of the three expression vectors contained the expression cassette “CMV-E-P2A-M-P2A-RBD::S2′IFP::TM-T2A-GFP-SV40polyA” (SEQ ID NO:61), which contained a green fluorescent protein (GFP) fused to the COVID-19 sequences via a sequence encoding the self-cleaving peptide T2A from those a asigna virus 2A and a SV40 polyA.

A fourth expression vector contained the expression cassette “CMV-E-P2A-M-T2A-MCS-bGHpolyA” (SEQ ID NO:62), which contained a single polynucleotide having the E protein and the M protein fused to one another via a sequence encoding P2A in turn fused to a multiple cloning site (MCS) via a sequence encoding T2A. The expression cassette also contained a CMV promoter and a bGH polyA. The MCS is for insertion of additional sequences, such as recombinant proteins comprising conserved and immunogenic sequences as disclosed herein.

The expression vectors containing the expression cassettes of SEQ ID NOs:59-62 are the same as the expression vector of FIG. 2 and SEQ ID NO:63 except for the different expression cassette.

### B. Expression of COVID-19 Genes

Human lung A549 cells (1×10⁶) were electroporated with 1 μg of the expression vector shown in FIG. 2, or no expression vector. Total RNA was extracted after 48 hours after electroporation and converted to cDNA libraries. 1 μL of cDNA was used as template for Real Time qRT-PCR for E, M, and RBD::S2′IFP::TM transgenes using the gene-specific primers for E, M, and RBD, respectively, shown below in Table 1. Expression of the transgenes was normalized to β-actin expression.

TABLE 1
Primer Sequences

| Gene | Forward Primer | Reverse Primer |
|---|---|---|
| E | ACTGCTGCAACATCGTGAA | TGCTAGAATTCAGGTTCTTC |
| | C (SEQ ID NO: 64) | ACC (SEQ ID NO: 65) |
| M | TTCCTGTGGCTGCTGTGG | ATGACCAGCTCGCTTTCCA |
| | (SEQ ID NO: 66) | G |
| | | (SEQ ID NO: 67) |
| RBD::S2′IFP:: | ATCAGCACAGAGATCTACC | AGCACCACCACTCTGTAAG |
| TM | AGG | G |
| | (SEQ ID NO: 68) | (SEQ ID NO: 69) |

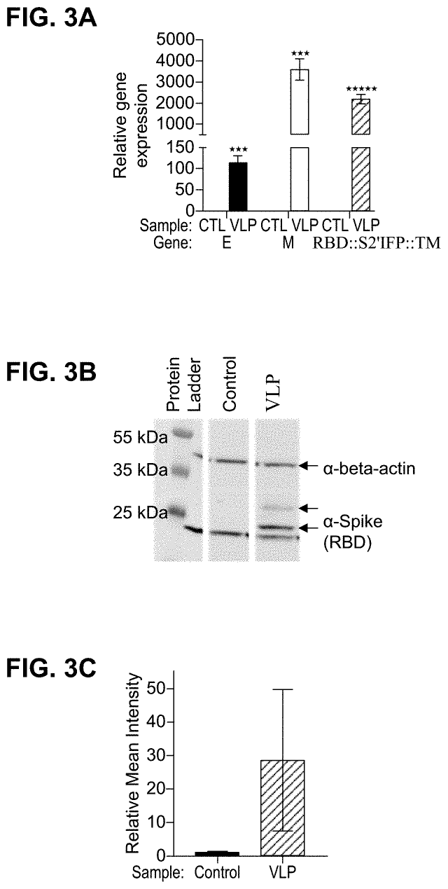

As shown in FIG. 3A, each of the transgenes was detected in cDNA libraries from cells electroporated with the expression vector (“VLP”) but not in cDNA libraries from control cells (“CTL”). The relative gene expression shown in the figure was calculated by ΔΔCT method. Statistical analysis was performed using 1-way ANOVA (***=p<0.001, ****=p<0.0001).

### C. Expression of Recombinant Spike Protein

HEK 293 cells (1×10⁶) were transfected with 2 μg of the expression vector of FIG. 2 using Lipofectamine® 3000 Reagent (Invitrogen). Protein samples were collected 48 hours after transfection. Western blots were prepared by loading 50 μg of whole protein lysate from transfected cells as well as from control cells that were not transfected. A rabbit polyclonal anti-RBD antibody was used to in the detection of recombinant S protein, while a rabbit polyclonal anti-beta-actin antibody was used in the detection of beta-actin as a loading control. An anti-rabbit-horse radish peroxidase (HRP) antibody and chemiluminescence imaging was used for signal detection. A representative Western blot is shown in FIG. 3B, showing that recombinant S protein was detected in protein isolated from cells transfected with the expression vector (“VLP”) but not in protein isolated from control cells.

The relative mean protein intensity of recombinant S protein expression in transfected and control cells was determined by densitometry analysis of Western blot images (n=3). See FIG. 3C.

### Example 2

### Stimulation of Antibody Production by VLP Expression Vectors

The expression vector of FIG. 2 was encapsulated in lipid nanoparticles (Entos Pharmaceuticals) and administered to C57 mice at a dose of 100 μg via intramuscular injection at day 0 followed by a booster dose of 100 μg via intramuscular injection at day 14. Serum was collected via tail vein every 7 days through day 49.

Antibody concentrations in mouse serum were assessed by indirect ELISA by binding to purified S1 protein (Abclonal, Inc.).

Serum was diluted to 1% in PBS and then added to ELISA plates containing the S1 protein. Mouse serum antibodies that bound to the S1 protein were detected by anti-mouse IgG SULFO-TAG™ conjugated antibody (Meso Scale Diagnostics, LLC).

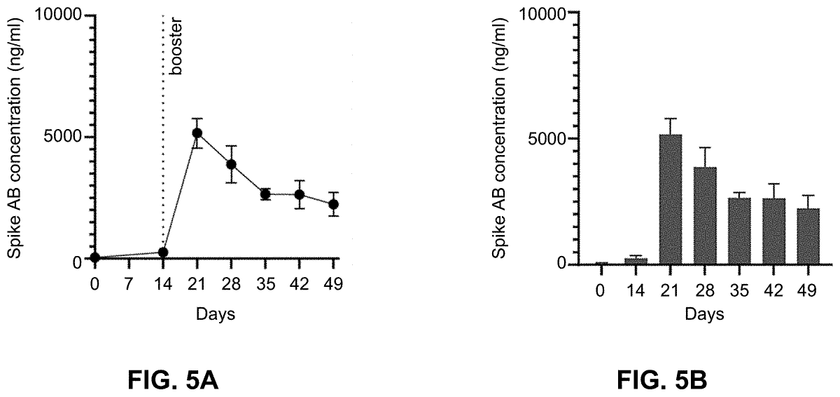

Antibody concentrations are shown in FIGS. 5A and 5B. Concentrations peaked at day 21 at about 5000 ng/mL, with consistent expression maintained at about 3000 ng/mL through day 49.

### Example 3

### A. Characterization of COVID-19 Genomic Sequence Conservation

A total of 3928 representative complete COVID-19 genomes were downloaded from the GISAID database (https://www.gisaid.org). Collection dates for the genomes ranged from December 2019 to February 2021 and contained all major variant strains as well as the Wuhan reference genome (NC_045512.2). Genomes were aligned to the Wuhan reference genome using the MAFFT multiple sequence alignment program. Sequence conservation and nucleotide frequency analyses were performed.

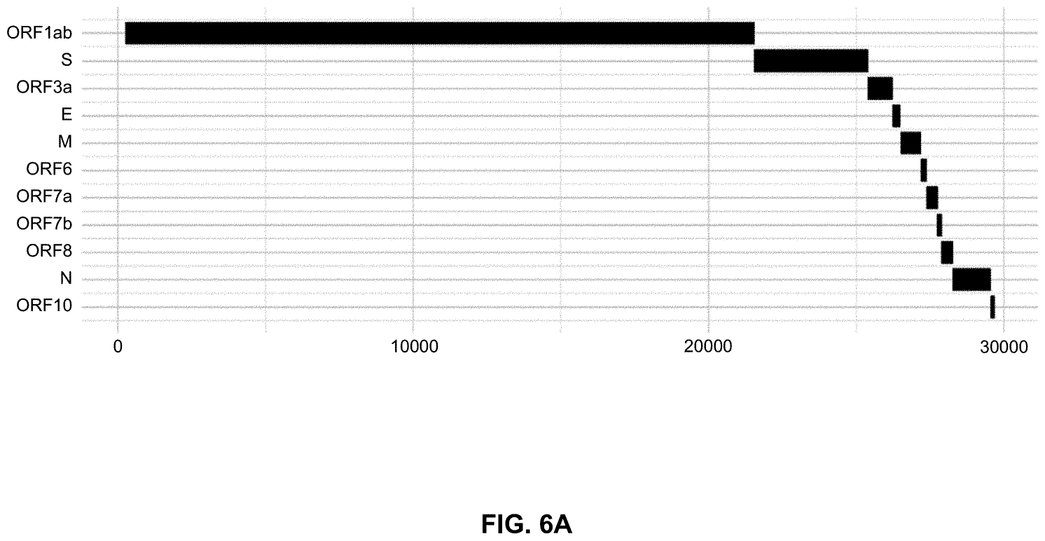

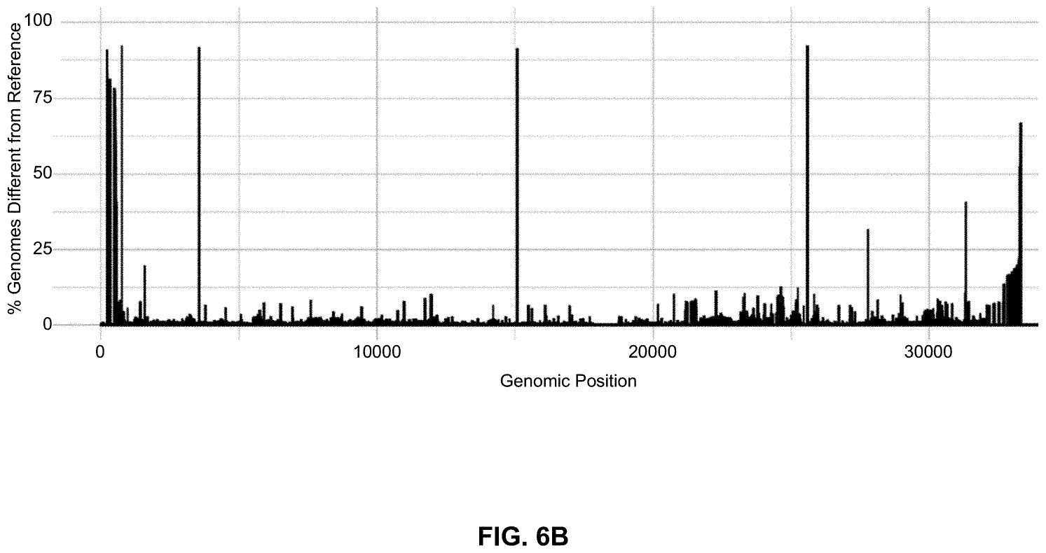

FIG. 6A and FIG. 6B show a sequence conservation analysis of the 3928 representative COVID-19 genomes. FIG. 6A: Horizontal tracks indicate the genomic positions (indicated on the x-axis) of all COVID-19 genes (depicted on the y-axis) as per the Wuhan reference genome. FIG. 6B: The bar heights in the histogram correspond to the percent of genomes that differed from the Wuhan reference genome in each given genomic position. The bar plot and histogram were generated in R version 3.6.1 using the ggplot2 package.

As shown in FIG. 6A and FIG. 6B, the COVID-19 genome has a relatively high level of sequence conservation with few key genomic variants. Ignoring variable 5′ and 3′ end regions, only three genomic positions were found to differ from the reference genome in >50% of sequences. Two of these single nucleotide polymorphisms (SNPs) were found within ORF 1 ab (the first (C241T) in an intergenic region and the second (C14408T→L4715)) within a coding region, and the third (D614G) within the Spike (S) protein.

### B. Characterization of Human Beta Coronavirus Genomic Sequence Conservation

In addition to the 3928 representative complete COVID-19 genomes discussed in part A of this example, 120 SARS-CoV (the virus responsible for SARS) genomes and 257 MERS-CoV (the virus responsible for MERS) genomes were downloaded from the NCBI GenBank® database. Genomes were aligned to the COVID-19 Wuhan reference genome using the MAFFT multiple sequence alignment program. The comparison was possible due to similar genomic organization across these three viral genomes. Sequence conservation and nucleotide frequency analyses were performed.

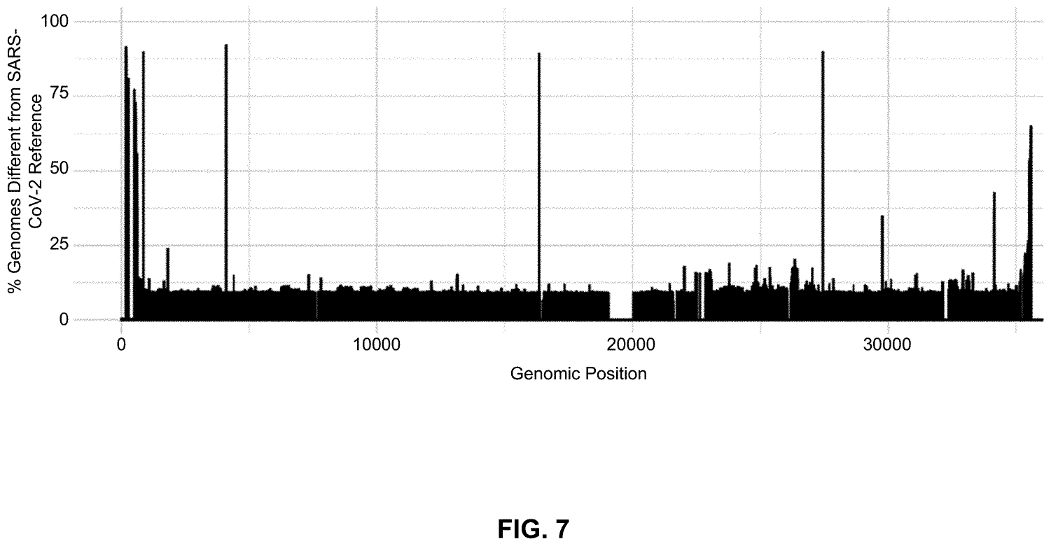

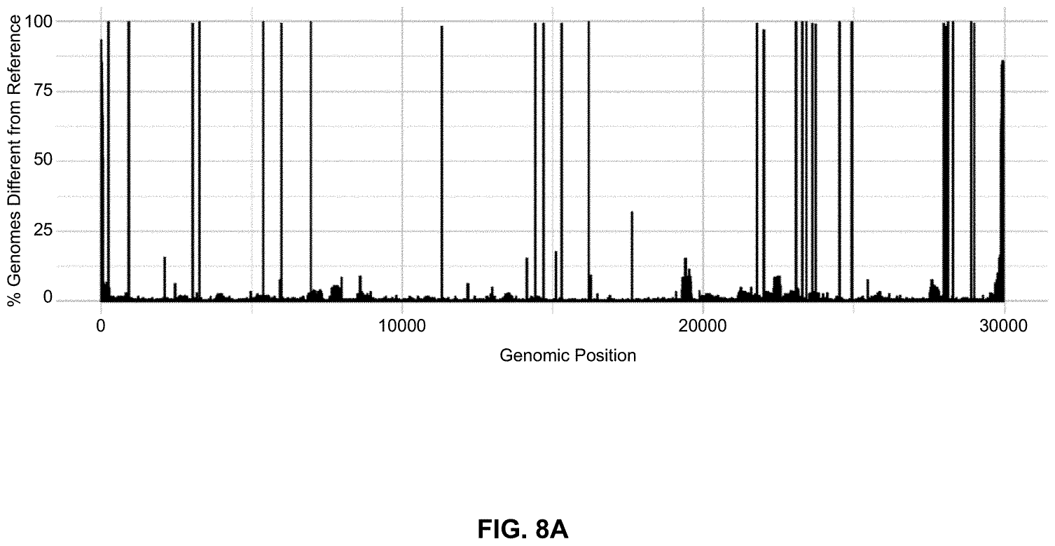

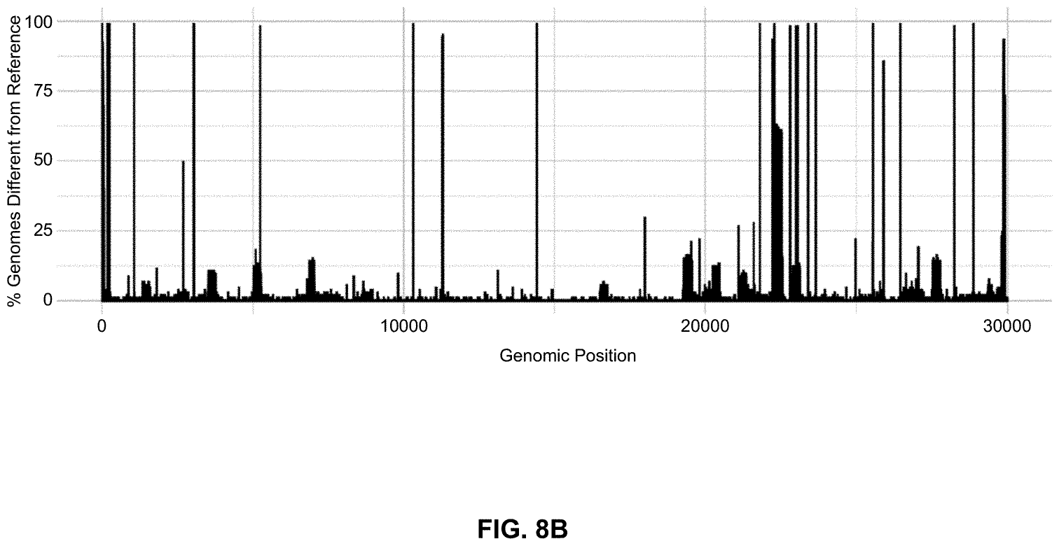

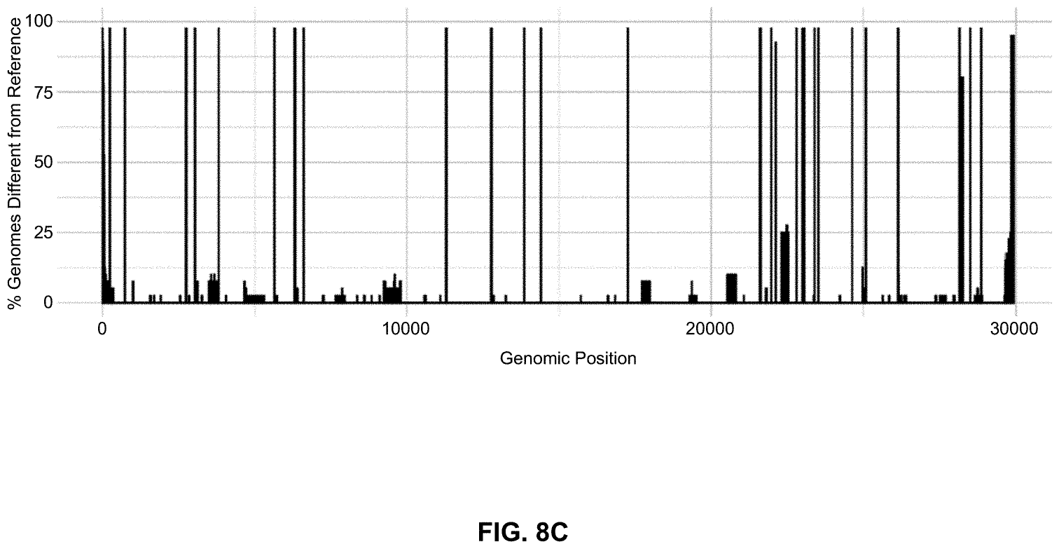

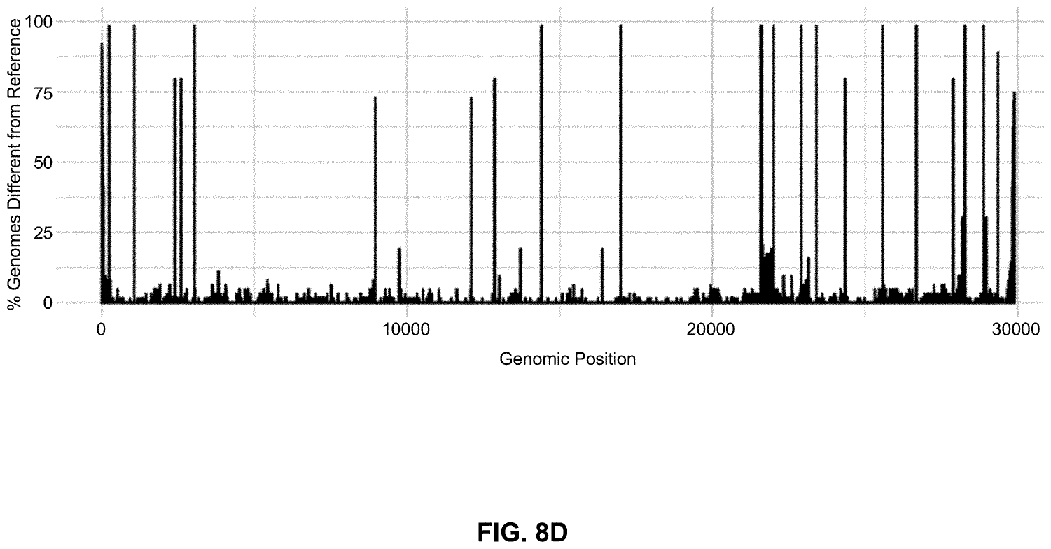

FIG. 7 shows a histogram in which the bar heights correspond to the percent of genomes that differed from the Wuhan reference genome in each given genomic position. The histogram was generated in R version 3.6.1 using the ggplot2 package.

As shown in FIG. 7, the genomes of other prominent human beta coronaviruses (SARS-CoV and MERS-CoV) also have relatively high levels of sequence conservation as compared to the COVID-19 genome.

### C. Identification of Functionally Relevant Mutations in Prominent Variant COVID-19 Strains

The 3928 COVID-19 sequences discussed in part A were filtered for those belonging to key variant strains (U.K. variant B.1.1.7 (n=233), South African variant B.1.351 (n=104), Brazilian variant P.1 (n=39), and Californian variant B.1.427/429 (n=62)). Genomes of the four variant strains were independently aligned to the SARS-CoV-2 Wuhan reference genome (NC_045512.2) using the MAFFT multiple sequence alignment program. Sequence conservation and nucleotide frequency analyses were performed. Functional importance was determined via assessment of BLOSUM 62 matrix score, surface exposure analysis (via PyMol), and literature review.

FIGS. 8A-8D show histograms in which the bar heights correspond to the percent of the variant genomes (B.1.1.7 in FIG. 8A, B.1.351 in FIG. 8B, P.1 in FIG. 8C, and B.1.427/429 in FIG. 8D) that differed from the Wuhan reference genome in each given genomic position. The histograms were generated in R version 3.6.1 using the ggplot2 package.

Table 2 shows a summary of the identified SNPs from variant COVID-19 strains located in regions of the COVID-19 genome contained within the expression cassette shown in FIG. 1.

TABLE 2
Summary of Identified SNPs

| Expression | COVID-19 Variants | | | |
|---|---|---|---|---|
| Cassette | U.K. | South Africa | Brazil | California |
| Sequences | (B.1.1.7) | (B.1.357) | (P.1) | (B.1.427/429) |
| RBD | AAT > TAT | AAT > TAT | AAT > TAT | CTG > CGG |
| | → N501Y | → N501Y | → N501Y | → L452R |
| | | GAA > AAA | GAA > AAA | |
| | | → E484K | → E484K | |
| | | AAG > AAT | AAG > ACG | |
| | | → K417N | → K417T | |
| S2′IFP | — | — | — | — |
| E | — | CCT > CTT | — | — |
| | | → P71L | | |
| M | — | — | — | TTC > TTT |
| | | | | → F53F |

SNPs identified in the receptor-binding domain (RBD) region of the Spike (S) protein of the variant COVID-19 strains were mapped onto a referenced Protein Data Bank (PDB) structure (PBD ID: 6VXX) to assess surface exposure. The N501, K417, and L452 residues were determined to be surface exposed and therefore of potentially greater consequence. The E484 residue was determined not to be surface exposed.

Surface exposure of SNPs identified in the Envelope (E) protein of the variant COVID-19 strains were assessed via structural information in Bianchi et al., BioMed Research International, https://doi.org/10.1155/2020/4389089 (2020). The P71 residue was determined to be surface exposed and therefore of potentially greater consequence.

The SNP identified in the membrane (M) protein results in a synonymous mutation and therefore functional analysis was not performed.

Overall, the analysis showed that sequences selected for the VLP expression cassette as shown in FIG. 1 are relatively robust against COVID-19 variants, especially the S2′IFP site which is completely conserved across all key variant strains as well as in other coronaviruses (SARS-CoV and MERS-CoV).

### Example 4

### A. Generation of Bacterial Sequence-Free Vectors for Producing VLP

DNA ministrings for producing VLP (msDNA-VLP) are produced in inducible E. coli cells from the expression vectors described in Example 1 according to methods described in U.S. Pat. Nos. 9,290,778 and 9,862,954.

msDNA-VLP is purified and concentrated, with quality control testing for purity and sequence.

B. Complexation of Bacterial Sequence-Free Vectors with Nanoparticles

The purified msDNA-VLP and a control msDNA (msDNA-control) expressing a marker protein (e.g., GFP) are complexed with nanoparticles (e.g., lipid nanoparticles (LNPs)). In other studies, commercial LNPs have demonstrated strong transfection efficiency in lung in vivo with msDNA (unpublished data). Commercial LNPs are used as in vitro controls. Commercial JetPEI (https://www.polyplus-transfection.com/products/cgmp-grade-in-vivo-jetpei/) is used as an in vivo control.

The msDNA nanoparticles are lyophilized for in vitro and in vivo tests.

C. In Vitro VLP Formation and Immune Responses from Bacterial Sequence-Free Vectors

The msDNA nanoparticles (i.e., as described in part B of this example) as well as naked msDNA (i.e., msDNA-VLP as described in part A of this example and msDNA-control that are not complexed with nanoparticles) are delivered into a human cell line expressing ACE2 receptors (e.g., A549 cells (ATCC CCL-185)), vascular endothelial cell, or alveolar epithelial cells (Yen, T.-T., et al., Journal of Virology 80(6): 2684-2693 (2006); Qian, Z. et al., American Journal of Respiratory Cell and Molecular Biology 48(6): 742-748 (2013)). Efficiency of the delivery and mean fluorescence are assessed.

Intracellular VLP formation is assessed by transmission electron microscopy.

Cytokine storm and over-activity of inflammation response would be assessed in cell cultures using immune assay techniques.

### D. Production of VLP In Vitro in a Eukaryotic Expression System

A eukaryotic expression vector is produced comprising M-P2A-E and RBD::S2′::TM under control of a promoter for VLP production in eukaryotic cells. An exemplary baculoviral expression vector for VLP production in Sf9 cells is shown in FIG. 9. VLP is produced in vitro and purified using standard techniques.

E. In Vivo VLP Production and Immune Responses from Bacterial Sequence-Free Vectors

The msDNA nanoparticles (i.e., as described in part B of this example) are administered by inhalation, intranasal, or intramuscular routes in an animal model. Cytokine profiles, immunoglobulin profiles, and protective effects against COVID-19 are determined.

For inhalation and intranasal routes, the following administrations are performed: (1) lyophilized msDNA-VLP or msDNA-control nanoparticles are administered by inhalation in one or multiple doses (e.g., dosing at 1, 2, 3, and/or 4 weeks; dosing at 2, 3, 4, 5, 6, 7, 8, 9, 10, 11, and/or 12 months; and/or annual intervals); (2) lyophilized msDNA-VLP or msDNA-control nanoparticles are administered by inhalation in one or multiple doses (e.g., dosing at 1, 2, 3, and/or 4 weeks; dosing at 2, 3, 4, 5, 6, 7, 8, 9, 10, 11, and/or 12 months; and/or annual intervals), followed by intranasal administration of a booster of purified VLP (i.e., as described in part D of this example) in one or multiple doses (e.g., dosing at 1, 2, 3, and/or 4 weeks; dosing at 2, 3, 4, 5, 6, 7, 8, 9, 10, 11, and/or 12 months; and/or annual intervals); or (3) intranasal administration of purified VLP in one or multiple doses (e.g., dosing at 1, 2, 3, and/or 4 weeks; dosing at 2, 3, 4, 5, 6, 7, 8, 9, 10, 11, and/or 12 months; and/or annual intervals).

For intramuscular routes, the following administrations are performed: (1) msDNA-VLP or msDNA-control nanoparticles are administered by injection in one or multiple doses (e.g., dosing at 1, 2, 3, and/or 4 weeks; dosing at 2, 3, 4, 5, 6, 7, 8, 9, 10, 11, and/or 12 months; and/or annual intervals); (2) msDNA-VLP or msDNA-control nanoparticles are administered by injection in one or multiple doses (e.g., dosing at 1, 2, 3, and/or 4 weeks; dosing at 2, 3, 4, 5, 6, 7, 8, 9, 10, 11, and/or 12 months; and/or annual intervals), followed by injection of a booster of purified VLP in one or multiple doses (e.g., dosing at 1, 2, 3, and/or 4 weeks; dosing at 2, 3, 4, 5, 6, 7, 8, 9, 10, 11, and/or 12 months; and/or annual intervals); or (3) injection of a booster of purified VLP in one or multiple doses (e.g., dosing at 1, 2, 3, and/or 4 weeks; dosing at 2, 3, 4, 5, 6, 7, 8, 9, 10, 11, and/or 12 months; and/or annual intervals).

### Example 5

### Affinity Purification of VLPs

A 64-residue ACE2 receptor peptide (“ACE2-64”) was identified as a sufficient interaction interface for binding coronavirus S protein following analysis of four co-crystal structures of S protein and ACE2 receptor as well as one co-crystal structure of lipoprotein E and ACE2 receptor. The amino acid sequence of ACE2-64 is:

(SEQ ID NO: 70)
STIEEQAKTFLDKFNHEAEDLFYQSSLASWNYNTNITEENVQNMNNAGDK
WSAFLKEQSTLAQMY.

The peptide is encoded on an expression plasmid encoding a biotin acceptor peptide (BAP) tag (e.g., GLNDIFEAQKIEWHE (SEQ ID NO:71)) at the C-terminus or N-terminus of ACE2-64 (i.e., SEQ ID NO:72, encoded by SEQ ID NO:73, or SEQ ID NO:74, encoded by SEQ ID NO:75, respectively). The expression plasmid is transformed into a BirA positive E. coli strain, which results in one-step in vivo biotinylation of ACE2-64. The cells are lysed, and the biotinylated ACE2-64 peptides are purified by a commercially available kit and mixed with streptavidin-coated magnetic microbeads.

A commercial monoclonal antibody against the COVID-19 S protein (“S-Ab”) is biotinylated in vitro and mixed with streptavidin-coated magnetic microbeads.

Beads with immobilized ACE2-64 or immobilized S-Ab are washed and equilibrated in an inert Tris buffer (e.g., 20 mM Tris pH 8.0, 150 mM NaCl).

Recombinant cells expressing VLPs from msDNA-VLPs, such as the eukaryotic cells of Example 2(D), are lysed.

Beads with immobilized ACE2-64 or immobilized S-Ab and the cell lysate containing VLPs are added to a microfluidic device and mixed. VLPs captured by the ACE2-64 or S-Ab coated beads are separated from the cell lysate. The beads are then washed three times with a buffer of moderate salinity (e.g., 20 mM Tris pH 8.0, 300 mM NaCl). The VLPs are then purified in a buffer of high salinity (e.g., 20 mM Tris pH 8.0, 1.5 M NaCl), which results in the dissociation of VLPs from the beads. The purified VLPs are collected. Quality control assays, such as agarose gel electrophoresis to detect RNA and episomal DNA, qPCR to assess gDNA levels, and electron microscopy, are performed to confirm the identity and purity of the VLPs.

### Example 6

### Production of Targeting Ligands for Nanoparticle Formulations

A peptide library is derived from the conserved regions of coronavirus S protein and produced by peptide synthesis. Exemplary peptides are SEQ ID NOs:76-99.

Recombinant ACE2 protein is purchased from a commercial source.

The following portion of the COVID-19 S protein is provided as a control for binding to ACE2, with the bolded and underlined residues being directly involved in ACE2 binding:

(SEQ ID NO: 100)
RVQPTESIVRFPNITNLCPFGEVFNATRFASVYAWNRKRISNCVADYS
VLYNSASFSTFKCYGVSPTKLNDLCFTNVYADSFVIRGDEVRQIAPGQ
TGKIADYNYKLPDDFTGCVIAWNSNNLDSKVGGNYNYLYRLFRKSNLK
PFERDISTEIYQAGSTPCNGVEGFNCYFPLQSYGFQPTNGVGYQ.

An in vitro fluorescence polarization (FP) assay or similar technique is performed according to standard procedures to determine the affinity of each peptide to the recombinant ACE2 protein.

Ligands (i.e., peptides) with the strongest affinities to ACE2 receptor are selected and attached to nanoparticles (e.g., LNPs).

The ability of single ligand and dual-ligand nanoparticles to target ACE2 receptor is determined. For example, the targeting ability of nanoparticles containing the ligand with the highest affinity to ACE2 receptor is compared to nanoparticles containing two different ligands having the highest affinities to ACE2 receptor.

Multiple ligand targeting is also tested using nanoparticles with one ligand that targets ACE2 receptor (e.g., to facilitate ACE2 receptor-mediated endocytosis) and a second ligand that is a nuclear localization signal (NLS) (e.g., to facilitate proper intracellular delivery via nuclear targeting).

SEQUENCES

SEQ ID NO: 1 membrane protein, amino acid sequence
MADSNGTITVEELKKLLEQWNLVIGFLFLTWICLLQFAYANRNRFLYIIKLIFLWLLWPVTLACF
VLAAVYRINWITGGIAIAMACLVGLMWLSYFIASFRLFARTRSMWSFNPETNILLNVPLHGTILT
RPLLESELVIGAVILRGHLRIAGHHLGRCDIKDLPKEITVATSRTLSYYKLGASQRVAGD
SGFAAYSRYRIGNYKLNTDHSSSSDNIALLVQ
SEQ ID NO: 2 membrane protein, nucleic acid sequence
atggcagattccaacggtactattaccgttgaagagcttaaaaagctccttgaacaatggaacct
agtaataggtttcctattccttacatggatttgtcttctacaatttgcctatgccaacaggaa
taggtttttgtatataattaagttaattttcctctggctgttatggccagtaactttagcttgtt
ttgtgcttgctgctgtttacagaataaattggatcaccggtggaattgctatcgcaatggcttgt
cttgtaggcttgatgtggctcagctacttcattgcttctttcagactgtttgcgcgtacgcgttc
catgtggtcattcaatccagaaactaacattcttctcaacgtgccactccatggcactattctga
ccagaccgcttctagaaagtgaactcgtaatcggagctgtgatccttcgtggacatcttc
gtattgctggacaccatctaggacgctgtgacatcaaggacctgcctaaagaaatcactgttgct
acatcacgaacgctttcttattacaaattgggagcttcgcagcgtgtagcaggtgactcaggttt
tgctgcatacagtcgctacaggattggcaactataaattaaacacagaccattccagtagcagtg
acaatattgctttgcttgtacagtaa
SEQ ID NO: 3 envelope protein, amino acid sequence
MYSFVSEETGTLIVNSVLLFLAFVVFLLVTLAILTALRLCAYCCNIVNVSLVKPSFYVYSRVKNL
NSSRVPDLLV
SEQ ID NO: 4 envelope protein, nucleic acid sequence
atgtactcattcgtttcggaagagacaggtacgttaatagttaatagcgtacttctttttcttgc
tttcgtggtattcttgctagttacactagccatccttactgcgcttcgattgtgtgcgtactgct
gcaatattgttaacgtgagtcttgtaaaaccttctttttacgtttactctcgtgttaaaaatctg
aattcttctagagttcctgatcttctggtctaa
SEQ ID NO: 5 spike protein, amino acid sequence
MFVFLVLLPLVSSQCVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNFKNLREFVFK
NIDGYFKIYSKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQTSNFRVQPTES
IVRFPNITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYLYRL
FRKSNLKPFERDISTEIYQAGSTPCNGVEGFNCYFPLQSYGFQPTNGVGYQPYRVVVLSFELLHA
PATVCGPKKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLGAENSVAYSNNSIAIP
TNFTISVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYRFNGIG
VTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVT
QRNFYEPQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWPWYIWLGFIAGLIAIVMVTIMLC
CMTSCCSCLKGCCSCGSCCKFDEDDSEPVLKGVKLHYT
SEQ ID NO: 6 spike protein, nucleic acid sequence
atgtttgtttttcttgttttattgccactagtctctagtcagtgtgttaatcttacaaccagaac
tcaattaccccctgcatacactaattctttcacacgtggtgtttattaccctgacaaagtttt
cagatcctcagttttacattcaactcaggacttgttcttacctttcttttccaatgttacttggt
tccatgctatacatgtctctgggaccaatggtactaagaggtttgataaccctgtcctaccattt
aatgatggtgtttattttgcttccactgagaagtctaacataataagaggctggatttttggtac
tactttagattcgaagacccagtccctacttattgttaataacgctactaatgttgttattaaag
tctgtgaatttcaattttgtaatgatccatttttgggtgtttattaccacaaaaacaacaaaagt
tggatggaaagtgagttcagagtttattctagtgcgaataattgcacttttgaatatgtctctca
gccttttcttatggaccttgaaggaaaacagggtaatttcaaaaatcttagggaatttgtgttta
agaatattgatggttattttaaaatatattctaagcacacgcctattaatttagtgcgtgatctc
cctcagggtttttcggctttagaaccattggtagatttgccaataggtattaacatcactaggtt
tcaaactttacttgctttacatagaagttatttgactcctggtgattcttcttcaggttggacag
ctggtgctgcagcttattatgtgggttatcttcaacctaggacttttctattaaaatataatgaa
aatggaaccattacagatgctgtagactgtgcacttgaccctctctcagaaacaaagtgtacgtt
gaaatccttcactgtagaaaaaggaatctatcaaacttctaactttagagtccaaccaacagaat
ctattgttagatttcctaatattacaaacttgtgcccttttggtgaagtttttaacgccaccaga
tttgcatctgtttatgcttggaacaggaagagaatcagcaactgtgttgctgattattctgtcct
atataattccgcatc
SEQ ID NO: 7 internal fusion peptide, amino acid sequence
SFIEDLLFNKVTLADAGF
SEQ ID NO: 8 internal fusion peptide, nucleic acid sequence
tcatttattgaagatctacttttcaacaaagtgacacttgcagatgctggcttc
SEQ ID NO: 9 receptor-binding domain, amino acid sequence
PNITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRVVVLSFELLHAPATV
CGPKKSTNLVKNKCVNFNFNGLTGTGVLTESNKKFLPFQQFGRDIADTTDAVRDPQTLE
SEQ ID NO: 10 receptor-binding domain, nucleic acid sequence
cctaatattacaaacttgtgcccttttggtgaagtttttaacgccaccagatttgcatctgttta
tgcttggaacaggaagagaatcagcaactgtgttgctgattattctgtcctatataattccgcat
cattttccacttttaagtgttatggagtgtctcctactaaattaaatgatctctgctttactaat
gtctatgcagattcatttgtaattagaggtgatgaagtcagacaaatcgctccagggcaaactgg
aaagattgctgattataattataaattaccagatgattttacaggctgcgttatagcttggaatt
ctaacaatcttgattctaaggttggtggtaattataattacctgtatagattgtttaggaagtct
aatctcaaaccttttgagagagatatttcaactgaaatctatcaggccggtagcacaccttgtaa
tggtgttgaaggttttaattgttactttcctttacaatcatatggtttccaacccactaatggtg
ttggttaccaaccatacagagtagtagtactttcttttgaacttctacatgcaccagcaactgtt
tgtggacctaaaaagtctactaatttggttaaaaacaaatgtgtcaatttcaacttcaatggttt
aacaggcacaggtgttcttactgagtctaacaaaaagtttctgcctttccaacaatttggcagag
acattgctgacactactgatgctgtccgtgatccacagacacttgag
SEQ ID NO: 11 immunogenic sequence, amino acid sequence
PNITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRVVVLSFELLHAP
SEQ ID NO: 12 conserved amino acid sequence
SFIEDL
SEQ ID NO: 13 conserved amino acid sequence
GVYYP
SEQ ID NO: 14 conserved amino acid sequence
FLPF
SEQ ID NO: 15 conserved amino acid sequence
VLPF
SEQ ID NO: 16 conserved amino acid sequence
SLLI
SEQ ID NO: 17 conserved amino acid sequence
LPIGI
SEQ ID NO: 18 conserved amino acid sequence
AAYYV
SEQ ID NO: 19 conserved amino acid sequence
TFLL
SEQ ID NO: 20 conserved amino acid sequence
AVDC
SEQ ID NO: 21 conserved amino acid sequence
IVRFP
SEQ ID NO: 22 conserved amino acid sequence
ISNC
SEQ ID NO: 23 conserved amino acid sequence
LCFT
SEQ ID NO: 24 conserved amino acid sequence
YNYKL
SEQ ID NO: 25 conserved amino acid sequence
IAWN
SEQ ID NO: 26 conserved amino acid sequence
VVVLSF
SEQ ID NO: 27 conserved amino acid sequence
CVNF
SEQ ID NO: 28 conserved amino acid sequence
GLTG
SEQ ID NO: 29 conserved amino acid sequence
VAVLY
SEQ ID NO: 30 conserved amino acid sequence
GCLI
SEQ ID NO: 31 conserved amino acid sequence
GIGA
SEQ ID NO: 32 conserved amino acid sequence
FTIS
SEQ ID NO: 33 conserved amino acid sequence
SVDC
SEQ ID NO: 34 conserved amino acid sequence
YGSFC
SEQ ID NO: 35 conserved amino acid sequence
FNFS
SEQ ID NO: 36 conserved amino acid sequence
RDLICAQ
SEQ ID NO: 37 conserved amino acid sequence
VLPPLL
SEQ ID NO: 38 conserved amino acid sequence
IPFA
SEQ ID NO: 39 conserved amino acid sequence
YRFN
SEQ ID NO: 40 conserved amino acid sequence
KLQDVVN
SEQ ID NO: 41 conserved amino acid sequence
GAISS
SEQ ID NO: 42 conserved amino acid sequence
EVQIDRLI
SEQ ID NO: 43 conserved amino acid sequence
YVTQQL
SEQ ID NO: 44 conserved amino acid sequence
HLMSF
SEQ ID NO: 45 conserved amino acid sequence
GVVHLF
SEQ ID NO: 46 conserved amino acid sequence
WFVT
SEQ ID NO: 47 conserved amino acid sequence
INAS
SEQ ID NO: 48 conserved amino acid sequence
LLQF
SEQ ID NO: 49 conserved amino acid sequence
LWLLWP
SEQ ID NO: 50 conserved amino acid sequence
LMWL
SEQ ID NO: 51 conserved amino acid sequence
SFRLF
SEQ ID NO: 52 conserved amino acid sequence
FNPETN
SEQ ID NO: 53 conserved amino acid sequence
ITVA
SEQ ID NO: 54 conserved amino acid sequence
LRLC
SEQ ID NO: 55 recombinant spike protein, amino acid sequence
PNITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRVVVLSFELLHAPGGG
GGGSFIEDLLFNKVTLADAGFGGGGGGWPWYIWLGFIAGLIAIVMVTIML
SEQ ID NO: 56 recombinant spike protein, nucleic acid sequence
ccaaacattaccaacctgtgccccttcggcgaggtgttcaacgccacacggttcgccagcgtgta
cgcctggaacagaaagcggatcagcaactgcgtggccgactacagtgtcctgtataactccgcca
gcttttctacattcaagtgctacggcgtctcccctaccaagctgaacgacctgtgcttcaccaat
gtgtacgccgattctttcgtgatcagaggcgacgaggtgcggcagatcgcccctggccagaccgg
aaagatcgctgattacaactacaagctgcctgatgacttcaccggctgcgtgatcgcctggaact
ccaacaacctggacagcaaggtggggggcaactacaactacctgtacagactgttcagaaagagc
aatctgaagcctttcgagagagatatcagcacagagatctaccaggccggcagcaccccttgtaa
tggcgttgagggcttcaattgctactttccactgcagagctatggctttcagcctacaaacggcg
tgggctaccaaccttacagagtggtggtgctgtctttcgagctgctgcacgcccctggcggagga
ggaggcggatctttcatcgaggacctgctgttcaacaaggtgaccctggccgacgccggttttgg
cggtggcggcggcggctggccttggtacatctggctgggcttcatcgccggactgatcgccatcg
tgatggtcaecatcatgctgtga
SEQ ID NO: 57 single open reading frame for coronavirus VLP, amino
acid sequence
MYSFVSEETGTLIVNSVLLFLAFVVFLLVTLAILTALRLCAYCCNIVNVSLVKPSFYVYSRVKNL
NSSRVPDLLVATNFSLLKQAGDVEENPGPMADSNGTITVEELKKLLEQWNLVIGFLFLTWICLLQ
FAYANRNRFLYIIKLIFLWLLWPVTLACFVLAAVYRINWITGGIAIAMACLVGLMWLSYFIASFR
LFARTRSMWSFNPETNILLNVPLHGTILTRPLLESELVIGAVILRGHLRIAGHHLGRCDIKDLPK
EITVATSRTLSYYKLGASQRVAGDSGFAAYSRYRIGNYKLNTDHSSSSDNIALLVQATNFSLLKQ
AGDVEENPGPPNITNLCPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFQPTNGVGYQPYRVVVLS
FELLHAPGGGGGGSFIEDLLFNKVTLADAGFGGGGGGWPWYIWLGFIAGLIAIVMVTIML
SEQ ID NO: 58 single open reading frame for coronavirus VLP, nucleic
acid sequence
atgtactctttcgtgtctgaggaaaccggcaccctgatcgtgaacagcgtgctgctgtttctggc
cttcgtggttttcctgctggtcaccctcgccatcctgaccgccctgcggctgtgcgcctactgct
gcaacatcgtgaacgtgtctctggtcaaacctagcttctacgtgtatagccgggtgaagaacctg
aattctagcagggtgcccgacctgctggtggccaccaacttcagcctgctgaaacaggctggcga
tgtggaagagaaccctggacctatggccgatagcaacggcaccattacagtggaggaactcaaaa
agctgctggaacagtggaatcttgtgatcggcttcctgttcctgacctggatctgcctgctgcag
ttcgcctacgccaaccgcaacagattcctgtacatcatcaaactgatcttcctgtggctgctgtg
gcccgtgaccctggcttgtttcgtgctggctgctgtttatagaatcaactggatcacaggcggca
tcgcaatcgccatggcctgtctggtgggcctgatgtggctgagctacttcatcgccagctttaga
ctgttcgctagaacaagaagcatgtggtcctttaaccccgagacaaacatcctcctgaatgtgcc
actgcatggcaccatcctgacaagacccctgctggaaagcgagctggtcatcggcgccgtgatcc
tgcggggccacctgagaatcgctggccaccacctgggcagatgtgacatcaaggacctgcccaag
gaaatcactgtggccacaagcagaaccctcagctactacaagctgggagcctctcagagagtggc
cggcgacagcggcttcgccgcctacagccggtaccggattggcaattacaaactgaacaccgacc
acagctccagcagcgacaacatcgctctgctagtgcaggccaccaatttcagcctgctgaagcaa
gctggagatgtggaagaaaaccccggccctccaaacattaccaacctgtgccccttcggcgaggt
gttcaacgccacacggttcgccagcgtgtacgcctggaacagaaagcggatcagcaactgcgtgg
ccgactacagtgtcctgtataactccgccagcttttctacattcaagtgctacggcgtctcccct
accaagctgaacgacctgtgcttcaccaatgtgtacgccgattctttcgtgatcagaggcgacga
ggtgcggcagatcgcccctggccagaccggaaagatcgctgattacaactacaagctgcctgatg
acttcaccggctgcgtgatcgcctggaactccaacaacctggacagcaaggtggggggcaactac
aactacctgtacagactgttcagaaagagcaatctgaagcctttcgagagagatatcagcacaga
gatctaccaggccggcagcaccccttgtaatggcgttgagggcttcaattgctactttccactgc
agagctatggctttcagcctacaaacggcgtgggctaccaaccttacagagtggtggtgctgtct
ttcgagctgctgcacgcccctggcggaggaggaggcggatctttcatcgaggacctgctgttcaa
caaggtgaccctggccgacgccggttttggcggtggcggcggcggctggccttggtacatctggc
tgggcttcatcgccggactgatcgccatcgtgatggtcaccatcatgctgtga
SEQ ID NO: 59 expression cassette for VLP, nucleic acid sequence
cgttacataacttacggtaaatggcccgcctggctgaccgcccaacgacccccgcccattgacgt
caataatgacgtatgttcccatagtaacgccaatagggactttccattgacgtcaatgggtggag
tatttacggtaaactgcccacttggcagtacatcaagtgtatcatatgccaagtacgccccctat
tgacgtcaatgacggtaaatggcccgcctggcattatgcccagtacatgaccttatgggactttc
ctacttggcagtacatctacgtattagtcatcgctattaccatggtgatgcggttttggcagtac
atcaatgggcgtggatagcggtttgactcacggggatttccaagtctccaccccattgacgtcaa
tgggagtttgttttggcaccaaaatcaacgggactttccaaaatgtcgtaacaactccgccccat
tgacgcaaatgggcggtaggcgtgtacggtgggaggtctatataagcagagctggtttagtgaac
cgtcagatccgctagcgccaccatgtactctttcgtgtctgaggaaaccggcaccctgatcgtga
acagcgtgctgctgtttctggccttcgtggttttcctgctggtcaccctcgccatcctgaccgcc
ctgcggctgtgcgcctactgctgcaacatcgtgaacgtgtctctggtcaaacctagcttctacgt
gtatagccgggtgaagaacctgaattctagcagggtgcccgacctgctggtggccaccaacttca
gcctgctgaaacaggctggcgatgtggaagagaaccctggacctatggccgatagcaacggcacc
attacagtggaggaactcaaaaagctgctggaacagtggaatcttgtgatcggcttcctgttcct
gacctggatctgcctgctgcagttcgcctacgccaaccgcaacagattcctgtacatcatcaaac
tgatcttcctgtggctgctgtggcccgtgaccctggcttgtttcgtgctggctgctgtttataga
atcaactggatcacaggcggcatcgcaatcgccatggcctgtctggtgggcctgatgtggctgag
ctacttcatcgccagctttagactgttcgctagaacaagaagcatgtggtcctttaaccccgaga
caaacatcctcctgaatgtgccactgcatggcaccatcctgacaagacccctgctggaaagcgag
ctggtcatcggcgccgtgatcctgcggggccacctgagaatcgctggccaccacctgggcagatg
tgacatcaaggacctgcccaaggaaatcactgtggccacaagcagaaccctcagctactacaagc
tgggagcctctcagagagtggccggcgacagcggcttcgccgcctacagccggtaccggattggc
aattacaaactgaacaccgaccacagctccagcagcgacaacatcgctctgctagtgcaggccac
caatttcagcctgctgaagcaagctggagatgtggaagaaaaccccggccctccaaacattacca
acctgtgccccttcggcgaggtgttcaacgccacacggttcgccagcgtgtacgcctggaacaga
aagcggatcagcaactgcgtggccgactacagtgtcctgtataactccgccagcttttctacatt
caagtgctacggcgtctcccctaccaagctgaacgacctgtgcttcaccaatgtgtacgccgatt
ctttcgtgatcagaggcgacgaggtgcggcagatcgcccctggccagaccggaaagatcgctgat
tacaactacaagctgcctgatgacttcaccggctgcgtgatcgcctggaactccaacaacctgga
cagcaaggtggggggcaactacaactacctgtacagactgttcagaaagagcaatctgaagcctt
tcgagagagatatcagcacagagatctaccaggccggcagcaccccttgtaatggcgttgagggc
ttcaattgctactttccactgcagagctatggctttcagcctacaaacggcgtgggctaccaacc
ttacagagtggtggtgctgtctttcgagctgctgcacgcccctggcggaggaggaggcggatctt
tcatcgaggacctgctgttcaacaaggtgaccctggccgacgccggttttggcggtggcggcggc
ggctggccttggtacatctggctgggcttcatcgccggactgatcgccatcgtgatggtcaccat
catgctgtgaacggccggctgatcataatcagccataccacatttgtagaggttttacttgcttt
aaaaaacctcccacacctccccctgaacctgaaacataaaatgaatgcaattgttgttgttaact
tgtttattgcagcttataatggttacaaataaagcaatagcatcacaaatttcacaaataaagca
tttttttcactgcattctagttgtggtttgtccaaactcatcaatgtatctta
SEQ ID NO: 60 expression cassette for VLP, nucleic acid sequence
cgttacataacttacggtaaatggcccgcctggctgaccgcccaacgacccccgcccattgacgt
caataatgacgtatgttcccatagtaacgccaatagggactttccattgacgtcaatgggtggag
tatttacggtaaactgcccacttggcagtacatcaagtgtatcatatgccaagtacgccccctat
tgacgtcaatgacggtaaatggcccgcctggcattatgcccagtacatgaccttatgggactttc
ctacttggcagtacatctacgtattagtcatcgctattaccatggtgatgcggttttggcagtac
atcaatgggcgtggatagcggtttgactcacggggatttccaagtctccaccccattgacgtcaa
tgggagtttgttttggcaccaaaatcaacgggactttccaaaatgtcgtaacaactccgccccat
tgacgcaaatgggcggtaggcgtgtacggtgggaggtctatataagcagagctggtttagtgaac
cgtcagatccgctagcgccaccatgtactctttcgtgtctgaggaaaccggcaccctgatcgtga
acagcgtgctgctgtttctggccttcgtggttttcctgctggtcaccctcgccatcctgaccgcc
ctgcggctgtgcgcctactgctgcaacatcgtgaacgtgtctctggtcaaacctagcttctacgt
gtatagccgggtgaagaacctgaattctagcagggtgcccgacctgctggtggccaccaacttca
gcctgctgaaacaggctggcgatgtggaagagaaccctggacctatggccgatagcaacggcacc
attacagtggaggaactcaaaaagctgctggaacagtggaatcttgtgatcggcttcctgttcct
gacctggatctgcctgctgcagttcgcctacgccaaccgcaacagattcctgtacatcatcaaac
tgatcttcctgtggctgctgtggcccgtgaccctggcttgtttcgtgctggctgctgtttataga
atcaactggatcacaggcggcatcgcaatcgccatggcctgtctggtgggcctgatgtggctgag
ctacttcatcgccagctttagactgttcgctagaacaagaagcatgtggtcctttaaccccgaga
caaacatcctcctgaatgtgccactgcatggcaccatcctgacaagacccctgctggaaagcgag
ctggtcatcggcgccgtgatcctgcggggccacctgagaatcgctggccaccacctgggcagatg
tgacatcaaggacctgcccaaggaaatcactgtggccacaagcagaaccctcagctactacaagc
tgggagcctctcagagagtggccggcgacagcggcttcgccgcctacagccggtaccggattggc
aattacaaactgaacaccgaccacagctccagcagcgacaacatcgctctgctagtgcaggccac
caatttcagcctgctgaagcaagctggagatgtggaagaaaaccccggccctccaaacattacca
acctgtgccccttcggcgaggtgttcaacgccacacggttcgccagcgtgtacgcctggaacaga
aagcggatcagcaactgcgtggccgactacagtgtcctgtataactccgccagcttttctacatt
caagtgctacggcgtctcccctaccaagctgaacgacctgtgcttcaccaatgtgtacgccgatt
ctttcgtgatcagaggcgacgaggtgcggcagatcgcccctggccagaccggaaagatcgctgat
tacaactacaagctgcctgatgacttcaccggctgcgtgatcgcctggaactccaacaacctgga
cagcaaggtggggggcaactacaactacctgtacagactgttcagaaagagcaatctgaagcctt
tcgagagagatatcagcacagagatctaccaggccggcagcaccccttgtaatggcgttgagggc
ttcaattgctactttccactgcagagctatggctttcagcctacaaacggcgtgggctaccaacc
ttacagagtggtggtgctgtctttcgagctgctgcacgcccctggcggaggaggaggcggatctt
tcatcgaggacctgctgttcaacaaggtgaccctggccgacgccggttttggcggtggcggcggc
ggctggccttggtacatctggctgggcttcatcgccggactgatcgccatcgtgatggtcaccat
catgctgtgactgtgccttctagttgccagccatctgttgtttgcccctcccccgtgccttcctt
gaccctggaaggtgccactcccactgtcctttcctaataaaatgaggaaattgcatcgcattgtc
tgagtaggtgtcattctattctggggggtggggtggggcaggacagcaagggggaggattgggaa
gacaatagcaggcatgctggggatgcggtgggctctatgg
SEQ ID NO: 61 expression cassette for VLP, nucleic acid sequence
cgttacataacttacggtaaatggcccgcctggctgaccgcccaacgacccccgcccattgacgt
caataatgacgtatgttcccatagtaacgccaatagggactttccattgacgtcaatgggtggag
tatttacggtaaactgcccacttggcagtacatcaagtgtatcatatgccaagtacgccccctat
tgacgtcaatgacggtaaatggcccgcctggcattatgcccagtacatgaccttatgggactttc
ctacttggcagtacatctacgtattagtcatcgctattaccatggtgatgcggttttggcagtac
atcaatgggcgtggatagcggtttgactcacggggatttccaagtctccaccccattgacgtcaa
tgggagtttgttttggcaccaaaatcaacgggactttccaaaatgtcgtaacaactccgccccat
tgacgcaaatgggcggtaggcgtgtacggtgggaggtctatataagcagagctggtttagtgaac
cgtcagatccgctagcgccaccatgtactctttcgtgtctgaggaaaccggcaccctgatcgtga
acagcgtgctgctgtttctggccttcgtggttttcctgctggtcaccctcgccatcctgaccgcc
ctgcggctgtgcgcctactgctgcaacatcgtgaacgtgtctctggtcaaacctagcttctacgt
gtatagccgggtgaagaacctgaattctagcagggtgcccgacctgctggtggccaccaacttca
gcctgctgaaacaggctggcgatgtggaagagaaccctggacctgccgatagcaacggcaccatt
acagtggaggaactcaaaaagctgctggaacagtggaatcttgtgatcggcttcctgttcctgac
ctggatctgcctgctgcagttcgcctacgccaaccgcaacagattcctgtacatcatcaaactga
tcttcctgtggctgctgtggcccgtgaccctggcttgtttcgtgctggctgctgtttatagaatc
aactggatcacaggcggcatcgcaatcgccatggcctgtctggtgggcctgatgtggctgagcta
cttcatcgccagctttagactgttcgctagaacaagaagcatgtggtcctttaaccccgagacaa
acatcctcctgaatgtgccactgcatggcaccatcctgacaagacccctgctggaaagcgagctg
gtcatcggcgccgtgatcctgcggggccacctgagaatcgctggccaccacctgggcagatgtga
catcaaggacctgcccaaggaaatcactgtggccacaagcagaaccctcagctactacaagctgg
gagcctctcagagagtggccggcgacagcggcttcgccgcctacagccggtaccggattggcaat
tacaaactgaacaccgaccacagctccagcagcgacaacatcgctctgctagtgcaggccaccaa
tttcagcctgctgaagcaagctggagatgtggaagaaaaccccggccctccaaacattaccaacc
tgtgccccttcggcgaggtgttcaacgccacacggttcgccagcgtgtacgcctggaacagaaag
cggatcagcaactgcgtggccgactacagtgtcctgtataactccgccagcttttctacattcaa
gtgctacggcgtctcccctaccaagctgaacgacctgtgcttcaccaatgtgtacgccgattctt
tcgtgatcagaggcgacgaggtgcggcagatcgcccctggccagaccggaaagatcgctgattac
aactacaagctgcctgatgacttcaccggctgcgtgatcgcctggaactccaacaacctggacag
caaggtggggggcaactacaactacctgtacagactgttcagaaagagcaatctgaagcctttcg
agagagatatcagcacagagatctaccaggccggcagcaccccttgtaatggcgttgagggcttc
aattgctactttccactgcagagctatggctttcagcctacaaacggcgtgggctaccaacctta
cagagtggtggtgctgtctttcgagctgctgcacgcccctggcggaggaggaggcggatctttca
tcgaggacctgctgttcaacaaggtgaccctggccgacgccggttttggcggtggcggcggcggc
tggccttggtacatctggctgggcttcatcgccggactgatcgccatcgtgatggtcaccateat
gctggagggcaggggaagtcttctaacatgcggggacgtggaggaaaatcccggcccagagagcg
acgagagcggcctgcccgccatggagatcgagtgccgcatcaccggcaccctgaacggcgtggag
ttegagetggtgggcggcggagagggcacccccgagcagggccgcatgaccaacaagatgaagag
caccaaaggcgccctgaccttcagcccctacctgctgagccacgtgatgggctacggcttctacc
acttcggcacctaccccagcggctacgagaaccccttcctgcacgccatcaacaacggcggctac
accaacaceegeategagaagtacgaggacggcggcgtgetgeaegtgagettcagetaccgcta
cgaggccggccgcgtgatcggcgacttcaaggtgatgggcaccggcttccccgaggacagcgtga
tcttcaccgacaagatcatccgcagcaacgccaccgtggagcacctgcaccccatgggcgataac
gatctggatggcagcttcacccgcaccttcagcctgcgcgacggcggctactacagctccgtggt
ggacagccacatgcacttcaagagcgccatccaccccagcatcctgcagaacgggggccccatgt
tcgccttccgccgcgtggaggaggatcacagcaacaccgagctgggcatcgtggagtaccagcac
gccttcaagaccccggatgcagatgccggtgaagaaagagtttaaacggccggctgatcataatc
agccataccacatttgtagaggttttacttgctttaaaaaacctcccacacctccccctgaacct
gaaacataaaatgaatgcaattgttgttgttaacttgtttattgcagcttataatggttacaaat
aaagcaatagcatcacaaatttcacaaataaagcatttttttcactgcattctagttgtggtttg
tccaaactcatcaatgtatctta
SEQ ID NO: 62 expression cassette for VLP, nucleic acid sequence
cgttacataacttacggtaaatggcccgcctggctgaccgcccaacgacccccgcccattgacgt
caataatgacgtatgttcccatagtaacgccaatagggactttccattgacgtcaatgggtggag
tatttacggtaaactgcccacttggcagtacatcaagtgtatcatatgccaagtacgccccctat
tgacgtcaatgacggtaaatggcccgcctggcattatgcccagtacatgaccttatgggactttc
ctacttggcagtacatctacgtattagtcatcgctattaccatggtgatgcggttttggcagtac
atcaatgggcgtggatagcggtttgactcacggggatttccaagtctccaccccattgacgtcaa
tgggagtttgttttggcaccaaaatcaacgggactttccaaaatgtcgtaacaactccgccccat
tgacgcaaatgggcggtaggcgtgtacggtgggaggtctatataagcagagctggtttagtgaac
cgtcagatccgctagcgccaccatgtactctttcgtgtctgaggaaaccggcaccctgatcgtga
acagcgtgctgctgtttctggccttcgtggttttcctgctggtcaccctcgccatcctgaccgcc
ctgcggctgtgcgcctactgctgcaacatcgtgaacgtgtctctggtcaaacctagcttctacgt
gtatagccgggtgaagaacctgaattctagcagggtgcccgacctgctggtggccaccaacttca
gcctgctgaaacaggctggcgatgtggaagagaaccctggacctgccgatagcaacggcaccatt
acagtggaggaactcaaaaagctgctggaacagtggaatcttgtgatcggcttcctgttcctgac
ctggatctgcctgctgcagttcgcctacgccaaccgcaacagattcctgtacatcatcaaactga
tcttcctgtggctgctgtggcccgtgaccctggcttgtttcgtgctggctgctgtttatagaatc
aactggatcacaggcggcatcgcaatcgccatggcctgtctggtgggcctgatgtggctgagcta
cttcatcgccagctttagactgttcgctagaacaagaagcatgtggtcctttaaccccgagacaa
acatcctcctgaatgtgccactgcatggcaccatcctgacaagacccctgctggaaagcgagctg
gtcatcggcgccgtgatcctgcggggccacctgagaatcgctggccaccacctgggcagatgtga
catcaaggacctgcccaaggaaatcactgtggccacaagcagaaccctcagctactacaagctgg
gagcctctcagagagtggccggcgacagcggcttcgccgcctacagccggtaccggattggcaat
tacaaactgaacaccgaccacagctccagcagcgacaacatcgctctgctagtgcaggagggcag
gggaagtcttctaacatgcggggacgtggaggaaaatcccggcccaagacccaagctggctagcc
tcgagtctagagggcccgtttaaacccgctgatcagcctcgaggtaccggatccgcggccgcgat
atctctagactgtgccttctagttgccagccatctgttgtttgcccctcccccgtgccttccttg
accctggaaggtgccactcccactgtcctttcctaataaaatgaggaaattgcatcgcattgtct
gagtaggtgtcattctattctggggggtggggtggggcaggacagcaagggggaggattgggaag
acaatagcaggcatgctggggatgcggtgggctctatgg
SEQ ID NO: 63 expression vector with expression cassette for VLP,
nucleic acid sequence
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgattctgtggat
aaccgtattaccgccatgcattagttattaatagtaatcaattacggggtcattagttcatagcc
catatatggagttccgcgttacataacttacggtaaatggcccgcctggctgaccgcccaacgac
ccccgcccattgacgtcaataatgacgtatgttcccatagtaacgccaatagggactttccattg
acgtcaatgggtggagtatttacggtaaactgcccacttggcagtacatcaagtgtatcatatgc
caagtacgccccctattgacgtcaatgacggtaaatggcccgcctggcattatgcccagtacatg
accttatgggactttcctacttggcagtacatctacgtattagtcatcgctattaccatggtgat
gcggttttggcagtacatcaatgggcgtggatagcggtttgactcacggggatttccaagtctcc
accccattgacgtcaatgggagtttgttttggcaccaaaatcaacgggactttccaaaatgtcgt
aacaactccgccccattgacgcaaatgggcggtaggcgtgtacggtgggaggtctatataagcag
agctggtttagtgaaccgtcagatccgctagcgccaccatgtactctttcgtgtctgaggaaacc
ggcaccctgatcgtgaacagcgtgctgctgtttctggccttcgtggttttcctgctggtcaccct
cgccatcctgaccgccctgcggctgtgcgcctactgctgcaacatcgtgaacgtgtctctggtca
aacctagcttctacgtgtatagccgggtgaagaacctgaattctagcagggtgcccgacctgctg
gtggccaccaacttcagcctgctgaaacaggctggcgatgtggaagagaaccctggacctatggc
cgatagcaacggcaccattacagtggaggaactcaaaaagctgctggaacagtggaatcttgtga
tcggcttcctgttcctgacctggatctgcctgctgcagttcgcctacgccaaccgcaacagattc
ctgtacatcatcaaactgatcttcctgtggctgctgtggcccgtgaccctggcttgtttcgtgct
ggctgctgtttatagaatcaactggatcacaggcggcatcgcaatcgccatggcctgtctggtgg
gcctgatgtggctgagctacttcatcgccagctttagactgttcgctagaacaagaagcatgtgg
tcctttaaccccgagacaaacatcctcctgaatgtgccactgcatggcaccatcctgacaagacc
cctgctggaaagcgagctggtcatcggcgccgtgatcctgcggggccacctgagaatcgctggcc
accacctgggcagatgtgacatcaaggacctgcccaaggaaatcactgtggccacaagcagaacc
ctcagctactacaagctgggagcctctcagagagtggccggcgacagcggcttcgccgcctacag
ccggtaccggattggcaattacaaactgaacaccgaccacagctccagcagcgacaacatcgctc
tgctagtgcaggccaccaatttcagectgctgaagcaagctggagatgtggaagaaaaccccggc
cctccaaacattaccaacctgtgccccttcggcgaggtgttcaacgccacacggttcgccagcgt
gtacgcctggaacagaaagcggatcagcaactgcgtggccgactacagtgtcctgtataactccg
ccagcttttctacattcaagtgctacggcgtctcccctaccaagctgaacgacctgtgcttcacc
aatgtgtacgccgattctttcgtgatcagaggcgacgaggtgcggcagatcgcccctggccagac
cggaaagatcgctgattacaactacaagctgcctgatgacttcaccggctgcgtgatcgcctgga
actccaacaacctggacagcaaggtggggggcaactacaactacctgtacagactgttcagaaag
agcaatctgaagcctttcgagagagatatcagcacagagatctaccaggccggcagcaccccttg
taatggcgttgagggcttcaattgctactttccactgcagagctatggctttcagcctacaaacg
gcgtgggctaccaaccttacagagtggtggtgctgtctttcgagctgctgcacgcccctggcgga
ggaggaggcggatctttcatcgaggacctgctgttcaacaaggtgaccctggccgacgccggttt
tggcggtggcggcggcggctggccttggtacatctggctgggcttcatcgccggactgatcgcca
tcgtgatggtcaccatcatgctgtgactgtgccttctagttgccagccatctgttgtttgcccct
cccccgtgccttccttgaccctggaaggtgccactcccactgtcctttcctaataaaatgaggaa
attgcatcgcattgtctgagtaggtgtcattctattctggggggtggggtggggcaggacagcaa
gggggaggattgggaagacaatagcaggcatgctggggatgcggtgggctctatggaagcttacg
cgtggccgctcgagacgcaattcggcttggtgtggaaagtccccaggctccccagcaggcagaag
tatgcaaagcatgcatctcaattagtcagcaaccaggtgtggaaagtccccaggctccccagcag
gcagaagtatgcaaagcatgcatctcaattagtcagcaaccaaattaaagtaacccataacttcg
tatagcatacattatacgaagttatgaagttcctattctctagaaagtataggaacttctagtca
cctatttcagcatactacgcgcgtagtatgctgaaataggtttatcagcacacaatagtccatta
tacgcgcgtataatggcaattgtgtgctgattgggttactttaatttggatccgtcgaccgatgc
ccttgagagccttcaacccagtcagetccttccggtgggcgcggggcatgactatcgtcgccgca
cttatgactgtcttctttatcatgcaactcgtaggacaggtgccggcagcgctcttccgcttcct
cgctcactgactcgctgcgctcggtcgttcggctgcggcgagcggtatcagctcactcaaaggcg
gtaatacggttatccacagaatcaggggataacgcaggaaagaacatgtgagcaaaaggccagca
aaaggccaggaaccgtaaaaaggccgcgttgctggcgtttttccataggctccgcccccctgacg
agcatcacaaaaatcgacgctcaagtcagaggtggcgaaacccgacaggactataaagataccag
gcgtttccccctggaagctccctcgtgcgctctcctgttccgaccctgccgcttaccggatacct
gtccgcctttctcccttcgggaagcgtggcgctttctcatagctcacgctgtaggtatctcagtt
cggtgtaggtcgttcgctccaagctgggctgtgtgcacgaaccccccgttcagcccgaccgctgc
gccttatccggtaactatcgtcttgagtccaacccggtaagacacgacttatcgccactggcagc
agccactggtaacaggattagcagagcgaggtatgtaggcggtgctacagagttcttgaagtggt
ggcctaactacggctacactagaagaacagtatttggtatctgcgctctgctgaagccagttacc
ttcggaaaaagagttggtagctcttgatccggcaaacaaaccaccgctggtagcggtggtttttt
tgtttgcaagcagcagattacgcgcagaaaaaaaggatctcaagaagatcctttgatcttttcta
cggggtctgacgctcagtggaacgaaaactcacgttaagggattttggtcatgagattatcaaaa
aggatcttcacctagatccttttaaattaaaaatgaagttttaaatcaatctaaagtatatatga
gtaaacttggtctgacagttaccaatgcttaatcagtgaggcacctatctcagcgatctgtctat
ttcgttcatccatagttgcctgactccccgtcgtgtagataactacgatacgggagggcttacca
tctggccccagtgctgcaatgataccgcgagacccacgctcaccggctccagatttatcagcaat
aaaccagccagccggaagggccgagcgcagaagtggtcctgcaactttatccgcctccatccagt
ctattaattgttgccgggaagctagagtaagtagttcgccagttaatagtttgcgcaacgttgtt
gccattgctacaggcatcgtggtgtcacgctcgtcgtttggtatggcttcattcagctccggttc
ccaacgatcaaggcgagttacatgatcccccatgttgtgcaaaaaagcggttagctccttcggtc
ctccgatcgttgtcagaagtaagttggccgcagtgttatcactcatggttatggcagcactgcat
aattctcttactgtcatgccatccgtaagatgcttttctgtgactggtgagtactcaaccaagtc
attctgagaatagtgtatgcggcgaccgagttgctcttgcccggcgtcaatacgggataataccg
cgccacatagcagaactttaaaagtgctcatcattggaaaacgttcttcggggcgaaaactctca
aggatcttaccgctgttgagatccagttcgatgtaacccactcgtgcacccaactgatcttcagc
atcttttactttcaccagcgtttctgggtgagcaaaaacaggaaggcaaaatgccgcaaaaaagg
gaataagggcgacacggaaatgttgaatactcatactcttcctttttcaatattattgaagcatt
tatcagggttattgtctcatgagcggatacatatttgaatgtatttagaaaaataaacaaatagg
ggttccgcgcacatttccccgaaaagtgccacctgacgcgccctgtagcggcgcattaagcgcgg
cgggtgtggtggttacgcgcagcgtgaccgctacacttgccagcgccctagcgcccgctcctttc
gctttcttcccttcctttctcgccacgttcgccggctttccccgtcaagctctaaatcgggggct
ccctttagggttccgatttagtgctttacggcacctcgaccccaaaaaacttgattagggtgatg
gttcacgtagtgggccatcgccctgatagacggtttttcgccctttgacgttggagtccacgttc
tttaatagtggactcttgttccaaactggaacaacactcaaccctatctcggtctattcttttga
tttataagggattttgccgatttcggcctattggttaaaaaatgagctgatttaacaaaaattta
acgcgaattttaacaaaatattaacgcttacaatttgccattcgccattcaggctgcgcaactgt
tgggaagggcgatcggtgcgggcctcttcgctattacgccagcccaagctaccatgataagtaag
taatattaaggtacgtggaggttttacttgctttaaaaaacctcccacacctccccctgaacctg
aaacataaaatgaatgcaattgttgttgttaacttgtttattgcagcttataatggttacaaata
aagcaatagcatcacaaatttcacaaataaagcatttttttcactgcattctagttgtggtttgt
ccaaactcatcaatgtatcttatggtactgtaactgagctaacataa
SEQ ID NO: 64 Forward Primer, envelope protein, nucleic acid sequence
actgctgcaacatcgtgaac
SEQ ID NO: 65 Reverse Primer, envelope protein, nucleic acid sequence
tgctagaattcaggttcttcacc
SEQ ID NO: 66 Forward Primer, membrane protein, nucleic acid sequence
ttcctgtggctgctgtgg
SEQ ID NO: 67 Reverse Primer, membrane protein, nucleic acid sequence
atgaccagctcgctttccag
SEQ ID NO: 68 Forward Primer, receptor-binding domain, nucleic acid
sequence
atcagcacagagatctaccagg
SEQ ID NO: 69 Reverse Primer, receptor-binding domain, nucleic acid
sequence
agcaccaccactctgtaagg
SEQ ID NO: 70 ACE2 receptor peptide, amino acid sequence
STIEEQAKTFLDKFNHEAEDLFYQSSLASWNYNTNITEENVQNMNNAGDKWSAFLKEQSTLAQMY
SEQ ID NO: 71 BAP tag, amino acid sequence
GLNDIFEAQKIEWHE
SEQ ID NO: 72 ACE2 receptor peptide with C-terminal BAP tag, amino acid
sequence
STIEEQAKTFLDKFNHEAEDLFYQSSLASWNYNTNITEENVQNMNNAGDKWSAFLKEQSTLAQMY
GLNDIFEAQKIEWHE
SEQ ID NO: 73 ACE2 receptor peptide with C-terminal BAP tag, nucleic acid
sequence
tccactattgaagaacaggcaaagactttcttggacaaattcaaccacgaggccgaagacttgtt
ctatcaaagttcccttgcgagttggaattacaatacgaatatcaccgaagaaaacgttcagaata
tgaacaatgcaggcgacaaatggtccgcctttttgaaagaacaaagtaccctggcccagatgtac
ggtcttaatgacatctttgaagcgcaaaagatcgagtggcacgaa
SEQ ID NO: 74 ACE2 receptor peptide with N-terminal BAP tag, amino acid
sequence
GLNDIFEAQKIEWHESTIEEQAKTFLDKFNHEAEDLFYQSSLASWNYNTNITEENVQNMNNAGDK
WSAFLKEQSTLAQMY
SEQ ID NO: 75 ACE2 receptor peptide with N-terminal BAP tag, nucleic acid
sequence
ggtcttaatgacatctttgaagcgcaaaagatcgagtggcacgaatccactattgaagaacaggc
aaagactttcttggacaaattcaaccacgaggccgaagacttgttctatcaaagttcccttgcga
gttggaattacaatacgaatatcaccgaagaaaacgttcagaatatgaacaatgcaggcgacaaa
tggtccgcctttttgaaagaacaaagtaccctggcccagatgtac
SEQ ID NO: 76 ACE2 binding peptide, amino acid sequence
QSYGFQPTN
SEQ ID NO: 77 ACE2 binding peptide, amino acid sequence
LQSYGFQPTN
SEQ ID NO: 78 ACE2 binding peptide, amino acid sequence
QSYGFQPTNGVGY
SEQ ID NO: 79 ACE2 binding peptide, amino acid sequence
QPTNGVGY
SEQ ID NO: 80 ACE2 binding peptide, amino acid sequence
FQPTNGVGY
SEQ ID NO: 81 ACE2 binding peptide, amino acid sequence
QPTN
SEQ ID NO: 82 ACE2 binding peptide, amino acid sequence
FQPTN
SEQ ID NO: 83 ACE2 binding peptide, amino acid sequence
FQPTNGV
SEQ ID NO: 84 ACE2 binding peptide, amino acid sequence
TNGVGY
SEQ ID NO: 85 ACE2 binding peptide, amino acid sequence
FNCYFPLQ
SEQ ID NO: 86 ACE2 binding peptide, amino acid sequence
GFNCYFPLQ
SEQ ID NO: 87 ACE2 binding peptide, amino acid sequence
EGFN
SEQ ID NO: 88 ACE2 binding peptide, amino acid sequence
VEGFNCY
SEQ ID NO: 89 ACE2 binding peptide, amino acid sequence
EGFNCYFPLQ
SEQ ID NO: 90 ACE2 binding peptide, amino acid sequence
YNYLY
SEQ ID NO: 91 ACE2 binding peptide, amino acid sequence
NYNYLYR
SEQ ID NO: 92 ACE2 binding peptide, amino acid sequence
SFIEDLLFNKVTLADAGF
SEQ ID NO: 93 ACE2 binding peptide, amino acid sequence
SFIEDLLFNKVTLADAGFMKQYGCGKKKK
SEQ ID NO: 94 ACE2 binding peptide, amino acid sequence
SFIEDLLF
SEQ ID NO: 95 ACE2 binding peptide, amino acid sequence
SFIEDLLFGCGKKKK
SEQ ID NO: 96 ACE2 binding peptide, amino acid sequence
SFIEDLLFNKVTLADAGFMKQY
SEQ ID NO: 97 ACE2 binding peptide, amino acid sequence
SFIEDAAAGCGKKKK
SEQ ID NO: 98 ACE2 binding peptide, amino acid sequence
SFIEDAAA
SEQ ID NO: 99 ACE2 binding peptide, amino acid sequence
TRYYYLNYNYTTGY
SEQ ID NO: 100 ACE2 binding control peptide, amino acid sequence
RVQPTESIVRFPNITNLCPFGEVFNATRFASVYAWNRKRISNCVADYSVLYNSASFSTFKCYGVS
PTKLNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQ
SEQ ID NO: 101 immunogenic sequence, nucleic acid sequence
cctaatattacaaacttgtgcccttttggtgaagtttttaacgccaccagatttgcatctgttta
tgcttggaacaggaagagaatcagcaactgtgttgctgattattctgtcctatataattccgcat
cattttccacttttaagtgttatggagtgtctcctactaaattaaatgatctctgctttactaat
gtctatgcagattcatttgtaattagaggtgatgaagtcagacaaatcgctccagggcaaactgg
aaagattgctgattataattataaattaccagatgattttacaggctgcgttatagcttggaatt
ctaacaatcttgattctaaggttggtggtaattataattacctgtatagattgtttaggaagtct
aatctcaaaccttttgagagagatatttcaactgaaatctatcaggccggtagcacaccttgtaa
tggtgttgaaggttttaattgttactttcctttacaatcatatggtttccaacccactaatggtg
ttggttaccaaccatacagagtagtagtactttcttttgaacttctacatgcacca
SEQ ID NO: 102 transmembrane domain, amino acid sequence
WPWYIWLGFIAGL
SEQ ID NO: 103 transmembrane domain, nucleic acid sequence
tggccatggtacatttggctaggttttatagctggcttga
SEQ ID NO: 104 bacterial sequence-free vector, nucleic acid sequence
cgcgcgtagtatgctgaaataggtgactagaagttcctatactttctagagaataggaacttcat
aacttcgtataatgtatgctatacgaagttatgggttactttaatttggttgctgactaattgag
atgcatgctttgcatacttctgcctgctggggagcctggggactttccacacctggttgctgact
aattgagatgcatgctttgcatacttctgcctgctggggagcctggggactttccacacccctga
ttctgtggataaccgtattaccgccatgcattagttattaatagtaatcaattacggggtcatta
gttcatagcccatatatggagttccgcgttacataacttacggtaaatggcccgcctggctgacc
gcccaacgacccccgcccattgacgtcaataatgacgtatgttcccatagtaacgccaataggga
ctttccattgacgtcaatgggtggagtatttacggtaaactgcccacttggcagtacatcaagtg
tatcatatgccaagtacgccccctattgacgtcaatgacggtaaatggcccgcctggcattatgc
ccagtacatgaccttatgggactttcctacttggcagtacatctacgtattagtcatcgctatta
ccatggtgatgcggttttggcagtacatcaatgggcgtggatagcggtttgactcacggggattt
ccaagtctccaccccattgacgtcaatgggagtttgttttggcaccaaaatcaacgggactttcc
aaaatgtcgtaacaactccgccccattgacgcaaatgggcggtaggcgtgtacggtgggaggtct
atataagcagagctggtttagtgaaccgtcagatccgctagcgccaccatgtactctttcgtgtc
tgaggaaaccggcaccctgatcgtgaacagcgtgctgctgtttctggccttcgtggttttcctgc
tggtcaccctcgccatcctgaccgccctgcggctgtgcgcctactgctgcaacatcgtgaacgtg
tctctggtcaaacctagcttctacgtgtatagccgggtgaagaacctgaattctagcagggtgcc
cgacctgctggtggccaccaacttcagcctgctgaaacaggctggcgatgtggaagagaaccctg
gacctatggccgatagcaacggcaccattacagtggaggaactcaaaaagctgctggaacagtgg
aatcttgtgatcggcttcctgttcctgacctggatctgcctgctgcagttcgcctacgccaaccg
caacagattcctgtacatcatcaaactgatcttcctgtggctgctgtggcccgtgaccctggctt
gtttcgtgctggctgctgtttatagaatcaactggatcacaggcggcatcgcaatcgccatggcc
tgtctggtgggcctgatgtggctgagctacttcatcgccagctttagactgttcgctagaacaag
aagcatgtggtcctttaaccccgagacaaacatcctcctgaatgtgccactgcatggcaccatcc
tgacaagacccctgctggaaagcgagctggtcatcggcgccgtgatcctgcggggccacctgaga
atcgctggccaccacctgggcagatgtgacatcaaggacctgcccaaggaaatcactgtggccac
aagcagaaccctcagctactacaagctgggagcctctcagagagtggccggcgacagcggcttcg
ccgcctacagccggtaccggattggcaattacaaactgaacaccgaccacagctccagcagcgac
aacatcgctctgctagtgcaggccaccaatttcagcctgctgaagcaagctggagatgtggaaga
aaaccccggccctccaaacattaccaacctgtgccccttcggcgaggtgttcaacgccacacggt
tcgccagcgtgtacgcctggaacagaaagcggatcagcaactgcgtggccgactacagtgtcctg
tataactccgccagcttttctacattcaagtgctacggcgtctcccctaccaagctgaacgacct
gtgcttcaccaatgtgtacgccgattctttcgtgatcagaggcgacgaggtgcggcagatcgccc
ctggccagaccggaaagatcgctgattacaactacaagctgcctgatgacttcaccggctgcgtg
atcgcctggaactccaacaacctggacagcaaggtggggggcaactacaactacctgtacagact
gttcagaaagagcaatctgaagcctttcgagagagatatcagcacagagatctaccaggccggca
gcaccccttgtaatggcgttgagggcttcaattgctactttccactgcagagctatggctttcag
cctacaaacggcgtgggctaccaaccttacagagtggtggtgctgtctttcgagctgctgcacgc
ccctggcggaggaggaggcggatctttcatcgaggacctgctgttcaacaaggtgaccctggccg
acgccggttttggcggtggcggcggcggctggccttggtacatctggctgggcttcatcgccgga
ctgatcgccatcgtgatggtcaccatcatgctgtgactgtgccttctagttgccagccatctgtt
gtttgcccctcccccgtgccttccttgaccctggaaggtgccactcccactgtcctttcctaata
aaatgaggaaattgcatcgcattgtctgagtaggtgtcattctattctggggggtggggtggggc
aggacagcaagggggaggattgggaagacaatagcaggcatgctggggatgcggtgggctctatg
gaagcttacgcgtggccgctcgagacgcaattcggcttggtgtggaaagtccccaggctccccag
caggcagaagtatgcaaagcatgcatctcaattagtcagcaaccaggtgtggaaagtccccaggc
tccccagcaggcagaagtatgcaaagcatgcatctcaattagtcagcaaccaaattaaagtaacc
cataacttcgtatagcatacattatacgaagttatgaagttcctattctctagaaagtataggaa
cttctagtcacctatttcagcatactacgcgcg

The disclosure is not to be limited in scope by the specific embodiments described herein. Indeed, various modifications of the disclosure in addition to those described will become apparent to those skilled in the art from the foregoing description and accompanying figures. Such modifications are intended to fall within the scope of the appended claims.

Other embodiments are within the following claims.

1-146. (canceled)

147. An expression vector comprising:
an expression cassette that comprises a nucleic acid sequence encoding a recombinant protein comprising a conserved amino acid sequence from a virus fused to an immunogenic amino acid sequence,
a target sequence for a first recombinase flanking each side of the expression cassette, and
one or more additional target sequences for one or more additional recombinases integrated within non-binding regions of the target sequence for the first recombinase,
wherein protein expressed intracellularly from the expression cassette is capable of forming a virus-like particle (VLP).

148. The expression vector of claim 147, wherein:
(a) the immunogenic amino acid sequence is from the same virus as the conserved amino acid sequence,
(b) the conserved amino acid sequence is from a viral glycoprotein, optionally wherein the immunogenic amino acid sequence is from the same viral glycoprotein,
(c) the expression cassette further comprises a nucleic acid sequence encoding a viral envelope protein and/or a nucleic acid sequence encoding a viral matrix protein, optionally wherein the viral envelope protein and/or the viral matrix protein are from the same virus as the conserved amino acid sequence,
(d) the conserved amino acid sequence, the immunogenic amino acid sequence, the viral envelope protein, and/or the viral matrix protein is a consensus sequence,
(e) the recombinant protein is capable of stimulating an immune response against the virus comprising neutralizing antibodies, optionally wherein the immune response is cross-reactive to a related virus or strain,
(f) the recombinant protein is capable of stimulating a Th1 cell-mediated immune response against the virus, optionally wherein the immune response is cross-reactive to a related virus or strain,
(g) the recombinant protein excludes amino acid sequences from the virus that stimulate an immune response comprising non-neutralizing antibodies and/or that stimulate a Th2 cell-mediated immune response,
(h) the expression cassette comprises a single open reading frame comprising a nucleic acid sequence encoding a self-cleaving peptide between each nucleic acid sequence encoding a protein,
(i) the virus is a coronavirus, an influenza virus, a human immunodeficiency virus, a human papillomavirus, a hepatitis virus, or an oncolytic virus, or
(j) a combination thereof.

149. The expression vector of claim 147, wherein the virus is a coronavirus, optionally wherein the coronavirus is COVID-19.

150. The expression vector of claim 149, wherein the expression cassette comprises nucleic acid sequences encoding a coronavirus Membrane (M) protein, a coronavirus Envelope (E) protein, and a recombinant protein comprising a conserved amino acid sequence and an immunogenic amino acid sequence from a coronavirus Spike (S) protein.

151. The expression vector of claim 150, wherein:
(a) the conserved amino acid sequence is from the S protein S2′ cleavage site and internal fusion peptide (IFP),
(b) the conserved amino acid sequence comprises SEQ ID NO:12,
(c) the immunogenic amino acid sequence is from the S protein receptor-binding domain (RBD),
(d) the immunogenic amino acid sequence is at least about 90% identical to SEQ ID NO:11,
(e) the recombinant protein further comprises a transmembrane (TM) domain sequence from the S protein,
(f) the recombinant protein excludes amino acid sequences from the S protein that stimulate an immune response comprising non-neutralizing antibodies and/or that stimulate a Th2 cell-mediated immune response,
(g) the amino acid sequence of the recombinant protein is at least about 90% identical to SEQ ID NO:55,
(h) the expression cassette comprises a single open reading frame translated as an amino acid sequence at least about 90% identical to SEQ ID NO:57,
(i) the recombinant protein is capable of stimulating an immune response against COVID-19, optionally wherein the immune response is cross-reactive to other coronaviruses, further optionally wherein the immune response is cross-reactive to other severe acute respiratory syndrome coronaviruses and/or human betacoronaviruses,
(j) the recombinant protein is capable of stimulating a Th1 cell-mediated immune response against COVID-19, optionally wherein the immune response is cross-reactive to other coronaviruses, further optionally wherein the immune response is cross-reactive to other severe acute respiratory syndrome coronaviruses and/or human betacoronaviruses, or
(k) a combination thereof.

152. The expression vector of claim 147, wherein the target sequence for the first recombinase and the one or more additional target sequences for the one or more additional recombinases are selected from the group consisting of the PY54 pal site, the N15 telRL site, the loxP site, φK02 telRL site, the FRT site, the phiC31 attP site, and the λ attP site, optionally wherein the expression vector comprises each of the target sequences, further optionally wherein the expression vector comprises the Tel recombinase pal site and the telRL, loxP, and FRT recombinase target binding sequences integrated within the pal site.

153. The expression vector of claim 147, wherein the expression vector is for producing a bacterial sequence-free vector, optionally wherein the bacterial sequence-free vector has circular covalently closed ends or linear covalently closed ends.

154. A vector production system comprising recombinant cells designed to encode at least a first recombinase under the control of an inducible promoter, wherein the cells comprise the expression vector of claim 147.

155. A method of producing a bacterial sequence-free vector comprising incubating the vector production system of claim 154 under suitable conditions for expression of the first recombinase.

156. A bacterial sequence-free vector produced by the method of claim 155.

157. A bacterial sequence-free vector comprising an expression cassette that comprises a nucleic acid sequence encoding a recombinant protein comprising a conserved amino acid sequence from a virus fused to an immunogenic amino acid sequence, wherein protein expressed intracellularly from the expression cassette is capable of forming a VLP.

158. The bacterial sequence-free vector of claim 157, wherein:
(a) the immunogenic amino acid sequence is from the same virus as the conserved amino acid sequence,
(b) the conserved amino acid sequence is from a viral glycoprotein, optionally wherein the immunogenic amino acid sequence is from the same viral glycoprotein,
(c) the expression cassette further comprises a nucleic acid sequence encoding a viral envelope protein and/or a nucleic acid sequence encoding a viral matrix protein, optionally wherein the viral envelope protein and/or the viral matrix protein are from the same virus as the conserved amino acid sequence,
(d) the conserved amino acid sequence, the immunogenic amino acid sequence, the viral envelope protein, and/or the viral matrix protein is a consensus sequence,
(e) the recombinant protein is capable of stimulating an immune response against the virus comprising neutralizing antibodies, optionally wherein the immune response is cross-reactive to a related virus or strain,
(f) the recombinant protein is capable of stimulating a Th1 cell-mediated immune response against the virus, optionally wherein the immune response is cross-reactive to a related virus or strain,
(g) the recombinant protein excludes amino acid sequences from the virus that stimulate an immune response comprising non-neutralizing antibodies and/or that stimulate a Th2 cell-mediated immune response,
(h) the expression cassette comprises a single open reading frame comprising a nucleic acid sequence encoding a self-cleaving peptide between each nucleic acid sequence encoding a protein,
(i) the virus is a coronavirus, an influenza virus, a human immunodeficiency virus, a human papillomavirus, a hepatitis virus, or an oncolytic virus, or
(j) a combination thereof.

159. The bacterial sequence-free vector of claim 157, wherein the virus is a coronavirus, optionally wherein the coronavirus is COVID-19.

160. The bacterial sequence-free vector of claim 159, wherein the expression cassette comprises nucleic acid sequences encoding a coronavirus M protein, a coronavirus E protein, and a recombinant protein comprising a conserved amino acid sequence and an immunogenic amino acid sequence from a coronavirus S protein.

161. The bacterial sequence-free vector of claim 160, wherein:
(a) the conserved amino acid sequence is from the S protein ST cleavage site and IFP,
(b) the conserved amino acid sequence comprises SEQ ID NO:12,
(c) the immunogenic amino acid sequence is from the S protein RBD,
(d) the immunogenic amino acid sequence is at least about 90% identical to SEQ ID NO:11,
(e) the recombinant protein further comprises a TM domain sequence from the S protein,
(f) the recombinant protein excludes amino acid sequences from the S protein that stimulate an immune response comprising non-neutralizing antibodies and/or that stimulate a Th2 cell-mediated immune response,
(g) the amino acid sequence of the recombinant protein is at least about 90% identical to SEQ ID NO:55,
(h) the expression cassette comprises a single open reading frame translated as an amino acid sequence at least about 90% identical to SEQ ID NO:57,
(i) the recombinant protein is capable of stimulating an immune response against COVID-19, optionally wherein the immune response is cross-reactive to other coronaviruses, further optionally wherein the immune response is cross-reactive to other severe acute respiratory syndrome coronaviruses and/or human betacoronaviruses,
(j) the recombinant protein is capable of stimulating a Th1 cell-mediated immune response against COVID-19, optionally wherein the immune response is cross-reactive to other coronaviruses, further optionally wherein the immune response is cross-reactive to other severe acute respiratory syndrome coronaviruses and/or human betacoronaviruses, or
(k) a combination thereof.

162. The bacterial sequence-free vector of claim 157, further comprising at least one enhancer sequence flanking each side of the expression cassette, optionally wherein the at least one enhancer sequence is at least two enhancer sequences, further optionally wherein at least one enhancer sequence is a SV40 enhancer sequence.

163. The bacterial sequence-free vector of claim 157, comprising circular covalently closed ends or linear covalently closed ends.

164. A polynucleotide encoding an amino acid sequence at least about 90% identical to SEQ ID NO:57.

165. A recombinant cell comprising the expression vector of claim 147.

166. A method of producing a VLP, comprising culturing the recombinant cell of claim 165 under suitable conditions for production of the VLP from the expression vector.

167. The method of claim 166, further comprising isolating the VLP by affinity purification.

168. The method of claim 167, wherein the affinity purification comprises an angiotensin-converting enzyme 2 (ACE2) receptor peptide or an anti-S protein monoclonal antibody.

169. The method of claim 168, wherein:
(a) the ACE2 receptor peptide comprises an amino acid sequence that is at least about 90% identical to the amino acid sequence of SEQ ID NO:70,
(b) the ACE2 receptor peptide comprises a biotin acceptor peptide (BAP) tag at the C-terminus or N-terminus of the peptide, optionally wherein the BAP tag comprises an amino acid sequence at least about 90% identical to the amino acid sequence of SEQ ID NO:71,
(c) the ACE2 receptor peptide or anti-S protein monoclonal antibody is biotinylated and immobilized on a streptavidin-coated bead, or
(d) a combination thereof.

170. A VLP produced by the method of claim 166.

171. A VLP comprising a recombinant protein comprising a conserved amino acid sequence from a virus fused to an immunogenic amino acid sequence.

172. The VLP of claim 171, wherein:
(a) the immunogenic amino acid sequence is from the same virus as the conserved amino acid sequence,
(b) the conserved amino acid sequence is from a viral glycoprotein, optionally wherein the immunogenic amino acid sequence is from the same viral glycoprotein,
(c) the VLP further comprises a viral envelope protein and/or a viral matrix protein, optionally wherein the viral envelope protein and/or the viral matrix protein are from the same virus as the conserved amino acid sequence,
(d) the conserved amino acid sequence, the immunogenic amino acid sequence, the viral envelope protein, and/or the viral matrix protein is a consensus sequence,
(e) the recombinant protein is capable of stimulating an immune response against the virus comprising neutralizing antibodies, optionally wherein the immune response is cross-reactive to a related virus or strain,
(f) the recombinant protein is capable of stimulating a Th1 cell-mediated immune response against the virus, optionally wherein the immune response is cross-reactive to a related virus or strain,
(g) the recombinant protein excludes amino acid sequences from the virus that stimulate an immune response comprising non-neutralizing antibodies and/or that stimulate a Th2 cell-mediated immune response,
(h) the virus is a coronavirus, an influenza virus, a human immunodeficiency virus, a human papillomavirus, a hepatitis virus, or an oncolytic virus, or
(i) a combination thereof.

173. The VLP of claim 171, wherein the virus is a coronavirus, optionally wherein the coronavirus is COVID-19.

174. The VLP of claim 173, comprising a coronavirus Membrane (M) protein, a coronavirus Envelope (E) protein, and a recombinant protein comprising a conserved amino acid sequence and an immunogenic amino acid sequence from a coronavirus Spike (S) protein.

175. The VLP of claim 174, wherein:
(a) the conserved amino acid sequence is from the S protein S2′ cleavage site and internal fusion peptide (IFP),
(b) the conserved amino acid sequence comprises SEQ ID NO:12,
(c) the immunogenic amino acid sequence is from the S protein receptor-binding domain (RBD),
(d) the immunogenic amino acid sequence is at least about 90% identical to SEQ ID NO:11,
(e) the recombinant protein further comprises a transmembrane (TM) domain sequence from the S protein,
(f) the recombinant protein excludes amino acid sequences from the S protein that stimulate an immune response comprising non-neutralizing antibodies and/or that stimulate a Th2 cell-mediated immune response,
(g) the amino acid sequence of the recombinant protein is at least about 90% identical to SEQ ID NO:55,
(h) the amino acid sequence of the recombinant protein is at least about 90% identical to SEQ ID NO:55, the amino acid sequence of the M protein is at least about 90% identical to SEQ ID NO:1, and the amino acid sequence of the E protein is at least about 90% identical to SEQ ID NO:3,
(i) the recombinant protein is capable of stimulating an immune response against COVID-19, optionally wherein the immune response is cross-reactive to other coronaviruses, further optionally wherein the immune response is cross-reactive to other severe acute respiratory syndrome coronaviruses and/or human betacoronaviruses,
(j) the recombinant protein is capable of stimulating a Th1 cell-mediated immune response against COVID-19, optionally wherein the immune response is cross-reactive to other coronaviruses, further optionally wherein the immune response is cross-reactive to other severe acute respiratory syndrome coronaviruses and/or human betacoronaviruses, or
(k) a combination thereof.

176. A composition comprising the bacterial sequence-free vector of claim 157, optionally wherein the composition further comprises a delivery agent comprising a targeting ligand, further optionally wherein the targeting ligand comprises a S protein peptide comprising an amino acid sequence at least about 90% identical to any one of SEQ ID NOs:76-99.

177. A method of treating a viral infection in a subject, comprising administering to the subject bacterial sequence-free vector of claim 157, wherein intracellular expression of the bacterial sequence-free vector produces a VLP.
